(19) United States
(12) Patent Application Publication
(10) Pub. No.: US 2022/0192239 A1
(43) Pub. Date: Jun. 23, 2022

(54) EGG REPLACER AND COMPOSITIONS COMPRISING THE EGG REPLACER, AND METHODS FOR PRODUCING THE SAME

(71) Applicant: PERFECT DAY, INC., BERKELEY, CA (US)

(72) Inventors: TIMOTHY GEISTLINGER, OAKLAND, CA (US); TY B. WAGONER, EMERYVILLE, CA (US); RAVIRAJSINH P. JHALA, OAKLAND, CA (US); JONATHAN R. GLICKSBERG, EMERYVILLE, CA (US)

(21) Appl. No.: 17/605,475

(22) PCT Filed: Apr. 22, 2020
(86) PCT No.: PCT/US20/29391

§ 371 Date: Oct. 21, 2021

Related U.S. Application Data
(60) Provisional application No. 62/837,024, filed on Apr. 22, 2019.

(51) Int. Cl. A23L 15/00; A21D 13/066; A21D 2/26; A23J 3/10

(52) U.S. Cl. CPC A23L 15/35; A23J 3/10; A21D 2/263; A21D 13/066

(57) ABSTRACT

Provided is an egg replacer, and compositions comprising such egg replacer, and methods for producing the same.

### RELATED APPLICATIONS

This application claims priority to U.S. Provisional Patent Application Ser. No. 62/837,024, filed on Apr. 22, 2019, which is incorporated herein by reference, in its entirety.

### FIELD OF THE INVENTION

Provided is an egg replacer, and compositions comprising such egg replacer, and methods for producing the same.

### BACKGROUND OF THE INVENTION

Egg is a low caloric food source with a high content of easily digestible protein and an ideal balance of nutritionally indispensable amino acids. It comprises all 9 essential amino acids that cannot be synthesized by the human body, a protein digestibility-corrected amino acid score (PDCAAS) of 1 (compared to 0.92 for beef, 0.91 for soy, 0.67 for pea, or 0.45 for whole wheat), and has a biological value (BV) of 93.7 (compared to 76 for fish, 74.3 for beef, 72.8 for soybeans, 64 for rice or wheat, 60 for corn, and 58 for dry beans). In addition to their nutritional value, proteins comprised in egg impart functional attributes that permit the use of egg in preparing a wide variety of food products of pleasurable taste/flavor and texture, including emulsions, batters, doughs, baked/cooked goods, and egg-based food products. Eggs are useful in these applications because they contribute to processes such as gelling/thickening/coagulating, foaming/leavening, emulsifying, and water binding. Therefore, eggs are essential ingredients of many commonly consumed food products, imparting or contributing to volume, texture, emulsification, shape, taste/flavor, cooking stability, processing tolerance, and shelf life.

However, egg also comprises components that are considered unhealthy. Among such components are allergenic epitopes in proteins such as ovalbumin, ovomucoid, ovotransferrin, and lysozyme; allergenic epitopes in lipids such as LDL and livetins; disease-causing microbes such as Salmonella and E. coli bacteria and bird viruses (e.g., bird flu virus); and saturated fats and cholesterol. Moreover, avidin and phosvitin comprised in egg are anti-nutritive in that they bind dietary biotin and iron, respectively, thereby making biotin and iron biologically unavailable.

Many people also avoid eggs for personal or religious reasons, choosing, for example, to exclude animal products with a vegetarian or vegan diet. Such choices can be driven by culinary or health considerations, as well as by concerns over the use of antibiotics and hormones and cruel animal husbandry conditions in industrial chicken farming.

The disadvantageous aspects of egg have spurred development of egg replacers. Some of these efforts have centered on using natural egg white only, which is largely devoid of the above listed unhealthy components (albeit not of the also above mentioned problems associated with industrial chicken farming and food sourcing from animals), and replacing the functionalities and nutritional characteristics of the egg yolk with combinations of unsaturated oils, minerals, and vitamins. Other efforts have aimed to replace all egg components, for example, with plant-derived components. Such efforts have generally resulted in egg replacers that are devoid of just some of the above mentioned drawbacks of eggs, provide only some of the functionalities of egg protein, and/or have new drawbacks (e.g., lower nutritive content and/or problems with flavor, storage, and/or shelf-life).

Therefore, a need remains for an egg replacer that compared to egg provides similar nutritive content, similar functional attributes, and fewer or none of the undesired attributes.

### INCORPORATION BY REFERENCE

All publications, patents, patent applications, sequences, database entries, and other references mentioned herein are incorporated by reference in their entireties to the same extent as if each individual publication, patent, patent application, sequence, database entry, or other reference was specifically and individually indicated to be incorporated by reference. In case of conflict, the present specification, including definitions, will control.

### SUMMARY OF THE INVENTION

An egg replacer comprising a milk protein component consisting of a subset of whey milk proteins or of a subset of casein milk proteins or of a mixture of a subset of whey milk proteins and a subset of casein milk proteins, wherein the milk protein component imparts or materially contributes to at least one egg attribute of the egg replacer.

The egg replacer according to any of the above, wherein the milk protein component consists of one or more whey milk proteins.

The egg replacer according to any of the above, wherein the milk protein component consists of one or more casein milk proteins.

The egg replacer according to any of the above, wherein the milk protein component consists of a mixture of one or more whey milk proteins and one or more casein milk proteins.

The egg replacer according to any of the above, wherein the milk protein component consists of one or more milk proteins selected from the group consisting of β-lactoglobulin, α-lactalbumin, lactoferrin, transferrin, serum albumin, lactoperoxidase, GMP, β-casein, γ-casein, κ-casein, α-S1-casein, and α-S2-casein.

The egg replacer according to any of the above, wherein at least one of the one or more milk proteins comprised in the milk protein component is a recombinant milk protein.

The egg replacer according to any of the above, wherein the milk protein component comprises β-lactoglobulin.

The egg replacer according to any of the above, wherein the milk protein component consists of β-lactoglobulin.

The egg replacer according to any of the above, wherein the β-lactoglobulin is recombinant β-lactoglobulin that is at least 80% identical to bovine β-lactoglobulin.

The egg replacer according to any of the above, wherein the milk protein component comprises α-lactalbumin.

The egg replacer according to any of the above, wherein the milk protein component consists of α-lactalbumin.

The egg replacer according to any of the above, wherein the α-lactalbumin is recombinant α-lactalbumin that is at least 80% identical to bovine α-lactalbumin.

The egg replacer according to any of the above, wherein the milk protein component comprises β-casein.

The egg replacer according to any of the above, wherein the milk protein component consists of β-casein.

The egg replacer according to any of the above, wherein the β-casein is recombinant β-casein that is at least 80% identical to bovine β-casein.

The egg replacer according to any of the above, wherein the milk protein component comprises γ-casein.

The egg replacer according to any of the above, wherein the milk protein component consists of γ-casein.

The egg replacer according to any of the above, wherein the γ-casein is recombinant γ-casein that is at least 80% identical to bovine γ-casein.

The egg replacer according to any of the above, wherein the milk protein component comprises κ-casein.

The egg replacer according to any of the above, wherein the milk protein component consists of κ-casein.

The egg replacer according to any of the above, wherein the κ-casein is recombinant κ-casein that is at least 80% identical to bovine κ-casein.

The egg replacer according to any of the above, wherein the milk protein component comprises α-S1-casein.

The egg replacer according to any of the above, wherein the milk protein component consists of α-S1-casein.

The egg replacer according to any of the above, wherein the α-S1-casein is recombinant α-S1-casein that is at least 80% identical to bovine α-S1-casein.

The egg replacer according to any of the above, wherein the milk protein component comprises α-S2-casein.

The egg replacer according to any of the above, wherein the milk protein component consists of α-S2-casein.

The egg replacer according to any of the above, wherein the α-S2-casein is recombinant α-S2-casein that is at least 80% identical to bovine α-S2-casein.

The egg replacer according to any of the above, wherein the milk protein component comprises β-lactoglobulin and α-lactalbumin.

The egg replacer according to any of the above, wherein the milk protein component consists of β-lactoglobulin and α-lactalbumin.

The egg replacer according to any of the above, wherein one or both of the β-lactoglobulin and the α-lactalbumin are recombinant β-lactoglobulin and/or recombinant α-lactalbumin that are at least 80% identical to bovine β-lactoglobulin and/or bovine α-lactalbumin.

The egg replacer according to any of the above, wherein the milk protein component comprises β-lactoglobulin and β-casein.

The egg replacer according to any of the above, wherein the milk protein component consists of β-lactoglobulin and β-casein.

The egg replacer according to any of the above, wherein one or both of the β-lactoglobulin and the β-casein are recombinant β-lactoglobulin and/or recombinant β-casein that are at least 80% identical to bovine β-lactoglobulin and/or bovine β-casein.

The egg replacer according to any of the above, wherein the milk protein component comprises β-lactoglobulin and γ-casein.

The egg replacer according to any of the above, wherein the milk protein component consists of β-lactoglobulin and γ-casein.

The egg replacer according to any of the above, wherein one or both of the β-lactoglobulin and the γ-casein are recombinant β-lactoglobulin and/or recombinant γ-casein that are at least 80% identical to bovine β-lactoglobulin and/or bovine γ-casein.

The egg replacer according to any of the above, wherein the milk protein component comprises β-lactoglobulin and κ-casein.

The egg replacer according to any of the above, wherein the milk protein component consists of β-lactoglobulin and κ-casein.

The egg replacer according to any of the above, wherein one or both of the β-lactoglobulin and the κ-casein are recombinant β-lactoglobulin and/or recombinant κ-casein that are at least 80% identical to bovine β-lactoglobulin and/or bovine κ-casein.

The egg replacer according to any of the above, wherein the milk protein component comprises β-lactoglobulin and α-S1-casein.

The egg replacer according to any of the above, wherein the milk protein component consists of β-lactoglobulin and α-S1-casein.

The egg replacer according to any of the above, wherein one or both of the β-lactoglobulin and the α-S1-casein are recombinant β-lactoglobulin and/or recombinant α-S1-casein that are at least 80% identical to bovine β-lactoglobulin and/or bovine α-S1-casein.

The egg replacer according to any of the above, wherein the milk protein component comprises β-lactoglobulin and α-S2-casein.

The egg replacer according to any of the above, wherein the milk protein component consists of β-lactoglobulin and α-S1-casein.

The egg replacer according to any of the above, wherein one or both of the β-lactoglobulin and the α-S2-casein are recombinant β-lactoglobulin and/or recombinant α-S2-casein that are at least 80% identical to bovine β-lactoglobulin and/or bovine α-S2-casein.

The egg replacer according to any of the above, wherein the milk protein component comprises α-lactalbumin and β-casein.

The egg replacer according to any of the above, wherein the milk protein component consists of α-lactalbumin and β-casein.

The egg replacer according to any of the above, wherein one or both of the α-lactalbumin and the β-casein are recombinant α-lactalbumin and/or recombinant β-casein that are at least 80% identical to bovine α-lactalbumin and/or bovine β-casein.

The egg replacer according to any of the above, wherein the milk protein component comprises α-lactalbumin and γ-casein.

The egg replacer according to any of the above, wherein the milk protein component consists of α-lactalbumin and γ-casein.

The egg replacer according to any of the above, wherein one or both of the α-lactalbumin and the γ-casein are recombinant α-lactalbumin and/or recombinant γ-casein that are at least 80% identical to bovine α-lactalbumin and/or bovine γ-casein.

The egg replacer according to any of the above, wherein the milk protein component comprises α-lactalbumin and κ-casein.

The egg replacer according to any of the above, wherein the milk protein component consists of α-lactalbumin and κ-casein.

The egg replacer according to any of the above, wherein one or both of the α-lactalbumin and the κ-casein are recombinant α-lactalbumin and/or recombinant κ-casein that are at least 80% identical to bovine α-lactalbumin and/or bovine κ-casein.

The egg replacer according to any of the above, wherein the milk protein component comprises α-lactalbumin and α-S1-casein.

The egg replacer according to any of the above, wherein the milk protein component consists of α-lactalbumin and α-S1-casein.

The egg replacer according to any of the above, wherein one or both of the α-lactalbumin and the α-S1-casein are recombinant α-lactalbumin and/or recombinant α-S1-casein that are at least 80% identical to bovine α-lactalbumin and/or bovine α-S1-casein.

The egg replacer according to any of the above, wherein the milk protein component comprises α-lactalbumin and α-S2-casein.

The egg replacer according to any of the above, wherein the milk protein component consists of α-lactalbumin and α-S1-casein.

The egg replacer according to any of the above, wherein one or both of the α-lactalbumin and the α-S2-casein are recombinant α-lactalbumin and/or recombinant α-S2-casein that are at least 80% identical to bovine α-lactalbumin and/or bovine α-S2-casein.

The egg replacer according to any of the above, wherein at least one of the recombinant milk proteins comprised in the milk protein component is produced in a recombinant fungal or bacterial or algal or plant host cell.

The egg replacer according to any of the above, wherein the recombinant β-lactoglobulin is produced in a recombinant fungal or bacterial or algal or plant host cell.

The egg replacer according to any of the above, wherein the recombinant β-casein is produced in a recombinant fungal or bacterial or algal or plant host cell.

The egg replacer according to any of the above, wherein the recombinant α-lactalbumin is produced in a recombinant fungal or bacterial or algal or plant host cell.

The egg replacer according to any of the above, wherein the recombinant γ-casein is produced in a recombinant fungal or bacterial or algal or plant host cell.

The egg replacer according to any of the above, wherein the recombinant κ-casein is produced in a recombinant fungal or bacterial or algal or plant host cell.

The egg replacer according to any of the above, wherein the recombinant α-S1-casein is produced in a recombinant fungal or bacterial or algal or plant host cell.

The egg replacer according to any of the above, wherein the recombinant α-S2-casein is produced in a recombinant fungal or bacterial or algal or plant host cell.

The egg replacer according to any of the above, wherein the recombinant fungal host cell is a recombinant filamentous fungal host cell.

The egg replacer according to any of the above, wherein the recombinant fungal host cell is from a genus selected from the group consisting of: Aspergillus, Candida, Fusarium, Hansenula, Kluyveromyces, Pichia, Saccharomyces, Tetrahymena, Trichoderma, Yarrowia, and Zygosaccharomyces.

The egg replacer according to any of the above, wherein at least one of the recombinant milk proteins comprised in the milk protein component has a non-mammalian PTM.

The egg replacer according to any of the above, wherein the recombinant β-lactoglobulin has a non-mammalian PTM.

The egg replacer according to any of the above, wherein the recombinant α-lactalbumin has a non-mammalian PTM.

The egg replacer according to any of the above, wherein the recombinant β-casein has a non-mammalian PTM.

The egg replacer according to any of the above, wherein the recombinant γ-casein has a non-mammalian PTM.

The egg replacer according to any of the above, wherein the recombinant κ-casein has a non-mammalian PTM.

The egg replacer according to any of the above, wherein the recombinant α-S1-casein has a non-mammalian PTM.

The egg replacer according to any of the above, wherein the recombinant α-S2-casein has a non-mammalian PTM.

The egg replacer according to any of the above, wherein at least one of the recombinant milk proteins comprised in the milk protein component comprises a reactive site.

The egg replacer according to any of the above, wherein the recombinant β-lactoglobulin comprises a reactive site.

The egg replacer according to any of the above, wherein the recombinant α-lactalbumin comprises a reactive site.

The egg replacer according to any of the above, wherein the recombinant β-casein comprises a reactive site.

The egg replacer according to any of the above, wherein the recombinant γ-casein comprises a reactive site.

The egg replacer according to any of the above, wherein the recombinant κ-casein comprises a reactive site.

The egg replacer according to any of the above, wherein the recombinant α-S1-casein comprises a reactive site.

The egg replacer according to any of the above, wherein the recombinant α-S2-casein comprises a reactive site.

The egg replacer according to any of the above, wherein at least one of the recombinant milk proteins comprised in the milk protein component lacks a reactive site that is present in the corresponding native milk protein.

The egg replacer according to any of the above, wherein the recombinant β-lactoglobulin lacks a reactive site that is present in the corresponding native milk protein.

The egg replacer according to any of the above, wherein the recombinant α-lactalbumin lacks a reactive site that is present in the corresponding native milk protein.

The egg replacer according to any of the above, wherein the recombinant β-casein lacks a reactive site that is present in the corresponding native milk protein.

The egg replacer according to any of the above, wherein the recombinant γ-casein lacks a reactive site that is present in the corresponding native milk protein.

The egg replacer according to any of the above, wherein the recombinant κ-casein lacks a reactive site that is present in the corresponding native milk protein.

The egg replacer according to any of the above, wherein the recombinant α-S1-casein lacks a reactive site that is present in the corresponding native milk protein.

The egg replacer according to any of the above, wherein the recombinant α-S2-casein lacks a reactive site that is present in the corresponding native milk protein.

The egg replacer according to any of the above, wherein at least one of the recombinant milk proteins comprised in the milk protein component comprises a milk protein repeat.

The egg replacer according to any of the above, wherein the recombinant β-lactoglobulin comprises a milk protein repeat.

The egg replacer according to any of the above, wherein the recombinant α-lactalbumin comprises a milk protein repeat.

The egg replacer according to any of the above, wherein the recombinant β-casein comprises a milk protein repeat.

The egg replacer according to any of the above, wherein the recombinant γ-casein comprises a milk protein repeat.

The egg replacer according to any of the above, wherein the recombinant κ-casein comprises a milk protein repeat.

The egg replacer according to any of the above, wherein the recombinant α-S1-casein comprises a milk protein repeat.

The egg replacer according to any of the above, wherein the recombinant α-S2-casein comprises a milk protein repeat.

The egg replacer according to any of the above, wherein the egg replacer comprises between about 0.1% and about 100% by mass of the milk protein component.

The egg replacer according to any of the above, wherein the egg replacer comprises between about 1% and about 70% by mass of the milk protein component.

The egg replacer according to any of the above, wherein the egg replacer comprises between about 5% and about 40% by mass of the milk protein component.

The egg replacer according to any of the above, wherein the egg replacer comprises between about 10% and about 25% by mass of the milk protein component.

The egg replacer according to any of the above, wherein the egg replacer further comprises a carbohydrate.

The egg replacer according to any of the above, wherein the carbohydrate is obtained from a plant.

The egg replacer according to any of the above, wherein the carbohydrate is a monosaccharide, a disaccharide, a polysaccharide or a mixture thereof.

The egg replacer according to any of the above, wherein the carbohydrate is a starch, a gum, an edible fiber, a flour, or a mixture thereof.

The egg replacer according to any of the above, wherein the carbohydrate is a starch.

The egg replacer according to any of the above, wherein the starch is selected from the group consisting of tapioca starch, corn (maize) starch, potato starch, and mixtures thereof.

The egg replacer according to any of the above, wherein the egg replacer comprises between about 0.5% and about 40% by mass of the starch.

The egg replacer according to any of the above, wherein the egg replacer comprises between about 5% and about 30% by mass of the starch.

The egg replacer according to any of the above, wherein the egg replacer comprises between about 10% and about 20% by mass of the starch.

The egg replacer according to any of the above, wherein the carbohydrate is a gum.

The egg replacer according to any of the above, wherein the gum is selected from the group consisting of arrowroot flour, xanthan gum, tara gum, guar gum, agar gum, locust bean gum, and gum arabic, and mixtures thereof.

The egg replacer according to any of the above, wherein the egg replacer comprises between about 0.01%% and about 5% by mass of the gum.

The egg replacer according to any of the above, wherein the egg replacer comprises between about 0.5% and about 3% by mass of the gum.

The egg replacer according to any of the above, wherein the egg replacer comprises between about 1% and about 2.5% by mass of the gum.

The egg replacer according to any of the above, wherein the carbohydrate is an edible fiber.

The egg replacer according to any of the above, wherein the edible fiber is selected from the group consisting of psyllium husk, acacia fiber, citrus fiber, and cellulose, and mixtures thereof.

The egg replacer according to any of the above, wherein the egg replacer comprises between about 1% and about 30% by mass of the edible fiber.

The egg replacer according to any of the above, wherein the egg replacer comprises between about 5% and about 15% by mass of the edible fiber.

The egg replacer according to any of the above, wherein the carbohydrate is a flour.

The egg replacer according to any of the above, wherein the flour is selected from the group consisting of ground chia seeds, ground flaxseed, and sweet rice flour, and mixtures thereof.

The egg replacer according to any of the above, wherein the egg replacer comprises between about 1% and about 20% by mass of the flour.

The egg replacer according to any of the above, wherein the egg replacer comprises between about 5% and about 10% by mass of the flour.

The egg replacer according to any of the above, wherein the carbohydrate is a mixture of a starch and a gum.

The egg replacer according to any of the above, wherein the starch is selected from the group consisting of tapioca starch, corn (maize) starch, potato starch, arrowroot starch, tapioca syrup solids, and rice syrup solids, and mixtures thereof.

The egg replacer according to any of the above, wherein the gum is selected from the group consisting of arrowroot flour, xanthan gum, tara gum, guar gum, agar gum, locust bean gum, and gum arabic, and mixtures thereof.

The egg replacer according to any of the above, wherein the starch is selected from the group consisting of tapioca starch, corn (maize) starch, potato starch, arrowroot starch, tapioca syrup solids, and rice syrup solids, and mixtures thereof, and wherein the gum is selected from the group consisting of arrowroot flour, xanthan gum, tara gum, guar gum, agar gum, locust bean gum, and gum arabic, and mixtures thereof.

The egg replacer according to any of the above, wherein the egg replacer comprises between about 0.5% and about 40% by mass of the starch and between about 0.01% and about 5% by mass of the gum.

The egg replacer according to any of the above, wherein the egg replacer comprises between about 5% and about 30% by mass of the starch and between about 0.5% and about 3% by mass of the gum.

The egg replacer according to any of the above, wherein the egg replacer comprises between about 10% and about 20% by mass of the starch and between about 1% and about 2.5% by mass of the gum.

The egg replacer according to any of the above, wherein the carbohydrate is a mixture of a starch and an edible fiber.

The egg replacer according to any of the above, wherein the starch is selected from the group consisting of tapioca starch, corn (maize) starch, potato starch, arrowroot starch, tapioca syrup solids, and rice syrup solids, and mixtures thereof.

The egg replacer according to any of the above, wherein the edible fiber is selected from the group consisting of psyllium husk, acacia fiber, citrus fiber, and cellulose, and mixtures thereof.

The egg replacer according to any of the above, wherein the starch is selected from the group consisting of tapioca starch, corn (maize) starch, potato starch, arrowroot starch, tapioca syrup solids, and rice syrup solids, and mixtures thereof, and wherein the edible fiber is selected from the group consisting of psyllium husk, acacia fiber, citrus fiber, and cellulose, and mixtures thereof.

The egg replacer according to any of the above, wherein the egg replacer comprises between about 0.5% and about 40% by mass of the starch and between about 1% and about 30% by mass of the edible fiber.

The egg replacer according to any of the above, wherein the egg replacer comprises between about 5% and about 30% by mass of the starch and between about 5% and about 15% by mass of the edible fiber.

The egg replacer according to any of the above, wherein the carbohydrate is a mixture of a starch and a flour.

The egg replacer according to any of the above, wherein the starch is selected from the group consisting of tapioca starch, corn (maize) starch, potato starch, arrowroot starch, tapioca syrup solids, and rice syrup solids, and mixtures thereof.

The egg replacer according to any of the above, wherein the flour is selected from the group consisting of ground chia seeds, ground flaxseed, and sweet rice flour, and mixtures thereof.

The egg replacer according to any of the above, wherein the starch is selected from the group consisting of tapioca starch, corn (maize) starch, potato starch, arrowroot starch, tapioca syrup solids, and rice syrup solids, and mixtures thereof, and wherein the flour is selected from the group consisting of ground chia seeds, ground flaxseed, and sweet rice flour, and mixtures thereof.

The egg replacer according to any of the above, wherein the egg replacer comprises between about 0.5% and about 40% by mass of the starch and between about 1% and about 20% by mass of the flour.

The egg replacer according to any of the above, wherein the egg replacer comprises between about 5% and about 30% by mass of the starch and between about 5% and about 10% by mass of the flour.

The egg replacer according to any of the above, wherein the carbohydrate is a mixture of a starch and a gum and an edible fiber.

The egg replacer according to any of the above, wherein the starch is selected from the group consisting of tapioca starch, corn (maize) starch, potato starch, arrowroot starch, tapioca syrup solids, and rice syrup solids, and mixtures thereof.

The egg replacer according to any of the above, wherein the gum is selected from the group consisting of arrowroot flour, xanthan gum, tara gum, guar gum, agar gum, locust bean gum, and gum arabic, and mixtures thereof.

The egg replacer according to any of the above, wherein the edible fiber is selected from the group consisting of psyllium husk, acacia fiber, citrus fiber, and cellulose, and mixtures thereof.

The egg replacer according to any of the above, wherein the starch is selected from the group consisting of tapioca starch, corn (maize) starch, potato starch, arrowroot starch, tapioca syrup solids, and rice syrup solids, and mixtures thereof, and wherein the gum is selected from the group consisting of arrowroot flour, xanthan gum, tara gum, guar gum, agar gum, locust bean gum, and gum arabic, and mixtures thereof, and wherein the edible fiber is selected from the group consisting of psyllium husk, acacia fiber, citrus fiber, and cellulose, and mixtures thereof.

The egg replacer according to any of the above, wherein the egg replacer comprises between about 0.5% and about 40% by mass of the starch, and between about 0.5% and about 3% by mass of the gum, and between about 1% and about 30% by mass of the edible fiber.

The egg replacer according to any of the above, wherein the egg replacer comprises between about 5% and about 30% by mass of the starch, and between about 1% and about 2.5% by mass of the gum, and between about 5% and about 15% by mass of the edible fiber.

The egg replacer according to any of the above, wherein the carbohydrate is a mixture of a starch and a gum and a flour.

The egg replacer according to any of the above, wherein the starch is selected from the group consisting of tapioca starch, corn (maize) starch, potato starch, arrowroot starch, tapioca syrup solids, and rice syrup solids, and mixtures thereof.

The egg replacer according to any of the above, wherein the gum is selected from the group consisting of arrowroot flour, xanthan gum, tara gum, guar gum, agar gum, locust bean gum, and gum arabic, and mixtures thereof.

The egg replacer according to any of the above, wherein the flour is selected from the group consisting of ground chia seeds, ground flaxseed, and sweet rice flour, and mixtures thereof.

The egg replacer according to any of the above, wherein the starch is selected from the group consisting of tapioca starch, corn (maize) starch, potato starch, arrowroot starch, tapioca syrup solids, and rice syrup solids, and mixtures thereof, and wherein the gum is selected from the group consisting of arrowroot flour, xanthan gum, tara gum, guar gum, agar gum, locust bean gum, and gum arabic, and mixtures thereof, and wherein the flour is selected from the group consisting of ground chia seeds, ground flaxseed, and sweet rice flour, and mixtures thereof.

The egg replacer according to any of the above, wherein the egg replacer comprises between about 0.5% and about 40% by mass of the starch, and between about 0.5% and about 3% by mass of the gum, and between about 1% and about 20% by mass of the flour.

The egg replacer according to any of the above, wherein the egg replacer comprises between about 5% and about 30% by mass of the starch, and between about 1% and about 2.5% by mass of the gum, and between about 5% and about 10% by mass of the flour.

The egg replacer according to any of the above, wherein the carbohydrate is a mixture of a starch and an edible fiber and a flour.

The egg replacer according to any of the above, wherein the starch is selected from the group consisting of tapioca starch, corn (maize) starch, potato starch, arrowroot starch, tapioca syrup solids, and rice syrup solids, and mixtures thereof.

The egg replacer according to any of the above, wherein the edible fiber is selected from the group consisting of psyllium husk, acacia fiber, citrus fiber, and cellulose, and mixtures thereof.

The egg replacer according to any of the above, wherein the flour is selected from the group consisting of ground chia seeds, ground flaxseed, and sweet rice flour, and mixtures thereof.

The egg replacer according to any of the above, wherein the starch is selected from the group consisting of tapioca starch, corn (maize) starch, potato starch, arrowroot starch, tapioca syrup solids, and rice syrup solids, and mixtures thereof, and wherein the edible fiber is selected from the group consisting of psyllium husk, acacia fiber, citrus fiber, and cellulose, and mixtures thereof, and wherein the flour is selected from the group consisting of ground chia seeds, ground flaxseed, and sweet rice flour, and mixtures thereof.

The egg replacer according to any of the above, wherein the egg replacer comprises between about 0.5% and about 40% by mass of the starch, and between about 1% and about 30% by mass of the edible fiber, and between about 1% and about 20% by mass of the flour.

The egg replacer according to any of the above, wherein the egg replacer comprises between about 5% and about 30% by mass of the starch, and between about 5% and about 15% by mass of the edible fiber, and between about 5% and about 10% by mass of the flour.

The egg replacer according to any of the above, wherein the carbohydrate is a mixture of a gum and an edible fiber and a flour.

The egg replacer according to any of the above, wherein the gum is selected from the group consisting of arrowroot flour, xanthan gum, tara gum, guar gum, agar gum, locust bean gum, and gum arabic, and mixtures thereof.

The egg replacer according to any of the above, wherein the edible fiber is selected from the group consisting of psyllium husk, acacia fiber, citrus fiber, and cellulose, and mixtures thereof.

The egg replacer according to any of the above, wherein the flour is selected from the group consisting of ground chia seeds, ground flaxseed, and sweet rice flour, and mixtures thereof.

The egg replacer according to any of the above, wherein the gum is selected from the group consisting of tapioca starch, corn (maize) starch, potato starch, arrowroot starch, tapioca syrup solids, and rice syrup solids, and mixtures thereof, and wherein the edible fiber is selected from the group consisting of psyllium husk, acacia fiber, citrus fiber, and cellulose, and mixtures thereof, and wherein the flour is selected from the group consisting of ground chia seeds, ground flaxseed, and sweet rice flour, and mixtures thereof.

The egg replacer according to any of the above, wherein the egg replacer comprises between about 0.5% and about 3% by mass of the gum, and between about 1% and about 30% by mass of the edible fiber, and between about 1% and about 20% by mass of the flour.

The egg replacer according to any of the above, wherein the egg replacer comprises between about 1% and about 2.5% by mass of the gum, and between about 5% and about 15% by mass of the edible fiber, and between about 5% and about 10% by mass of the flour.

The egg replacer according to any of the above, wherein the carbohydrate is a mixture of a starch and a gum and an edible fiber and a flour.

The egg replacer according to any of the above, wherein the starch is selected from the group consisting of tapioca starch, corn (maize) starch, potato starch, arrowroot starch, tapioca syrup solids, and rice syrup solids, and mixtures thereof.

The egg replacer according to any of the above, wherein the gum is selected from the group consisting of arrowroot flour, xanthan gum, tara gum, guar gum, agar gum, locust bean gum, and gum arabic, and mixtures thereof.

The egg replacer according to any of the above, wherein the edible fiber is selected from the group consisting of psyllium husk, acacia fiber, citrus fiber, and cellulose, and mixtures thereof.

The egg replacer according to any of the above, wherein the flour is selected from the group consisting of ground chia seeds, ground flaxseed, and sweet rice flour, and mixtures thereof.

The egg replacer according to any of the above, wherein the starch is selected from the group consisting of tapioca starch, corn (maize) starch, potato starch, arrowroot starch, tapioca syrup solids, and rice syrup solids, and mixtures thereof, and wherein the gum is selected from the group consisting of arrowroot flour, xanthan gum, tara gum, guar gum, agar gum, locust bean gum, and gum arabic, and mixtures thereof, and wherein the edible fiber is selected from the group consisting of psyllium husk, acacia fiber, citrus fiber, and cellulose, and mixtures thereof, and wherein the flour is selected from the group consisting of ground chia seeds, ground flaxseed, and sweet rice flour, and mixtures thereof.

The egg replacer according to any of the above, wherein the egg replacer comprises between about 0.5% and about 40% by mass of the starch, and between about 0.5% and about 3% by mass of the gum, and between about 1% and about 30% by mass of the edible fiber, and between about 1% and about 20% by mass of the flour.

The egg replacer according to any of the above, wherein the egg replacer comprises between about 5% and about 30% by mass of the starch, and between about 1% and about 2.5% by mass of the gum, and between about 5% and about 15% by mass of the edible fiber, and between about 5% and about 10% by mass of the flour.

The egg replacer according to any of the above, wherein the egg attribute is selected from the group consisting of viscosity, density, nutrient content, foaming/leavening behavior, gelling/thickening/coagulating behavior, emulsifying behavior, water binding behavior, and use versatility.

The egg replacer according to any of the above, wherein the egg replacer upon heating comprise a milk protein polymer network.

The egg replacer according to any of the above, wherein the egg replacer is essentially free of gluten.

The egg replacer according to any of the above, wherein the egg replacer is essentially free of an egg protein.

The egg replacer according to any of the above, wherein the egg replacer is essentially free of one or more components obtained from an animal.

The egg replacer according to any of the above, wherein the egg replacer is essentially free of a saturated lipid.

The egg replacer according to any of the above, wherein the egg replacer is essentially free of cholesterol.

The egg replacer according to any of the above, wherein the egg replacer has an allergenicity that is lower than that of egg.

The egg replacer according to any of the above, wherein the egg replacer is a powder.

A composition comprising the egg replacer according to any of the above, wherein the egg replacer imparts on the composition or materially contributes to an attribute imparted by egg in a comparable composition.

The egg replacer according to any of the above, wherein the composition is a food product.

The composition according to any of the above, wherein the food product is a batter.

The composition according to any of the above, wherein the food product is a dough.

The composition according to any of the above, wherein the food product is a baked food product.

The composition according to any of the above, wherein the composition comprises between about 0.001% and 90% by mass of the egg replacer.

The composition according to any of the above, wherein the composition comprises between about 1% and 50% by mass of the egg replacer.

The composition according to any of the above, wherein the composition comprises between about 10% and 30% by mass of the egg replacer.

The composition according to any of the above, wherein the attribute is selected from the group consisting of color, browning, viscosity, density, rise/height, dome, spread, shape, hardness/firmness, adhesiveness, resilience/recoverable energy, structural integrity/cohesiveness, elasticity/springiness/rebound, chewiness/breakdown, taste/flavor, and eating quality.

The composition according to any of the above, wherein the baked food product is essentially free of egg.

The composition according to any of the above, wherein the baked food product is essentially free of gluten.

The composition according to any of the above, wherein the baked food product is essentially free of egg and gluten.

The composition according to any of the above, wherein the baked food product has a similar cake rise/height as a comparable baked food product comprising egg or gluten or both.

A method for producing the egg replacer according to any of the above, wherein the method comprises the step of obtaining one or more native and/or recombinant milk proteins, and optionally the step of combining the one or more native and/or recombinant milk proteins with one or more other ingredients.

The method according to any of the above further comprising post-processing of the one or more native and/or recombinant milk proteins comprising fragmenting, denaturating, removing reactive sites, modulating, cyclizing, biotinylating, and conjugation to other elements.

The method according to any of the above further comprising spray drying or concentrating the one or more native and/or recombinant milk proteins to obtain a powder.

### DETAILED DESCRIPTION OF THE INVENTION

The terminology and description used herein is for the purpose of describing particular embodiments only, and is not intended to limit the invention. Unless defined otherwise, all technical and scientific terms used herein have the same meaning as is commonly understood by one of ordinary skill in the art to which this disclosure pertains. Further, unless otherwise required by context, singular terms shall include the plural, and plural terms shall include the singular.

### Definitions

The terms “a” and “an” and “the” and similar references as used herein refer to both the singular and the plural (e.g., meaning “at least one” or “one or more”), unless otherwise indicated herein or clearly contradicted by context. For example, the term “a compound” or “at least one compound” or “one or more compounds” may include a plurality of compounds, including mixtures thereof.

The term “about” as used herein in conjunction with a stated numerical value or range of numerical values is meant to encompass variations of the stated numerical value or range of numerical values (i.e., denoting somewhat more or somewhat less than the stated numerical value or range of numerical values, to within a range of ±20%, ±10%, ±5%, ±1%, ±0.5%, ±0.1%, or ±one standard deviation of the stated value or range of numerical values).

The term “and/or” as used herein refers to multiple components in combination with or exclusive of one another. For example, “x, y, and/or z” can refer to “x” alone, “y” alone, “z” alone, “x, y, and z”, “(x and y) or z”, “(x and z) or y”, “(y and z) or x”, “x and y” alone, “x and z” alone, “y and z” alone, or “x or y or z”.

The terms “at least” and “one or more” as used herein refer to one, two, three, four, five, six, seven, eight, nine, ten, at least one, at least two, at least three, at least four, at least five, at least six, at least seven, at least eight, at least nine, at least ten, or more, or all of the elements subsequently listed.

The term “comparable composition”, “comparable food product”, “comparable batter/dough”, or “comparable baked/cooked food product” as used herein refer to a composition, food product, batter/dough, or baked/cooked food product, respectively, that is produced by a method that is identical to the method used to produce the composition, food product, batter/dough, or baked/cooked food product, respectively, that is compared to the “comparable composition”, “comparable food product”, “comparable batter/dough”, or “comparable baked/cooked food product”, respectively, except that the “comparable composition”, “comparable food product”, “comparable batter/dough”, or “comparable baked/cooked food product”, respectively, comprises egg in place of the egg replacer provided herein.

The term “component” as used herein refers to one unit (e.g., one protein) or more than one unit (e.g., two or more proteins) that are grouped together unless specifically stated otherwise. The grouping is to be understood as only a symbolic grouping, and thus does not require physical interaction between the units (although physical interaction is not ruled out by the use of the term “component”).

The term “egg” as used herein refers to an avian egg (i.e., an egg laid by a bird or fowl, such as, for example, chicken, quail, duck, ostrich, turkey, bantam, goose), or any component thereof. The term as used herein can refer to whole egg (including liquid whole egg, powdered (e.g., spray-dried, free-flowing dried (e.g., whole egg with <2% sodium silicoaluminate)) whole egg, frozen whole egg, salted whole egg (e.g., 10% salted whole egg (i.e., 10% salt and 90% whole egg)), sugared whole egg (e.g., 10% sugared whole egg (i.e., 10% sugar and 90% whole egg))), egg white (including liquid egg white, powdered (e.g., spray-dried, pan-dried) egg white, frozen egg white, stabilized egg white (e.g., lactic acid-aluminum sulfate-stabilized egg white, pH-adjusted egg white), whipping-aid egg white (e.g., egg white with 0.15% citric acid and 0.03% triethylcitrate)), or egg yolk (including liquid egg yolk, powdered (e.g., spray-dried, free-flowing dried (e.g., egg yolk with <2% sodium silicoaluminate)) egg yolk, frozen egg yolk, salted egg yolk (e.g., 10% salted egg yolk (i.e., 10% salt and 90% egg yolk)), sugared egg yolk (e.g., 10% sugared egg yolk (i.e., 10% sugar and 90% egg yolk))). Typical comparisons disclosed herein are made with large whole chicken egg (one large whole egg weighing about 50 g, not including the shell).

The term “egg attribute” as used herein refers to an attribute of whole egg, egg white, and/or egg yolk; or to an attribute that is imparted onto a composition (e.g., a food product) by whole egg, egg white, and/or egg yolk.

The term “egg protein” as used herein refers to a polypeptide that comprises a sequence of at least 20 (e.g., at least 20, at least 30, at least 40, at least 50, at least 60, at least 70, at least 80, at least 90, at least 100, at least 150) amino acids that is at least 50% (e.g., at least 50%, at least 55%, at least 60%, at least 65%, at least 70%, at least 75%, at least 80%, at least 85%, at least 90%, at least 95%, at least 99%, 100%) identical to a sequence of amino acids in a protein natively found in an egg (i.e., a protein that is native to egg). Non-limiting examples of egg proteins include ovalbumin, ovotransferrin (conalbumin), ovomucoid, G162M F167A ovomucoid, ovoglobulin G2, ovoglobulin G3, α-ovomucin, β-ovomucin, lysozyme, ovoinhibitor, ovoglycoprotein, flavoprotein, ovomacroglobulin, ovostatin, cystatin, avidin, ovalbumin related protein X, and ovalbumin related protein Y.

The term “egg replacer” as used herein refers to a composition that can be used as a substitute for (i.e., in place of) egg and that has at least one egg attribute.

The term “essentially free of” as used herein refers to the indicated component being either not detectable in the indicated composition by common analytical methods, or to the indicated component being present in such trace amounts as to not be functional. The term “functional” as used in this context refers to not contributing to properties of the composition comprising the trace amounts of the indicated component, or to not having activity (e.g., enzymatic activity) in the indicated composition comprising the trace amounts of the indicated component, or to not having health-adverse effects upon consumption of the composition comprising the trace amounts of the indicated component.

The term “filamentous fungus” as used herein refers to a cell from any filamentous form of the subdivision Eumycota and Oomycota (as defined by Hawksworth et al., In, Ainsworth and Bisby's Dictionary of The Fungi, 8th edition, 1995, CAB International, University Press, Cambridge, UK). A filamentous fungus is distinguished from yeast by its hyphal elongation during vegetative growth. The term “filamentous fungal cell” as used herein refers to a cell that is obtained from a filamentous fungus.

The term “food product” as used herein refers to a composition that can be ingested by a human or an animal for dietary purposes (i.e., without ill health effects but with significant nutritional and/or caloric intake due to uptake of digested material in the gastrointestinal tract), including a domesticated animal (e.g., dog, cat), farm animal (e.g., cow, pig, horse), and wild animal (e.g., non-domesticated predatory animal). The term includes compositions that can be combined with or added to one or more other ingredients to make a food product that can be ingested by a human or an animal.

The term “fungus” as used herein refers to organisms of the phyla Ascomycotas, Basidiomycota, Zygomycota, and Chythridiomycota, Oomycota, and Glomeromycota. It is understood, however, that fungal taxonomy is continually evolving, and therefore this specific definition of the fungal kingdom may be adjusted in the future. The term “fungal cell” as used herein refers to a cell that is obtained from a fungus.

The term “host cell” as used herein refers not only to the particular subject cell but to the progeny of such cell. Because certain modifications may occur in succeeding generations due to either mutation or environmental influences, such progeny may not, in fact, be identical to the parent cell, but are still included within the scope of the term “host cell” as used herein.

The term “identical” as used herein in the context of polynucleotide or polypeptide sequences refers to the residues in the two sequences that are the same when aligned for maximum correspondence. There are a number of different algorithms known in the art that can be used to measure polynucleotide or polypeptide sequence identity. For instance, sequences can be compared using FASTA (e.g., using its default parameters as provided in the Wisconsin Package Version 10.0, Genetics Computer Group (GCG), Madison, Wis.), Gap (e.g., using its default parameters as provided in the Wisconsin Package Version 10.0, GCG, Madison, Wis.), Bestfit, ClustalW (e.g., using default parameters of Version 1.83), or BLAST (e.g., using reciprocal BLAST, PSI-BLAST, BLASTP, BLASTN) (see, for example, Pearson. 1990. Methods Enzymol. 183:63; Altschul et al. 1990. J. Mol. Biol. 215:403).

The terms “including,” “includes,” “having,” “has,” “with,” or variants thereof as used herein are intended to be inclusive in a manner similar to the term “comprising”.

The term “materially contribute” as used herein refers to the indicated component contributing to an attribute of a composition to such extent that in the absence of the component (e.g., in a reference composition that is identical to the composition except that it lacks the indicated component) the attribute is at least 10%, at least 20%, at least 30%, at least 40%, or at least 50% less present/active/measurable.

The term “milk protein” as used herein refers to a polypeptide that comprises a sequence of at least 20 (e.g., at least 20, at least 30, at least 40, at least 50, at least 60, at least 70, at least 80, at least 90, at least 100, at least 150) amino acids that is at least 50% (e.g., at least 50%, at least 55%, at least 60%, at least 65%, at least 70%, at least 75%, at least 80%, at least 85%, at least 90%, at least 95%, at least 99%, 100%) identical to a sequence of amino acids in a protein natively found in a mammal-produced milk (i.e., a protein that is native to a mammal-produced milk; e.g., a native whey protein or a native casein). Non-limiting examples of milk proteins include α-lactalbumin (e.g., amino acids 20-142 of UniProt sequence P00709, P00711, P00712, or P09462), β-lactoglobulin (amino acids 17-178 of UniProt sequence P02754, amino acids 19-180 of UniProt sequence P67976 or P02756), lactoferrin (e.g., amino acids 20 to 708 of UniProt sequence P24627, D3G9G3, or Q29477; amino acids 20 to 710 of UniProt sequence P02788), transferrin (e.g., amino acids 20 to 704 of UniProt sequence Q29443, or W5PF65; amino acids 20 to 698 of UniProt sequence A0A452FJF9 or P02787), serum albumin (e.g., amino acids 25 to 607 of UniProt sequence P02769 or P14639; amino acids 19 to 608 of UniProt sequence A0A452F7Y5; amino acids 25 to 609 of UniProt sequence P02768), lactoperoxidase, glycomacropeptide (GMP), β-casein (e.g., amino acids 16 to 224 of UniProt sequence P02666; amino acids 16 to 222 of UniProt sequence P11839 or P33048; amino acids 16 to 226 of P05814), γ-casein, κ-casein (e.g., amino acids 22 to 190 of UniProt sequence P02668; amino acids 22 to 192 of UniProt sequence P02669 or P02670; amino acids 21 to 182 of UniProt sequence P07498), α-S1-casein (e.g., amino acids 16 to 214 of UniProt sequence P02662, P04653, or P18626; amino acids 16 to 185 of UniProt sequence P47710), and α-S2-casein (e.g., amino acids 16 to 222 of UniProt sequence P02663; amino acids 16 to 223 of UniProt sequence P04654 or P33049). Non-limiting examples of polynucleotide and polypeptide sequences encoding milk proteins are disclosed in PCT filing PCT/US2015/046428 filed Aug. 21, 2015, and PCT filing PCT/US2017/48730 filed Aug. 25, 2017, which are hereby incorporated herein, in their entireties.

The term “native” as used herein refers to what is produced in nature (e.g., produced by a cell that is not genetically modified by a human, and that is maintained under conditions [e.g., level of oxygenation, pH, salt concentration, temperature, and nutrient (e.g., carbon, nitrogen, sulfur) availability] that are not defined by a human).

The terms “optional” or “optionally” as used herein refer to a feature or structure being present or not, or an event or circumstance occurring or not. The description includes instances in which a feature or structure is present, instances in which the feature or structure is absent, instances in which an event or circumstance occurs, and instances in which an event or circumstance does not occur.

The term “polynucleotide” as used herein refers to both sense and antisense strands of RNA, cDNA, genomic DNA, and synthetic forms and mixed polymers of the above. A polynucleotide may be modified chemically or biochemically or may contain non-natural or derivatized nucleotide bases. Such modifications include, for example, labels; methylation; substitution of one or more of the naturally occurring nucleotides with an analog; internucleotide modifications such as uncharged linkages (e.g., methyl phosphonates, phosphotriesters, phosphoramidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Examples of modified nucleotides are described in the art (see, for example, Malyshev et al. 2014. Nature 509:385; Li et al. 2014. J. Am. Chem. Soc. 136:826). Also included are synthetic molecules that mimic polynucleotides in their ability to bind to a designated sequence via hydrogen bonding or other chemical interaction. Such molecules are known in the art and include, for example, molecules in which peptide linkages substitute for phosphate linkages in the backbone of the molecule. Other modifications can include, for example, analogs in which the ribose ring contains a bridging moiety or other structure such as the modifications found in “locked” polynucleotides.

The term “pH and/or ionic strength adjusting agent” as used herein refers to an agent that raises or lowers the pH and/or the ionic strength of a solution.

The term “polypeptide” as used herein refers to a polymeric form of amino acids.

The term “protein” as used herein refers to a polypeptide of any length, which can include polypeptides comprising coded and non-coded amino acids, polypeptides comprising amino acids that occur in nature and those that do not occur in nature, polypeptides comprising chemically or biochemically modified or derivatized amino acids, and polypeptides comprising modified peptide backbones.

The term “purifying” as used herein refers to a protein being substantially separated from chemicals and cellular components (e.g., cell walls, membrane lipids, chromosomes, other proteins). The term does not require (albeit allows) that the protein be separated from all other chemicals and cellular components.

The term “recombinant” as used herein in reference to a protein (e.g., a milk protein) refers to a protein that is produced recombinantly (e.g., that is produced by a recombinant host cell).

The term “recombinant host cell” as used herein refers to a host cell that comprises a recombinant polynucleotide. Thus, for example, a recombinant host cell may produce a polynucleotide or polypeptide not found in the native (non-recombinant) form of the host cell, or a recombinant host cell may produce a polynucleotide or polypeptide at a level that is different from that in the native (non-recombinant) form of the host cell. It should be understood that such term is intended to refer not only to the particular subject cell but also to the progeny of such a cell. Because certain modifications may occur in succeeding generations due to either mutation or environmental influences, such progeny may not be identical to the parent cell, but are still included within the scope of the term “recombinant host cell” as used herein.

The term “recombinant polynucleotide” as used herein refers to a polynucleotide that has been removed from its naturally occurring environment, a polynucleotide that is not associated with all or a portion of a polynucleotide abutting or proximal to the polynucleotide when it is found in nature, a polynucleotide that is operatively linked to a polynucleotide that it is not linked to in nature, a polynucleotide that is altered, or a polynucleotide that does not occur in nature. The term can be used, e.g., to describe cloned DNA isolates, or a polynucleotide comprising a chemically synthesized nucleotide analog. A polynucleotide is also considered “recombinant” if it contains a genetic modification that does not naturally occur. For instance, an endogenous polynucleotide is considered a “recombinant polynucleotide” if it contains an insertion, deletion, or substitution of one or more nucleotides that is introduced artificially (e.g., by human intervention). Such modification can introduce into the polynucleotide a point mutation, substitution mutation, deletion mutation, insertion mutation, missense mutation, frameshift mutation, duplication mutation, amplification mutation, translocation mutation, or inversion mutation. The term includes a polynucleotide in a host cell's chromosome, as well as a polynucleotide that is not in a host cell's chromosome (e.g., a polynucleotide that is comprised in an episome). A recombinant polynucleotide in a host cell or organism may replicate using the in vivo cellular machinery of the host cell; however, such recombinant polynucleotide, although subsequently replicated intracellularly, is still considered recombinant for purposes of this invention.

The term “whey protein” or “casein” as used herein refers to a polypeptide that comprises a sequence of at least 20 (e.g., at least 20, at least 30, at least 40, at least 50, at least 60, at least 70, at least 80, at least 90, at least 100, at least 150) amino acids that is at least 50% (e.g., at least 50%, at least 55%, at least 60%, at least 65%, at least 70%, at least 75%, at least 80%, at least 85%, at least 90%, at least 95%, at least 99%, 100%) identical to a sequence of amino acids in a native whey protein or casein, respectively. Non-limiting examples of whey proteins and caseins include: α-lactalbumin, β-lactoglobulin, lactoferrin, transferrin, serum albumin, lactoperoxidase, glycomacropeptide (GMP), β-casein, γ-casein, κ-casein, α-S1-casein, and α-S2-casein. Non-limiting examples of polynucleotide and polypeptide sequences encoding whey proteins and caseins are disclosed in PCT filing PCT/US2015/046428 filed Aug. 21, 2015, and PCT filing PCT/US2017/48730 filed Aug. 25, 2017, which are hereby incorporated herein, in their entireties.

The term “yeast” as used herein refers to organisms of the order Saccharomycetales. Vegetative growth of yeast is by budding/blebbing of a unicellular thallus, and carbon catabolism may be fermentative. The term “yeast cell” as used herein refers to a cell that is obtained from a yeast.

The term “% by mass” as used herein refers to a percentage value for a mass as determined in a hydrated composition, such that the composition includes the mass of powder as well as the mass of the hydrating agent, with 100% fixed as the percentage value for the entire hydrated composition. In embodiments in which the egg replacer provided herein is in powder form (to which the mass of the hydrating agent will be added at a later time), the term refers to a percentage value for a mass as determined relative to the eventual entire hydrated composition (with 100% fixed as the percentage value for that entire eventual hydrated composition).

The term “% by dry mass” as used herein in context of a hydrated composition (i.e., a composition comprising water) refers to a percentage value for a mass as determined relative to the total dry mass that would remain if the hydrated composition were to have substantially all water removed, with 100% fixed as the percentage value for that total dry mass. In context of a dry composition (e.g., a powder), the term “% by dry mass” as used herein refers to a percentage value for a mass as determined relative to the total mass of the powdered composition, with 100% fixed as the percentage value for that total dry mass.

Recitation of ranges of values herein are merely intended to serve as a shorthand method of referring individually to each separate value (fractional or integral) falling within the range inclusive of the recited minimum and maximum value, unless otherwise indicated herein, and each separate value is incorporated into the specification as if it were individually recited herein. Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of less than or equal to 10. It should further be understood that all ranges and quantities described below are approximations and are not intended to limit the invention. Where ranges and numbers are used these can be approximate to include statistical ranges or measurement errors or variation. In some embodiments, for instance, measurements could be plus or minus 10%.

### Egg Replacer Comprising Milk Protein Component

In one aspect, provided herein is an egg replacer that comprises a milk protein component, wherein the milk protein component imparts or materially contributes to an egg attribute of the egg replacer.

The term “milk protein component” as used herein refers to a component that consists of a subset of whey proteins or of a subset of caseins or of a mixture of a subset of whey proteins and a subset of caseins (i.e., consists of some but not all proteins present in, for example, whey protein concentrate, whey protein isolate, whey protein hydrolysate, casein isolate, casein concentrate, casein hydrolysate, milk protein isolate, milk protein concentrate, milk protein hydrolysate, micellar casein concentrate, sodium caseinate, or acid caseinate). The term implies that the milk proteins of which the milk protein component consists are the only milk proteins comprised in the egg replacer provided herein (i.e., the egg replacer comprises no milk proteins other than the milk proteins of which the milk protein component consists).

The egg replacer provided herein is desirable as it provides advantages in production, including but not limited to: independence from the productivity of egg-laying hens; independence from market uncertainties due to outbreaks of disease among livestock; more limited negative impact on the environment (e.g., smaller natural resources requirements [e.g., less water, land, energy used], lower carbon dioxide production); no negative impact on animal welfare (e.g., no animal confinement, force feeding, or hormone treatment); mitigation of supply chain and production risk (e.g., use of recombinant proteins obtained providing supply chain variations and increased flexibility in production methods); and lower production costs.

The egg replacer provided herein is further desirable as it provides advantages in composition and use, including but not limited to: being devoid of or having reduced levels of unhealthy components of animal egg (e.g., egg allergens, saturated fats, cholesterol, microbial contaminants [e.g., Salmonella, E. coli bacteria, bird viruses], avidin, phosvitin); not comprising non-Kosher contaminants (e.g., blood, embryo); being suitable for halal diet; being suitable for vegan diet; having similar or superior nutrient content as egg (e.g., favorable amino acid profiles, low sugar); having superior taste/flavor and aroma than egg; potential for being specifically engineered to have desirable or novel attributes; having a color that permits formulation of non-eggy colored food products (e.g., white vanilla cake) without the use of whitening agents or other ingredients (e.g., lecithin, sodium caseinate); having a dried form that can be stored at ambient conditions for an extended period of time (e.g., at least 1 year) without significant degradation or bacterial activity; having a readily reconstituted liquid or frozen form with extended shelf life; and/or providing functionality in replacing egg in a number of applications.

The egg replacer provided herein can be used for various purposes, including, for example, as a substitute for egg in an emulsion (e.g., any of the emulsions provided herein), as a substitute for egg in a batter (e.g., any of the batters provided herein), as a substitute for egg in a dough (e.g., any of the doughs provided herein), as a substitute for egg in a baked/cooked food product (e.g., any of the baked/cooked food products provided herein), or as a substitute for egg in an egg-based food product (e.g., any of the egg-based food products provided herein).

The egg replacer according to any of the above upon heating can comprise a milk protein polymer network. The term “milk protein polymer network” as used herein refers to a network that is composed of repeated milk protein units that are linked to each other via covalent or non-covalent (e.g., ionic) bonds, either directly with each other or via intermediary molecules. In some embodiments, the repeated milk protein units are linked to each other via disulfide bonds.

The egg replacer according to any of the above can be essentially free of gluten (i.e., comprises gluten less than 20 ppm), or can comprise less than 0.3%, less than 0.2%, less than 0.1%, less than 0.05%, or less than 0.01% by dry mass of gluten.

The egg replacer according to any of the above can be essentially free of a crosslinking agents (e.g., chemical crosslinking agents, enzymatic crosslinking agents), including, for example, enzymatic crosslinking agents. Such enzymatic crosslinking agents can be a transglutaminase (i.e., enzymes that catalyze the acyl-transfer between γ-carboxyamide groups and various primary amines, classified as EC 2.3.2.13).

The egg replacer according to any of the above can be essentially free of a gelling agent, such as, for example, a gelling agent selected from the group consisting of agar-agar, pectin, carrageenans, eucheuma seaweed, and modified cellulose (e.g., methylcellulose, methoxymethylcellulose, hydroxypropyl methylcellulose, and salts thereof).

The egg replacer according to any of the above can be essentially free of a protein obtained from plant (e.g., any of the plants disclosed herein), or comprise less than 0.5%, less than 1%, less than 5%, less than 10%, less than 15%, less than 20%, less than 30%, less than 40%, or less than 50% by dry mass of a protein obtained from plant.

The egg replacer according to any of the above can comprise no other protein other than the milk protein comprised in the milk protein component.

The egg replacer according to any of the above can be essentially free of an egg protein (e.g., one or more of ovomucoid, ovalbumin, ovotransferrin, G162M F167A ovomucoid, ovoglobulin G2, ovoglobulin G3, α-ovomucin, β-ovomucin, lysozyme, ovoinhibitor, ovoglycoprotein, flavoprotein, ovomacroglobulin, ovostatin, cystatin, avidin, ovalbumin related protein X, and ovalbumin related protein Y).

The egg replacer according to any of the above can be essentially free of one or more components obtained from an animal (i.e., components that are native to an animal, including animal products [i.e., parts of an animal that are consumables or typically prepared for consumption by humans; e.g., animal meat, animal fat, animal blood], animal byproducts [i.e., products that are typically not consumable by themselves but are the byproducts of slaughtering animals for consumption; e.g., animal bones, animal carcasses, and constituents isolated therefrom], products produced by an animal [e.g., mammal-derived milk, chicken eggs, bee honey], and consumables produced therefrom [e.g., gelatin, rennet, whey proteins extracted from mammal-derived milk, casein extracted from mammal-derived milk]), or comprise 2% or less by mass of a component obtained from an animal.

The egg replacer according to any of the above can be essentially free of a saturated lipid, or comprise less than 3%, less than 2%, less than 1%, less than 0.5%, or less than 0.1% by mass of a saturated lipid.

The egg replacer according to any of the above can be essentially free of cholesterol, or comprise less than 1.5%, less than 1%, less than 0.5%, less than 0.1%, or less than 0.05% by mass of cholesterol.

The egg replacer according to any of the above can be essentially free of lactose.

The egg replacer according to any of the above can have an allergenicity that is lower than that of egg, such as, for example, an allergenicity of up to 0%, 1%, 5%, 10%, 15%, 20%, 25%, 30%, 35%, 40%, 45%, 50%, 55%, 60%, 65%, 70%, 75%, 80%, 85%, 90%, or 95% relative to that of egg. Allergenicity may be measured using a skin prick test, blood test, or oral food challenge.

At standard ambient temperature and conditions (i.e., 20-30° C. and 0.95-1.05 atm), the egg replacer according to any of the above can be a fluid, semi-solid (e.g., gelatinous), solid, or powder. The egg replacer can be used in powder form, or the powder form can be reconstituted with a hydrating agent prior to use, or the powder form can be mixed with other dry components (e.g., flour, sugar, minerals, pH or ionic strength adjusting agents) before a hydrating agent is added to the mixture. Non-limiting examples of suitable hydrating agents include water, milk (e.g., animal milk, nut milk, plant-based milk), juice (e.g., vegetable juice, fruit juice, other plant juice), brine (e.g., fluid or liquid used to soak beans or legumes), and mixtures thereof.

### Milk Protein Component

The egg replacer according to any of the above can comprise between 0.1% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, or 0.2%; between 0.2% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, or 0.3%; between 0.3% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, or 0.4%; between 0.4% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, or 0.5%; between 0.5% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, or 0.6%; between 0.6% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, or 0.7%; between 0.7% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, or 0.8%; between 0.8% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, or 0.9%; between 0.9% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, or 1%; between 1% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, or 2%; between 2% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, or 3%; between 3% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, or 4%; between 4% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, or 5%; between 5% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, or 6%; between 6% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, or 7%; between 7% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, or 8%; between 8% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, or 9%; between 9% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, or 10%; between 10% and 100%, 95%, 90%, 85%, 80%, 75%, 70%, 65%, 60%, 55%, 50%, 45%, 40%, 35%, 30%, 25%, 20%, 15%, 14%, 13%, 12%, or 11%; between 11% and 100%, 95%, 90%, 85%, 80%, 75%, 70%, 65%, 60%, 55%, 50%, 45%, 40%, 35%, 30%, 25%, 20%, 15%, 14%, 13%, or 12%; between 12% and 100%, 95%, 90%, 85%, 80%, 75%, 70%, 65%, 60%, 55%, 50%, 45%, 40%, 35%, 30%, 25%, 20%, 15%, 14%, or 13%; between 13% and 100%, 95%, 90%, 85%, 80%, 75%, 70%, 65%, 60%, 55%, 50%, 45%, 40%, 35%, 30%, 25%, 20%, 15%, or 14%; between 14% and 100%, 95%, 90%, 85%, 80%, 75%, 70%, 65%, 60%, 55%, 50%, 45%, 40%, 35%, 30%, 25%, 20%, or 15%; between 15% and 100%, 95%, 90%, 85%, 80%, 75%, 70%, 65%, 60%, 55%, 50%, 45%, 40%, 35%, 30%, 25%, or 20%; between 20% and 100%, 95%, 90%, 85%, 80%, 75%, 70%, 65%, 60%, 55%, 50%, 45%, 40%, 35%, 30%, or 25%; between 25% and 100%, 95%, 90%, 85%, 80%, 75%, 70%, 65%, 60%, 55%, 50%, 45%, 40%, 35%, or 30%; between 30% and 100%, 95%, 90%, 85%, 80%, 75%, 70%, 65%, 60%, 55%, 50%, 45%, 40%, or 35%; between 35% and 100%, 95%, 90%, 85%, 80%, 75%, 70%, 65%, 60%, 55%, 50%, 45%, or 40%; between 40% and 100%, 95%, 90%, 85%, 80%, 75%, 70%, 65%, 60%, 55%, 50%, or 45%; between 45% and 100%, 95%, 90%, 85%, 80%, 75%, 70%, 65%, 60%, 55%, or 50%; between 50% and 100%, 95%, 90%, 85%, 80%, 75%, 70%, 65%, 60%, or 55%; between 55% and 100%, 95%, 90%, 85%, 80%, 75%, 70%, 65%, or 60%; between 60% and 100%, 95%, 90%, 85%, 80%, 75%, 70%, or 65%; between 65% and 100%, 95%, 90%, 85%, 80%, 75%, or 70%; between 70% and 100%, 95%, 90%, 85%, 80%, or 75%; between 75% and 100%, 95%, 90%, 85%, or 80%; between 80% and 100%, 95%, 90%, or 85%; or between 85% and 100%, 95%, 90%; between 90% and 100% or 95%, or between 95% and 100% by mass of the milk protein component.

The milk protein component of the egg replacer according to any of the above can consist of: one or more whey proteins, one or more caseins, or a mixture of one or more whey proteins and one or more caseins. Non-limiting examples of whey proteins and caseins include: β-lactoglobulin, α-lactalbumin, lactoferrin, transferrin, serum albumin, lactoperoxidase, GMP, β-casein, γ-casein, κ-casein, α-S1-casein, and α-S2-casein. Non-limiting examples of mixtures of whey proteins, caseins, or whey proteins and caseins include: β-lactoglobulin combined with any one or more of α-lactalbumin, lactoferrin, transferrin, serum albumin, lactoperoxidase, GMP, β-casein, γ-casein, κ-casein, α-S1-casein, and α-S2-casein; α-lactalbumin combined with any one or more of lactoferrin, transferrin, serum albumin, lactoperoxidase, GMP, β-casein, γ-casein, κ-casein, α-S1-casein, and α-S2-casein; lactoferrin combined with any one or more of transferrin, serum albumin, lactoperoxidase, GMP, β-casein, γ-casein, κ-casein, α-S1-casein, and α-S2-casein; transferrin combined with any one or more of serum albumin, lactoperoxidase, GMP, β-casein, γ-casein, κ-casein, α-S1-casein, and α-S2-casein; serum albumin combined with any one or more of lactoperoxidase, GMP, β-casein, γ-casein, κ-casein, α-S1-casein, and α-S2-casein; lactoperoxidase combined with any one or more of GMP, β-casein, γ-casein, κ-casein, α-S1-casein, and α-S2-casein; GMP combined with any one or more of β-casein, γ-casein, κ-casein, α-S1-casein, and α-S2-casein; β-casein combined with any one or more of γ-casein, κ-casein, α-S1-casein, and α-S2-casein; γ-casein combined with any one or more of κ-casein, α-S1-casein, and α-S2-casein; κ-casein combined with one or both of α-S1-casein and α-S2-casein; α-S1-casein combined with α-S2-casein.

The milk protein component of the egg replacer according to any of the above can consist of: one or more native milk proteins (e.g., a native whey protein, a native casein), one or more recombinant milk proteins (e.g., a recombinant whey protein, a recombinant casein), or a mixture of one or more native milk proteins and one or more recombinant milk proteins. Non-limiting examples of native and recombinant milk proteins include: native β-lactoglobulin, native α-lactalbumin, native lactoferrin, native transferrin, native serum albumin, native lactoperoxidase, native GMP, native β-casein, native γ-casein, native κ-casein, native α-S1-casein, and native α-S2-casein, recombinant β-lactoglobulin, recombinant α-lactalbumin, recombinant lactoferrin, recombinant transferrin, recombinant serum albumin, recombinant lactoperoxidase, recombinant GMP, recombinant β-casein, recombinant γ-casein, recombinant κ-casein, recombinant α-S1-casein, and recombinant α-S2-casein.

The milk protein component of the egg replacer according to any of the above can consist of whey protein and casein at a mass ratio of between about 10 to 1 and about 1 to 10 (e.g., about 10 to 1, about 9 to 1, about 8 to 1, about 7 to 1, about 6 to 1, about 5 to 1, about 4 to 1, about 3 to 1, about 2 to 1, about 1 to 1, about 1 to 2, about 1 to 3, about 1 to 4, about 1 to 5, about 1 to 6, about 1 to 7, about 1 to 8, about 1 to 9, about 1 to 10).

The milk protein component of the egg replacer according to any of the above can comprise or consist of native whey protein and native casein at a mass ratio of between about 10 to 1 and about 1 to 10 (e.g., about 10 to 1, about 9 to 1, about 8 to 1, about 7 to 1, about 6 to 1, about 5 to 1, about 4 to 1, about 3 to 1, about 2 to 1, about 1 to 1, about 1 to 2, about 1 to 3, about 1 to 4, about 1 to 5, about 1 to 6, about 1 to 7, about 1 to 8, about 1 to 9, about 1 to 10).

The milk protein component of the egg replacer according to any of the above can comprise or consist of recombinant whey protein and recombinant casein at a mass ratio of between about 10 to 1 and about 1 to 10 (e.g., about 10 to 1, about 9 to 1, about 8 to 1, about 7 to 1, about 6 to 1, about 5 to 1, about 4 to 1, about 3 to 1, about 2 to 1, about 1 to 1, about 1 to 2, about 1 to 3, about 1 to 4, about 1 to 5, about 1 to 6, about 1 to 7, about 1 to 8, about 1 to 9, about 1 to 10).

The milk protein component of the egg replacer according to any of the above can comprise or consist of native whey protein and recombinant casein at a mass ratio of between about 10 to 1 and about 1 to 10 (e.g., about 10 to 1, about 9 to 1, about 8 to 1, about 7 to 1, about 6 to 1, about 5 to 1, about 4 to 1, about 3 to 1, about 2 to 1, about 1 to 1, about 1 to 2, about 1 to 3, about 1 to 4, about 1 to 5, about 1 to 6, about 1 to 7, about 1 to 8, about 1 to 9, about 1 to 10).

The milk protein component of the egg replacer according to any of the above can comprise or consist of recombinant whey protein and native casein at a mass ratio of between about 10 to 1 and about 1 to 10 (e.g., about 10 to 1, about 9 to 1, about 8 to 1, about 7 to 1, about 6 to 1, about 5 to 1, about 4 to 1, about 3 to 1, about 2 to 1, about 1 to 1, about 1 to 2, about 1 to 3, about 1 to 4, about 1 to 5, about 1 to 6, about 1 to 7, about 1 to 8, about 1 to 9, about 1 to 10).

The milk protein component of the egg replacer according to any of the above can comprise or consist of recombinant milk proteins and native milk proteins at a mass ratio of between about 100 to 1 and about 1 to 100 (e.g., about 100 to 1, about 90 to 1, about 80 to 1, about 70 to 1, about 60 to 1, about 50 to 1, about 40 to 1, about 30 to 1, about 20 to 1, about 10 to 1, about 9 to 1, about 8 to 1, about 7 to 1, about 6 to 1, about 5 to 1, about 4 to 1, about 3 to 1, about 2 to 1, about 1 to 1, about 1 to 2, about 1 to 3, about 1 to 4, about 1 to 5, about 1 to 6, about 1 to 7, about 1 to 8, about 1 to 9, about 1 to 10, about 1 to 20, about 1 to 30, about 1 to 40, about 1 to 50, about 1 to 60, about 1 to 70, about 1 to 80, about 1 to 90, or about 1 to 100).

The milk protein component of the egg replacer according to any of the above can comprise or consist of recombinant whey protein and native whey protein at a mass ratio of between about 10 to 1 and about 1 to 10 (e.g., about 10 to 1, about 9 to 1, about 8 to 1, about 7 to 1, about 6 to 1, about 5 to 1, about 4 to 1, about 3 to 1, about 2 to 1, about 1 to 1, about 1 to 2, about 1 to 3, about 1 to 4, about 1 to 5, about 1 to 6, about 1 to 7, about 1 to 8, about 1 to 9, about 1 to 10).

The milk protein component of the egg replacer according to any of the above can comprise or consist of recombinant casein and native casein at a mass ratio of between about 10 to 1 and about 1 to 10 (e.g., about 10 to 1, about 9 to 1, about 8 to 1, about 7 to 1, about 6 to 1, about 5 to 1, about 4 to 1, about 3 to 1, about 2 to 1, about 1 to 1, about 1 to 2, about 1 to 3, about 1 to 4, about 1 to 5, about 1 to 6, about 1 to 7, about 1 to 8, about 1 to 9, about 1 to 10).

The native milk protein of the milk protein component of the egg replacer according to any of the above can be obtained from any mammalian species, including but not limited to cow, human, sheep, goat, water buffalo, camel, horse, donkey, lemur, panda, guinea pig, squirrel, bear, macaque, gorilla, chimpanzee, mountain goat, monkey, ape, cat, dog, wallaby, rat, mouse, elephant, opossum, rabbit, whale, baboons, gibbons, orangutan, mandrill, pig, wolf, fox, lion, tiger, reindeer, and echidna.

The milk protein component of the egg replacer according to any of the above can comprise a native and/or recombinant milk protein that lacks an allergenic epitope (i.e., an amino acid sequence that can elicit an immune response in a human or animal).

The milk protein component of the egg replacer according to any of the above can comprise a recombinant milk protein that comprises a reactive site. The term “reactive site” as used herein refers to a chemical group on a molecule (e.g., polypeptide) that can form a covalent bond (i.e., “crosslink”) with a chemical group on another molecule (e.g., another polypeptide). The reactive site can be a native reactive site, a non-native reactive site (i.e., a reactive site that is not present in the native milk protein), or a mixture of at least one native reactive site and at least one non-native reactive site. Non-limiting examples of suitable reactive sites include disulfides, thiols, primary amines, carboxyls, carboxamides, and hydroxyls (see, for example, (Means & Feeney. 1971. Chemical Modification of Proteins. Holden Day, Inc. San Francisco, Cambridge, London, Amsterdam). Methods for introducing or removing reactive sites in polypeptides are known in the art (see, for example, Means, G A & Feeney, R E. 1971. Chemical Modification of Proteins. Holden Day, Inc. San Francisco, Cambridge, London, Amsterdam), and include, for example, deleting or inserting amino acid residues comprising reactive sites (e.g., cysteine residues, lysine residues, glutamic acid residues, aspartic acid residues, non-natural amino acids (e.g., selenocysteine, methylated amino acids, argenylated amino acids, acylated amino acids, biotinylated amino acids)). The type and/or number and/or location of a reactive site in a recombinant milk protein can confer or materially impact an egg attribute in the egg replacer according to any of the above. It is also within the scope of the invention that a recombinant milk protein comprised in the milk protein component of the egg replacer according to any of the above lacks a reactive site that is present in the corresponding native milk protein.

The milk protein component of the egg replacer according to any of the above can comprise a recombinant milk protein that comprises a milk protein repeat. The term “milk protein repeat” as used herein refers to an amino acid sequence that is at least 80% identical (e.g., at least 80%, at least 85%, at least 90%, at least 95% identical, at least 99% identical) to an amino acid sequence in a protein found in a mammal-produced milk (e.g., a whey protein, a casein) and that is present more than once (e.g., at least 2, at least 3, at least 4, at least 5, at least 10, at least 15, at least 20, at least 30, at least 40, at least 50, at least 75, at least 100, at least 150, or at least 200 times) in the recombinant milk protein. A milk protein repeat may comprise at least 10, at least 20, at least 30, at least 40, at least 50, at least 75, at least 100, at least 150, and usually not more than 200 amino acids. A milk protein repeat in a recombinant milk protein can be consecutive (i.e., have no intervening amino acid sequences) or non-consecutive (i.e., have intervening amino acid sequences). When present non-consecutively, the intervening amino acid sequence may play a passive role in providing molecular weight without introducing an undesirable property, or may play an active role in providing for a particular property (e.g., solubility, biodegradability, binding to other molecules). The type and/or number and/or location of a milk protein repeat in a recombinant milk protein can confer or materially impact an egg attribute in the egg replacer according to any of the above.

The milk protein component of the egg replacer according to any of the above can comprise a recombinant milk protein that comprises a post-translational modification. The term “post-translational modification”, or its acronym “PTM”, as used herein refers to a modification to a protein after biosynthesis (e.g., the covalent attachment of a chemical group to a protein). PTM can occur on the amino acid side chain of the protein or at its C- or N-termini. Non-limiting examples of suitable PTMs include glycosylation (i.e., covalent attachment to proteins of glycan groups (e.g., monosaccharides, disaccharides, polysaccharides, linear glycans, branched glycans, glycans with galf residues, glycans with sulfate and/or phosphate residues, D-glucose, D-galactose, D-mannose, L-fucose, N-acetyl-D-galactose amine, N-acetyl-D-glucose amine, N-acetyl-D-neuraminic acid, galactofuranose, phosphodiesters, N-acetylglucosamine, N-acetylgalactosamine, sialic acid, and combinations thereof; see, for example, Deshpande et al. 2008. Glycobiology 18(8):626) via C-linkage (i.e., C-glycosylation), N-linkage (i.e., N-glycosylation), or 0-linkage (i.e., O-glycosylation), or via glypiation (i.e., addition of a glycosylphosphatidylinositol anchor) or phosphoglycosylation (i.e., linked through the phosphate of a phospho-serine)), phosphorylation (i.e., covalent attachment to proteins of phosphate groups), alkylation (i.e., covalent attachment to proteins of alkane groups (e.g., methane group in methylation)), lipidation (i.e., covalent attachment of a lipid group (e.g., isoprenoid group in prenylation and isoprenylation (e.g., farnesol group in farnesylation, geraniol group in geranylation, geranylgeraniol group in geranylgeranylation), fatty acid group in fatty acylation (e.g., myristic acid in myristoylation, palmitic acid in palmitoylation), glycosylphosphatidylinositol anchor in glypiation)), hydroxylation (i.e., covalent attachment of a hydroxide group), sumoylation (i.e., attachment to proteins of Small Ubiquitin-like Modifier (or SUMO) protein), nitrosylation (i.e., attachment to proteins of an NO group (e.g., S-nitrosylation)), nitrosothiolation (i. e., attachment to a cysteine thiol in a protein of an NO group (e.g., S-nitrosothiol)), glutathionylation (i.e., attachment to a cysteine thiol in a protein of a glutathione group (e.g., S-glutathionylation)), and tyrosine nitration (i.e., attachment to tyrosine residues of proteins of nitrate groups). PTMs of a recombinant milk protein can be mammalian PTMs, non-mammalian PTMs, or mixtures of at least one mammalian PTM and at least one non-mammalian PTM. The term “non-mammalian PTM” as used herein refers to a difference in one or more location(s) of one or more PTMs (e.g., glycosylation, phosphorylation) in a protein, and/or a difference in the type of one or more PTMs at one or more location(s) in a protein compared to the protein as it exists in a mammal-derived milk (i.e., the protein having “mammalian PTMs”). The type and/or number and/or absence of and/or location of PTMs in a recombinant milk protein can confer or materially impact an egg attribute in the egg replacer according to any of the above.

The milk protein component of the egg replacer according to any of the above can comprise between 5% and 100%, 99%, 95%, 90%, 85%, 80%, 75%, 70%, 65%, 60%, 55%, 50%, 45%, 40%, 35%, 30%, 25%, 20%, 15%, or 10%; between 10% and 100%, 99%, 95%, 90%, 85%, 80%, 75%, 70%, 65%, 60%, 55%, 50%, 45%, 40%, 35%, 30%, 25%, 20%, or 15%; between 15% and 100%, 99%, 95%, 90%, 85%, 80%, 75%, 70%, 65%, 60%, 55%, 50%, 45%, 40%, 35%, 30%, 25%, or 20%; between 20% and 100%, 99%, 95%, 90%, 85%, 80%, 75%, 70%, 65%, 60%, 55%, 50%, 45%, 40%, 35%, 30%, or 25%; between 25% and 100%, 99%, 95%, 90%, 85%, 80%, 75%, 70%, 65%, 60%, 55%, 50%, 45%, 40%, 35%, or 30%; between 30% and 100%, 99%, 95%, 90%, 85%, 80%, 75%, 70%, 65%, 60%, 55%, 50%, 45%, 40%, or 35%; between 35% and 100%, 99%, 95%, 90%, 85%, 80%, 75%, 70%, 65%, 60%, 55%, 50%, 45%, or 40%; between 40% and 100%, 99%, 95%, 90%, 85%, 80%, 75%, 70%, 65%, 60%, 55%, 50%, or 45%; between 45% and 100%, 99%, 95%, 90%, 85%, 80%, 75%, 70%, 65%, 60%, 55%, or 50%; between 50% and 100%, 99%, 95%, 90%, 85%, 80%, 75%, 70%, 65%, 60%, or 55%; between 55% and 100%, 99%, 95%, 90%, 85%, 80%, 75%, 70%, 65%, or 60%; between 60% and 100%, 99%, 95%, 90%, 85%, 80%, 75%, 70%, or 65%; between 65% and 100%, 99%, 95%, 90%, 85%, 80%, 75%, or 70%; between 70% and 100%, 99%, 95%, 90%, 85%, 80%, or 75%; between 75% and 100%, 99%, 95%, 90%, 85%, or 80%; between 80% and 100%, 99%, 95%, 90%, or 85%; between 85% and 100%, 99%, 95%, or 90%; between 90% and 100%, 99%, or 95%; between 95% and 100%, or 99%; or between 99% and 100% by dry mass of a recombinant β-lactoglobulin having one or more non-mammalian PTMs and/or lacking one or more mammalian PTMs, wherein the recombinant β-lactoglobulin imparts or materially contributes to an egg attribute of the egg replacer.

The milk protein component of the egg replacer according to any of the above can comprise between 5% and 100%, 99%, 95%, 90%, 85%, 80%, 75%, 70%, 65%, 60%, 55%, 50%, 45%, 40%, 35%, 30%, 25%, 20%, 15%, or 10%; between 10% and 100%, 99%, 95%, 90%, 85%, 80%, 75%, 70%, 65%, 60%, 55%, 50%, 45%, 40%, 35%, 30%, 25%, 20%, or 15%; between 15% and 100%, 99%, 95%, 90%, 85%, 80%, 75%, 70%, 65%, 60%, 55%, 50%, 45%, 40%, 35%, 30%, 25%, or 20%; between 20% and 100%, 99%, 95%, 90%, 85%, 80%, 75%, 70%, 65%, 60%, 55%, 50%, 45%, 40%, 35%, 30%, or 25%; between 25% and 100%, 99%, 95%, 90%, 85%, 80%, 75%, 70%, 65%, 60%, 55%, 50%, 45%, 40%, 35%, or 30%; between 30% and 100%, 99%, 95%, 90%, 85%, 80%, 75%, 70%, 65%, 60%, 55%, 50%, 45%, 40%, or 35%; between 35% and 100%, 99%, 95%, 90%, 85%, 80%, 75%, 70%, 65%, 60%, 55%, 50%, 45%, or 40%; between 40% and 100%, 99%, 95%, 90%, 85%, 80%, 75%, 70%, 65%, 60%, 55%, 50%, or 45%; between 45% and 100%, 99%, 95%, 90%, 85%, 80%, 75%, 70%, 65%, 60%, 55%, or 50%; between 50% and 100%, 99%, 95%, 90%, 85%, 80%, 75%, 70%, 65%, 60%, or 55%; between 55% and 100%, 99%, 95%, 90%, 85%, 80%, 75%, 70%, 65%, or 60%; between 60% and 100%, 99%, 95%, 90%, 85%, 80%, 75%, 70%, or 65%; between 65% and 100%, 99%, 95%, 90%, 85%, 80%, 75%, or 70%; between 70% and 100%, 99%, 95%, 90%, 85%, 80%, or 75%; between 75% and 100%, 99%, 95%, 90%, 85%, or 80%; between 80% and 100%, 99%, 95%, 90%, or 85%; between 85% and 100%, 99%, 95%, or 90%; between 90% and 100%, 99%, or 95%; between 95% and 100%, or 99%; or between 99% and 100% by dry mass of a recombinant α-lactalbumin having one or more non-mammalian PTMs and/or lacking one or more mammalian PTMs, wherein the recombinant α-lactalbumin imparts or materially contributes to an egg attribute of the egg replacer.

The milk protein component of the egg replacer according to any of the above can comprise between 5% and 100%, 99%, 95%, 90%, 85%, 80%, 75%, 70%, 65%, 60%, 55%, 50%, 45%, 40%, 35%, 30%, 25%, 20%, 15%, or 10%; between 10% and 100%, 99%, 95%, 90%, 85%, 80%, 75%, 70%, 65%, 60%, 55%, 50%, 45%, 40%, 35%, 30%, 25%, 20%, or 15%; between 15% and 100%, 99%, 95%, 90%, 85%, 80%, 75%, 70%, 65%, 60%, 55%, 50%, 45%, 40%, 35%, 30%, 25%, or 20%; between 20% and 100%, 99%, 95%, 90%, 85%, 80%, 75%, 70%, 65%, 60%, 55%, 50%, 45%, 40%, 35%, 30%, or 25%; between 25% and 100%, 99%, 95%, 90%, 85%, 80%, 75%, 70%, 65%, 60%, 55%, 50%, 45%, 40%, 35%, or 30%; between 30% and 100%, 99%, 95%, 90%, 85%, 80%, 75%, 70%, 65%, 60%, 55%, 50%, 45%, 40%, or 35%; between 35% and 100%, 99%, 95%, 90%, 85%, 80%, 75%, 70%, 65%, 60%, 55%, 50%, 45%, or 40%; between 40% and 100%, 99%, 95%, 90%, 85%, 80%, 75%, 70%, 65%, 60%, 55%, 50%, or 45%; between 45% and 100%, 99%, 95%, 90%, 85%, 80%, 75%, 70%, 65%, 60%, 55%, or 50%; between 50% and 100%, 99%, 95%, 90%, 85%, 80%, 75%, 70%, 65%, 60%, or 55%; between 55% and 100%, 99%, 95%, 90%, 85%, 80%, 75%, 70%, 65%, or 60%; between 60% and 100%, 99%, 95%, 90%, 85%, 80%, 75%, 70%, or 65%; between 65% and 100%, 99%, 95%, 90%, 85%, 80%, 75%, or 70%; between 70% and 100%, 99%, 95%, 90%, 85%, 80%, or 75%; between 75% and 100%, 99%, 95%, 90%, 85%, or 80%; between 80% and 100%, 99%, 95%, 90%, or 85%; between 85% and 100%, 99%, 95%, or 90%; between 90% and 100%, 99%, or 95%; between 95% and 100%, or 99%; or between 99% and 100% by dry mass of a recombinant lactoferrin, transferrin, serum albumin, lactoperoxidase, GMP, β-casein, γ-casein, κ-casein, α-S1-casein, or α-S2-casein, having one or more non-mammalian PTMs and/or lacking one or more mammalian PTMs, wherein the recombinant lactoferrin, transferrin, serum albumin, lactoperoxidase, GMP, β-casein, γ-casein, κ-casein, α-S1-casein, or α-S2-casein imparts or materially contributes to an egg attribute of the egg replacer.

### Carbohydrate

The egg replacer according to any of the above can further comprise a carbohydrate. Such egg replacer can have particular utility in producing an emulsion (e.g., any of the emulsions provided herein), a batter (e.g., any of the batters provided herein), a dough (e.g., any of the doughs provided herein), a baked/cooked food product (e.g., any of the baked/cooked food products provided herein), or an egg-based food product (e.g., any of the egg-based food products provided herein).

The carbohydrate can be obtained from any source, including, for example, a plant (e.g., any of the plants disclosed herein), an algae (e.g., any of the algae disclosed herein), a fungus (e.g., any of the fungi disclosed herein), or a bacterium (e.g., any of the bacteria disclosed herein).

Nonlimiting examples of suitable carbohydrates include: monosaccharides, such as, for example, glucose, fructose, galactose, and mixtures thereof; disaccharides, such as, for example, maltose, lactose, sucrose, and mixtures thereof; polysaccharides, such as for example, starches (e.g., pectin, corn (maize) starch, oat starch, potato starch, sweet potato starch, rice starch, pea starch, wheat starch, azuki starch, green bean starch, kudzu starch, Katakuri starch, arrowroot starch, mung bean starch, sago starch, tapioca starch, plant starch (e.g., starch obtained from any of the plants disclosed herein), and derivatives thereof, and mixtures of two or more thereof. In some embodiments, the starch is a modified starch (e.g., pregelatinized starch (e.g., corn, wheat, tapioca), pregelatinized high amylose content starch, pregelatinized hydrolyzed starches (e.g., maltodextrins, corn syrup solids, rice syrup solids, tapioca syrup solids), chemically modified starches such as pregelatinized substituted starches (e.g., octenyl succinate modified starches), alkaline modified starch, bleached starch, oxidized starch, monostarch phosphate, distarch phosphate, phosphated distarch phosphate, acetylated distarch phosphate, acetylated starch, mono starch acetate, acetylated starch, mono starch acetate, acetylated distarch adipate, distarch glycerine, hydroxy propyl starch, hydroxy propyl distarch glycerine, hydroxy propyl distarch phosphate, starch sodium octenyl succinate, acetylated oxidized starch, dextrin, sodium octenylsuccinate starch, and derivatives thereof, and mixtures of two or more thereof), flours (e.g., acorn flour, almond flour, amaranth flour, atta flour, barley flour, bean flour, buckwheat flour, cassava flour, chestnut flour, chuño flour, coconut flour, corn (maize) flour, durum flour, einkorn flour, emmer flour, fava bean flour, garbanzo flour, ground chia seeds, ground flaxseeds, hemp flour, khorasan flour, lentil flour, maida flour, malted barley flour, masa harina, mesquite flour, millet flour, nut flour, oat flour, pea flour, peanut flour, potato flour, quinoa flour, rice flour, rye flour, sorghum flour, soy flour, spelt flour, sweet rice flour, taro flour, teff flour, wheat flour, vital wheat gluten, ground chia seeds, ground flaxseed, and derivatives thereof, and mixtures of two or more thereof), gums (e.g., arrowroot flour, xanthan gum, acacia gum (gum arabic), gellan gum, guar gum, locust bean gum (carob gum), tragacanth gum, carrageenan, tara gum, wheat gum, konjac gum, agar gum, karaya gum, salep, modified cellulose (e.g., methylcellulose, methoxymethylcellulose, hydroxypropyl methylcellulose, carboxymethylcellulose, microcrystalline cellulose), and derivatives and mixtures thereof. In some embodiments, the gum is a modified gum (e.g., deacetylated, deacetylated clarified, partially deacetylated, partially deacetylated clarified, and derivatives thereof, and mixtures of two or more thereof)), edible fibers (e.g., acacia fiber, bamboo fiber, barley bran, carrot fiber, cellulose (e.g., wood pulp cellulose), citrus fiber, corn bran, soluble dietary fiber, insoluble dietary fiber, oat bran, pea fiber, rice bran, psyllium husk, konjac, soy fiber, soy polysaccharide, wheat bran, inulin, and derivatives thereof, and mixtures of two or more thereof), and mixtures of two or more thereof; and mixtures of two or more thereof.

The egg replacer according to any of the above can comprise between 0.05% and 40%, 35%, 30%, 25%, 20%, 15%, 10%, 5%, 1%, 0.5%, or 0.1%; between 0.1% and 40%, 35%, 30%, 25%, 20%, 15%, 10%, 5%, 1%, or 0.5%; between 0.5% and 40%, 35%, 30%, 25%, 20%, 15%, 10%, 5%, or 1%; between 1% and 40%, 35%, 30%, 25%, 20%, 15%, 10%, or 5%; between 5% and 40%, 35%, 30%, 25%, 20%, 15%, or 10%; between 10% and 40%, 35%, 30%, 25%, 20%, or 15%; between 15% and 40%, 35%, 30%, 25%, or 20%; between 20% and 40%, 35%, 30%, or 25%; between 25% and 40%, 35%, or 30%; between 30% and 40%, or 35%; or between 35% and 40% by mass of a starch (e.g., any of the starches disclosed herein, such as, for example, tapioca starch, corn (maize) starch, potato starch, arrowroot starch, tapioca syrup solids, or rice syrup solids, or any mixture of two or more thereof).

The egg replacer according to any of the above can comprise between 0.01% and 5%, 4.5%, 4%, 3.5%, 3%, 2.5%, 2%, 1.5%, 1%, 0.5%, 0.1%, or 0.05%; between 0.05% and 5%, 4.5%, 4%, 3.5%, 3%, 2.5%, 2%, 1.5%, 1%, 0.5%, or 0.1%; between 0.1% and 5%, 4.5%, 4%, 3.5%, 3%, 2.5%, 2%, 1.5%, 1%, or 0.5%; between 0.5% and 5%, 4.5%, 4%, 3.5%, 3%, 2.5%, 2%, 1.5%, or 1%; between 1% and 5%, 4.5%, 4%, 3.5%, 3%, 2.5%, 2%, or 1.5%; between 1.5% and 5%, 4.5%, 4%, 3.5%, 3%, 2.5%, or 2%; between 2% and 5%, 4.5%, 4%, 3.5%, 3%, or 2.5%; between 2.5% and 5%, 4.5%, 4%, 3.5%, or 3%; between 3% and 5%, 4.5%, 4%, or 3.5%; between 3.5% and 5%, 4.5%, or 4%; between 4% and 5%, or 4.5%; or between 4.5% and 5% by mass of a gum (e.g., any of the gums disclosed herein, such as, for example, arrowroot flour, xanthan gum, tara gum, guar gum, agar gum, locust bean gum, or gum arabic, or any mixture of two or more thereof).

The egg replacer according to any of the above can comprise between 0.1% and 20%, 15%, 10%, 5%, 1%, or 0.5%; between 0.5% and 20%, 15%, 10%, 5%, or 1%; between 1% and 20%, 15%, 10%, or 5%; between 5% and 20%, 15%, or 10%; between 10% and 20%, or 15%; or between 15% and 20% by mass of an edible fiber (e.g., any of the edible fibers disclosed herein, such as, for example, psyllium husk, acacia fiber, citrus fiber, or cellulose, or any mixture of two or more thereof).

The egg replacer according to any of the above can comprise between 0.1% and 30%, 25%, 20%, 15%, 10%, 5%, 1%, or 0.5%; between 0.5% and 30%, 25%, 20%, 15%, 10%, 5%, or 1%; between 1% and 30%, 25%, 20%, 15%, 10%, or 5%; between 5% and 30%, 25%, 20%, 15%, or 10%; between 10% and 30%, 25%, 20%, or 15%; between 15% and 30%, 25%, or 20%; between 20% and 30%, or 25%; or between 25% and 30% by mass of a flour (e.g., any of the flours disclosed herein, such as, for example, ground chia seeds, ground flaxseed, or sweet rice flour, or any mixture of two or more thereof).

The egg replacer according to any of the above can comprise between 0.05% and 40% by mass of a starch (e.g., any of the starches disclosed herein, such as, for example, tapioca starch, corn (maize) starch, potato starch, arrowroot starch, tapioca syrup solids, or rice syrup solids, or any mixture of two or more thereof), and between 0.01% and 5% by mass of a gum (e.g., any of the gums disclosed herein, such as, for example, arrowroot flour, xanthan gum, tara gum, guar gum, agar gum, locust bean gum, or gum arabic, or any mixture of two or more thereof). For example, the egg replacer according to any of the above can comprise between 0.05% and 40% by mass of a tapioca starch, and between 0.01% and 5% by mass of xantham gum (see Example 3).

The egg replacer according to any of the above can comprise between 0.05% and 40% by mass of a starch (e.g., any of the starches disclosed herein, such as, for example, tapioca starch, corn (maize) starch, potato starch, arrowroot starch, tapioca syrup solids, or rice syrup solids, or any mixture of two or more thereof), and between 0.1% and 20% by mass of an edible fiber (e.g., any of the edible fibers disclosed herein, such as, for example, psyllium husk, acacia fiber, citrus fiber, or cellulose, or any mixture of two or more thereof).

The egg replacer according to any of the above can comprise between 0.05% and 40% by mass of a starch (e.g., any of the starches disclosed herein, such as, for example, tapioca starch, corn (maize) starch, potato starch, arrowroot starch, tapioca syrup solids, or rice syrup solids, or any mixture of two or more thereof), and between 0.1% and 30% by mass of a flour (e.g., any of the flours disclosed herein, such as, for example, ground chia seeds, ground flaxseed, or sweet rice flour, or any mixture of two or more thereof).

The egg replacer according to any of the above can comprise between 0.01% and 5% by mass of a gum (e.g., any of the gums disclosed herein, such as, for example, arrowroot flour, xanthan gum, tara gum, guar gum, agar gum, locust bean gum, or gum arabic, or any mixture of two or more thereof), and between 0.1% and 20% by mass of an edible fiber (e.g., any of the edible fibers disclosed herein, such as, for example, psyllium husk, acacia fiber, citrus fiber, or cellulose, or any mixture of two or more thereof).

The egg replacer according to any of the above can comprise between 0.01% and 5% by mass of a gum (e.g., any of the gums disclosed herein, such as, for example, arrowroot flour, xanthan gum, tara gum, guar gum, agar gum, locust bean gum, or gum arabic, or any mixture of two or more thereof), and between 0.1% and 30% by mass of a flour (e.g., any of the flours disclosed herein, such as, for example, ground chia seeds, ground flaxseed, or sweet rice flour, or any mixture of two or more thereof).

The egg replacer according to any of the above can comprise between 0.1% and 20% by mass of an edible fiber (e.g., any of the edible fibers disclosed herein, such as, for example, psyllium husk, acacia fiber, citrus fiber, or cellulose, or any mixture of two or more thereof), and between 0.1% and 30% by mass of a flour (e.g., any of the flours disclosed herein, such as, for example, ground chia seeds, ground flaxseed, or sweet rice flour, or any mixture of two or more thereof).

The egg replacer according to any of the above can comprise between 0.05% and 40% by mass of a starch (e.g., any of the starches disclosed herein, such as, for example, tapioca starch, corn (maize) starch, potato starch, arrowroot starch, tapioca syrup solids, or rice syrup solids, or any mixture of two or more thereof), and between 0.01% and 5% by mass of a gum (e.g., any of the gums disclosed herein, such as, for example, arrowroot flour, xanthan gum, tara gum, guar gum, agar gum, locust bean gum, or gum arabic, or any mixture of two or more thereof), and between 0.1% and 20% by mass of an edible fiber (e.g., any of the edible fibers disclosed herein, such as, for example, psyllium husk, acacia fiber, citrus fiber, or cellulose, or any mixture of two or more thereof).

The egg replacer according to any of the above can comprise between 0.05% and 40% by mass of a starch (e.g., any of the starches disclosed herein, such as, for example, tapioca starch, corn (maize) starch, potato starch, arrowroot starch, tapioca syrup solids, or rice syrup solids, or any mixture of two or more thereof), and between 0.01% and 5% by mass of a gum (e.g., any of the gums disclosed herein, such as, for example, arrowroot flour, xanthan gum, tara gum, guar gum, agar gum, locust bean gum, or gum arabic, or any mixture of two or more thereof), and between 0.1% and 30% by mass of a flour (e.g., any of the flours disclosed herein, such as, for example, ground chia seeds, ground flaxseed, or sweet rice flour, or any mixture of two or more thereof).

The egg replacer according to any of the above can comprise between 0.05% and 40% by mass of a starch (e.g., any of the starches disclosed herein, such as, for example, tapioca starch, corn (maize) starch, potato starch, arrowroot starch, tapioca syrup solids, or rice syrup solids, or any mixture of two or more thereof), and between 0.1% and 20% by mass of an edible fiber (e.g., any of the edible fibers disclosed herein, such as, for example, psyllium husk, acacia fiber, citrus fiber, or cellulose, or any mixture of two or more thereof), and between 0.1% and 30% by mass of a flour (e.g., any of the flours disclosed herein, such as, for example, ground chia seeds, ground flaxseed, or sweet rice flour, or any mixture of two or more thereof).

The egg replacer according to any of the above can comprise between 0.01% and 5% by mass of a gum (e.g., any of the gums disclosed herein, such as, for example, arrowroot flour, xanthan gum, tara gum, guar gum, agar gum, locust bean gum, or gum arabic, or any mixture of two or more thereof), and between 0.1% and 20% by mass of an edible fiber (e.g., any of the edible fibers disclosed herein, such as, for example, psyllium husk, acacia fiber, citrus fiber, or cellulose, or any mixture of two or more thereof), and between 0.1% and 30% by mass of a flour (e.g., any of the flours disclosed herein, such as, for example, ground chia seeds, ground flaxseed, or sweet rice flour, or any mixture of two or more thereof).

The egg replacer according to any of the above can comprise between 0.05% and 40% by mass of a starch (e.g., any of the starches disclosed herein, such as, for example, tapioca starch, corn (maize) starch, potato starch, arrowroot starch, tapioca syrup solids, or rice syrup solids, or any mixture of two or more thereof), and between 0.01% and 5% by mass of a gum (e.g., any of the gums disclosed herein, such as, for example, arrowroot flour, xanthan gum, tara gum, guar gum, agar gum, locust bean gum, or gum arabic, or any mixture of two or more thereof), and between 0.1% and 20% by mass of an edible fiber (e.g., any of the edible fibers disclosed herein, such as, for example, psyllium husk, acacia fiber, citrus fiber, or cellulose, or any mixture of two or more thereof), and between 0.1% and 30% by mass of a flour (e.g., any of the flours disclosed herein, such as, for example, ground chia seeds, ground flaxseed, or sweet rice flour, or any mixture of two or more thereof).

The egg replacer according to any of the above can comprise such amount of a milk protein component consisting of one or more milk proteins selected from the group consisting of β-lactoglobulin, α-lactalbumin, lactoferrin, transferrin, serum albumin, lactoperoxidase, GMP, β-casein, γ-casein, κ-casein, α-S1-casein, α-S2-casein, and can further comprise such amount of one or more carbohydrates selected from the group consisting of a starch (e.g., any of the starches disclosed herein, and any mixture of two or more thereof), a gum (e.g., any of the gums disclosed herein, and any mixture of two or more thereof), an edible fiber (e.g., any of the edible fibers disclosed herein, and any mixture of two or more thereof), and a flour (e.g., any of the flours disclosed herein, and any mixture of two or more thereof), that the egg replacer has an egg attribute (e.g., any of the egg attributes disclosed above, and any combination of two or more thereof) that is between 50% and 150%, 140%, 130%, 120%, 110%, 100%, 90%, 80%, 70%, or 60%; between 60% and 150%, 140%, 130%, 120%, 110%, 100%, 90%, 80%, or 70%; between 70% and 150%, 140%, 130%, 120%, 110%, 100%, 90%, or 80%; between 80% and 150%, 140%, 130%, 120%, 110%, 100%, or 90%; between 90% and 150%, 140%, 130%, 120%, 110%, or 100%; between 100% and 150%, 140%, 130%, 120%, or 110%; between 110% and 150%, 140%, 130%, or 120%; between 120% and 150%, 140%, or 130%; between 130% and 150%, or 140%; or between 140% and 150% of such egg attribute of egg, or that is at least 1.5-fold, at least 2-fold, at least 3-fold, at least 4-fold, at least 5-fold, at least 6-fold, at least 7-fold, at least 8-fold, at least 9-fold, at least 10-fold, at least 15-fold, or at least 20-fold of such egg attribute of egg.

The food product can comprise such amount of an egg replacer according to any of the above that the food product has an egg attribute (e.g., any of the egg attributes disclosed herein, and any combination of two or more thereof) that is between 50% and 150%, 140%, 130%, 120%, 110%, 100%, 90%, 80%, 70%, or 60%; between 60% and 150%, 140%, 130%, 120%, 110%, 100%, 90%, 80%, or 70%; between 70% and 150%, 140%, 130%, 120%, 110%, 100%, 90%, or 80%; between 80% and 150%, 140%, 130%, 120%, 110%, 100%, or 90%; between 90% and 150%, 140%, 130%, 120%, 110%, or 100%; between 100% and 150%, 140%, 130%, 120%, or 110%; between 110% and 150%, 140%, 130%, or 120%; between 120% and 150%, 140%, or 130%; between 130% and 150%, or 140%; or between 140% and 150% of such egg attribute of a comparable food product, or of at least 1.5-fold, at least 2-fold, at least 3-fold, at least 4-fold, at least 5-fold, at least 6-fold, at least 7-fold, at least 8-fold, at least 9-fold, at least 10-fold, at least 15-fold, or at least 20-fold of such egg attribute of a comparable food product.

### Other Ingredients

The egg replacer according to any of the above can further comprise an other ingredient (e.g., any of the other ingredients disclosed herein), for example at between 0.001% and 50%, 25%, 20%, 15%, 10%, 9%, 8%, 7%, 6%, 5%, 4%, 3%, 2%, 1%, 0.5%, 0.1%, or 0.01%; between 0.01% and 50%, 25%, 20%, 15%, 10%, 9%, 8%, 7%, 6%, 5%, 4%, 3%, 2%, 1%, 0.5%, or 0.1%; between 0.1% and 50%, 25%, 20%, 15%, 10%, 9%, 8%, 7%, 6%, 5%, 4%, 3%, 2%, 1%, or 0.5%; between 0.5% and 50%, 25%, 20%, 15%, 10%, 9%, 8%, 7%, 6%, 5%, 4%, 3%, 2%, or 1%; between 1% and 50%, 25%, 20%, 15%, 10%, 9%, 8%, 7%, 6%, 5%, 4%, 3%, or 2%; between 2% and 50%, 25%, 20%, 15%, 10%, 9%, 8%, 7%, 6%, 5%, 4%, or 3%; between 3% and 50%, 25%, 20%, 15%, 10%, 9%, 8%, 7%, 6%, 5%, or 4%; between 4% and 50%, 25%, 20%, 15%, 10%, 9%, 8%, 7%, 6%, or 5%; between 5% and 50%, 25%, 20%, 15%, 10%, 9%, 8%, 7%, or 6%; between 6% and 50%, 25%, 20%, 15%, 10%, 9%, 8%, or 7%; between 7% and 50%, 25%, 20%, 15%, 10%, 9%, or 8%; between 8% and 50%, 25%, 20%, 15%, 10%, or 9%; between 9% and 50%, 25%, 20%, 15%, or 10%; between 10% and 50%, 25%, 20%, or 15%; between 15% and 50%, 25%, or 20%; between 20% and 50% or 25%; or between 25% and 50% by mass of any one such other ingredient, any combination of two or more such other ingredients, or all such other ingredients.

The egg replacer according to any of the above can comprise a nutritional agent (such as for example, a nutraceutical, nutritional supplement, prebiotic, probiotic, pro-vitamin, vitamin (e.g., vitamin A, vitamin B, vitamin E, vitamin D, vitamin K), mineral (e.g., calcium, phosphorous, potassium, sodium, citrate, chloride, phosphate, sulfate, magnesium, potassium, zinc, iron, molybdenum, manganese, copper), antioxidant, lipid, or non-milk protein) or a functional agent (such as, for example, an acidulant, buffering agent, shelf life extending agent, pH and/or ionic strength adjusting agent, anti-microbial agent, anti-oxidant, preservative, emulsifier, texturing/mouthfeel agent, coloring agent, taste/flavor agent, aroma agent, leavening agent, flow agent, bakery conditioners, or sweetening agent).

The egg replacer according to any of the above can comprise a lipid, such as, for example, a fat, oil, monoglyceride, diglyceride, triglyceride, phospholipid, free fatty acid, or mixture thereof.

The egg replacer according to any of the above can comprise an oil, such as, for example, a plant oil (e.g., sunflower oil, coconut oil, mustard oil, peanut oil, canola oil, corn oil, cottonseed oil, flax seed oil, olive oil, palm oil, rapeseed oil, safflower oil, sesame oil, soybean oil, almond oil, beech nut oil, brazil nut oil, cashew oil, hazelnut oil, macadamia nut oil, mongongo nut oil, pecan oil, pine nut oil, pistachio nut oil, walnut oil, avocado oil, grape oil), microbe-derived oil, algae-derived oil, fungus-derived oil, marine animal oil (e.g., Atlantic fish oil, Pacific fish oil, Mediterranean fish oil, light pressed fish oil, alkaline treated fish oil, heat treated fish oil, light and heavy brown fish oil, bonito oil, pilchard oil, tuna oil, sea bass oil, halibut oil, spearfish oil, barracuda oil, cod oil, menhaden oil, sardine oil, anchovy oil, capelin oil, Atlantic cod oil, Atlantic herring oil, Atlantic mackerel oil, Atlantic menhaden oil, salmonid oil, shark oil, squid oil, cuttle fish oil, octopus oil, krill oil, seal oil, whale oil), non-essential oil, essential oil, natural oil, non-hydrogenated oil, partially hydrogenated oil, hydrogenated oil (e.g., hydrogenated coconut oil), crude oil, semi-refined (also called alkaline refined) oil, interesterified oil, refined oil, or derivative or mixture thereof.

The egg replacer according to any of the above can comprise a monoglyceride or diglyceride, such as, for example, a plant-derived monoglyceride or diglyceride (e.g., monoglyceride or diglyceride derived from sunflower, coconut, peanut, cottonseed, olive, palm, rapeseed, safflower, sesame seed, soybean, almond, beech nut, Brazil nut, cashew, hazelnut, macadamia nut, mongongo nut, pecan, pine nut, pistachio, walnut, and avocado), or monoglyceride or diglyceride comprising an acyl chain of any of the free fatty acids disclosed herein, or any mixture of two or more thereof.

The egg replacer according to any of the above can comprise a free fatty acid, such as, for example, a butyric acid; caproic acid; caprylic acid; capric acid; lauric acid; myristic acid; palmitic acid; stearic acid; arachidic acid; behenic acid; lignoceric acid; cerotic acid; myristoleic acid; palmitoleic acid; sapienic acid; oleic acid; elaidic acid; vaccenic acid; linoleic acid; linoelaidic acid; α-linolenic acid; arachidonic acid; eicosapentaenoic acid; erucic acid; docosahexaenoic acid; omega-fatty acid (e.g., arachidonic acid, omega-3-fatty acid, omega-6-fatty acid, omega-7-fatty acid, omega-9-fatty acid); fatty acid with even number of carbons of 4-16 carbons in length; monosaturated acid [particularly with 18 carbons]; fatty acid with low interfacial tension (e.g., less than 20, less than 15, less than 11, less than 9, less than 7, less than 5, less than 3, less than 2, less than 1, or less than 0.5 dynes/cm, from 0.1 to 20, from 1 to 15, from 2 to 9, from 3 to 9, from 4 to 9, from 5 to 9, from 2 to 7, from 0.1 to 5, from 0.3 to 2, or from 0.5 to 1 dynes/cm, 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, or 20.0); butyric (4:0) acid or caproic (6:0) acid that is esterified at sn-3; medium-chain fatty acid (8:0-14:0) as well as 16:0 that are esterified at positions sn-1 and sn-2; fatty acid in which stearic acid (18:0) is placed at position sn-1; fatty acid in which oleic acid (18:1) is placed at positions sn-1 and sn-3; fatty acid that have a range of carbon atoms (e.g., from 8 to 40, from 10 to 38, from 12 to 36, from 14 to 34, from 16 to 32, from 18 to 30, or from 20 to 28 carbon atoms); fatty acid that comprise at least one unsaturated bond (i.e., carbon-carbon double or triple bond; e.g., at least 2, at least 3, at least 4, at least 5, at least 6, at least 7, or at least 8 carbon-carbon double bonds and/or triple bonds); fatty acid with conjugated unsaturated bond (i.e., at least one pair of carbon-carbon double and/or triple bonds are bonded together, without a methylene (CH2) group between them [e.g., 4CH:CHi CH:CHi]); or derivative (e.g., ester [e.g., methyl and ethyl ester], salt [e.g., sodium and potassium salt], triglyceride derivative, diglycerides derivative, monoglyceride derivative) thereof; or any mixture of two or more thereof.

The egg replacer according to any of the above can comprise a phospholipid, such as, for example, a lecithin phospholipid (e.g., soy lecithin phospholipid, sunflower lecithin phospholipid, cotton lecithin phospholipid, rapeseed lecithin phospholipid, rice bran lecithin phospholipid, corn lecithin phospholipid, flour lecithin phospholipid), cardiolipin, ceramide phosphocholine, ceramide phosphoethanolamine, glycerophospholipid, phosphatidic acid, phosphatidylcholine, phosphatidylethanolamine, phosphatidylinositol, phosphosphingolipid, phosphatidylserine, or derivative thereof, or any mixture of two or more thereof.

The egg replacer according to any of the above can comprise a triglyceride, such as, for a tributyrin; short-chain triglyceride; short-chain triglyceride comprising three oleic acid; short-chain triglyceride comprising hexanoic acid; short-chain triglyceride comprising hexanoic acid and butyric acid; short-chain triglyceride comprising hexanoic acid and decanoic acid; and short-chain triglyceride comprising one butyric, one hexanoic, and one octanoic acid; or derivative thereof, or any mixture of two or more thereof.

The egg replacer according to any of the above can comprise a non-milk protein (including a recombinant non-milk protein having mammalian PTMs, non-mammalian PTMs, or a mixture thereof, and/or lacking a mammalian PTM, and/or lacking an epitope that can elicit an immune response in a human or animal) obtained from an animal (such as, for example, an insect (e.g., fly), mammal (e.g. cow, sheep, goat, rabbit, pig, human), or bird (e.g., chicken)) or plant (such as, for example, pea, potato, faba bean, soybean, and canola). Non-limiting examples of non-milk proteins include serum proteins (e.g., albumin), egg proteins (e.g., any of the egg proteins disclosed herein), structural proteins (e.g., collagen, tropoelastin, elastin), and plant proteins (e.g., protein concentrates or protein isolates obtained from pea, potato, faba, soybean, or canola).

The egg replacer according to any of the above can comprise an anti-oxidant, such as, for example, α-tocopherol (e.g., tocopherol comprised in bovine milk), low molecular weight thiol (e.g., low molecular weight thiol comprised in bovine milk), retinol (e.g., retinol comprised in bovine milk), carotenoid (e.g., carotenoid comprised in cow milk, α-carotene, β-carotene, γ-carotene, lutein, zeaxanthin, astaxanthin), vitamin E, Azadirachta indica extract, riboflavin, rosemary extract, phenolic diterpene (e.g., carnosol, carnosic acid) comprised in rosemary extract, sage extract, ascorbic acid (vitamin C) and its salts, lactic acid and its salts, grape residue silage, phenolic compound (e.g., ferulic acid) comprised in grape residue silage, soybean (Glycine max) extract, isoflavone or polyphenolic compound comprised in soybean extract, garlic (Allium sativum) extract, phenolic or flavonoid, or terpenoid compound comprised in garlic extract, fennel (Foeniculum vulgare Mill.) extract, chamomile (Matricaria recutita L.) extract, fatty acid (e.g., alpha-lipoic acid), brown algae (e.g., Ascophyllum nodosum, Fucus vesiculosus), essential oil of green pink pepper (GEO), essential oil of mature pink pepper (MEO), green tea extract, butylated hydroxyanisole (E320), butylated hydroxytoluene (E321), polyphenol (e.g., curcumin, curcuminoid, desmethoxycurcumin (hydroxycirmamoyl feruloylmethane), bis-desmethoxycurcumin), catechin (e.g., epigallocatechin gallate, epicatechin gallate, epigallocatechin, epicatechin, C catechin, catechin comprised in green tea extract), or derivative thereof, or any mixture of two or more thereof.

The egg replacer according to any of the above can comprise a emulsifier, such as, for example, an anionic emulsifier, non-ionic emulsifier, cationic emulsifier, amphoteric emulsifier, bioemulsifier, steric emulsifier, Pickering emulsifier, glycolipids (e.g., trehalose lipid, sophorolipid, rhamnolipid, mannosylerythriol lipids), oligopeptides (e.g., gramicidin S, polymyxin), lipopeptides (e.g., surfactin), phospholipid, fatty acid, neutral lipid, polymeric biosurfactant, amphipathic polysaccharide, lipopolysaccharide, proteins (e.g., pea protein, soy protein, chickpea protein, algae protein, yeast protein, potato protein, lentil protein), mannoprotein, sodium phosphate, calcium stearoyl lactylate, mono- and diacetyl tartaric acid esters of monoglyceride, phospholipid, sorbitan monostearate, magnesium stearate, sodium/potassium/calcium salts of fatty acid, calcium stearoyl di lactate, poly-glycerol ester, sorbitan fatty acid ester, acetic acid esters of monoglyceride, lactic acid esters of monoglyceride, citric acid esters of monoglyceride, polyglycerol esters of fatty acid, polyglycerol polyricinoleate, propane-1,2-diol esters of fatty acid, sugar ester, sucrose esters of fatty acid, monoglyceride, acetylated monoglyceride, lactylated monoglyceride, diglyceride, phosphate monoglyceride, diacetyl tartaric acid ester, sodium/calcium stearoyl-2-lactylate, ammonium phosphatide, polysorbate, polysorbate-80, carboxymethylcellulose (CMC), modulated cellulose, citric acid ester, liposan, emulsan, lecithins (e.g., plant-based lecithin, garbanzo lecithin, fava bean lecithin, soy lecithin, sunflower lecithin, canola lecithin), surfactants (e.g., sorbitan trioleate (Span 85), sorbitan tristearate (Span 65), sorbitan sesquioleate (Arlacel 83), glyceryl monostearate, sorbitan monooleate (Span 80), sorbitan monostearate (Span 60), sorbitan monopalmitate (Span 40), sorbitan monolaurate (Span 20), polyoxyethylene sorbitan tristearate (Tween 65), polyoxyethylene sorbitan trioleate (Tween 85), polyethylene glycol 400 monostearate, polysorbate 60 (Tween 60), polyoxyethylene monostearate, polysorbate 80 (Tween 80), polysorbate 40 (Tween 40), polysorbate 20 (Tween 20), PEG 20 tristearate, PEG 20 trioleate, PEG 20 monostearate, PEG 20 monooleate, PEG 20 monopalmitate, and PEG 20 monolaurate sorbitan), and derivatives and mixtures thereof.

The egg replacer according to any of the above can comprise a sweetening agent, such as, for example, stevia, aspartame, cyclamate, saccharin, sucralose, mogrosides, brazzein, curculin, erythritol, glycyrrhizin, inulin, isomalt, lacititol, mabinlin, malititol, mannitol, miraculin, monatin, monelin, osladin, pentadin, sorbitol, thaumatin, xylitol, acesulfame potassium, advantame, alitame, aspartame-acesulfame, sodium cyclamate, dulcin, glucin, neohesperidin dihyrdochalcone, neotame, P-4000, honey, sucrose, corn syrup solids, glucose, lactose, galactose, dextrose, fructose, maltose, isomaltulose, trehalose, maltodextrin, asulfame K, cyclamates, L-aspartyl-L-phenylalanine, tagatose, stevioside, hydrogenated starch hydrolysates, high-fructose corn syrup, fructooligosaccharides, polydextrose, or derivative or mixture thereof.

### Composition Comprising Egg Replacer

In another aspect, provided herein is a composition comprising the egg replacer according to any of the above, wherein the egg replacer imparts or materially contributes to an egg attribute of the composition.

The composition according to any of the above can comprise at least 0.001%, 0.01%, 0.1%, 1%, 10%, 20%, 30%, 40%, 50%, 60%, 70%, 80%, 90%, 95%, or 99%; between 0.001% and 100%, 99%, 95%, 90%, 80%, 70%, 60%, 50%, 40%, 30%, 20%, 10%, 1%, 0.1%, or 0.01%; between 0.01% and 100%, 99%, 95%, 90%, 80%, 70%, 60%, 50%, 40%, 30%, 20%, 10%, 1%, or 0.1%; between 0.1% and 100%, 99%, 95%, 90%, 80%, 70%, 60%, 50%, 40%, 30%, 20%, 10%, or 1%; between 1% and 100%, 99%, 95%, 90%, 80%, 70%, 60%, 50%, 40%, 30%, 20%, or 10%; between 10% and 100%, 99%, 95%, 90%, 80%, 70%, 60%, 50%, 40%, 30%, or 20%; between 20% and 100%, 99%, 95%, 90%, 80%, 70%, 60%, 50%, 40%, or 30%; between 30% and 100%, 99%, 95%, 90%, 80%, 70%, 60%, 50%, or 40%; between 40% and 100%, 99%, 95%, 90%, 80%, 70%, 60%, or 50%; between 50% and 100%, 99%, 95%, 90%, 80%, 70%, or 60%; between 60% and 100%, 99%, 95%, 90%, 80%, or 70%; between 70% and 100%, 99%, 95%, 90%, or 80%; between 80% and 100%, 99%, 95%, or 90%; between 90% and 100%, 99%, or 95%; between 95% and 100% or 99%; or between 99% and 100% by mass of the egg replacer.

The composition according to any of the above can be essentially free of a component obtained from an animal (e.g., any or all components obtained from an animal disclosed herein), or comprise 2% or less by mass of a component obtained from an animal.

The composition according to any of the above can be essentially free of one or more egg proteins (e.g., one or more of ovomucoid, ovalbumin, ovotransferrin, G162M F167A ovomucoid, ovoglobulin G2, ovoglobulin G3, α-ovomucin, β-ovomucin, lysozyme, ovoinhibitor, ovoglycoprotein, flavoprotein, ovomacroglobulin, ovostatin, cystatin, avidin, ovalbumin related protein X, and ovalbumin related protein Y).

The composition according to any of the above can have an allergenicity that is lower than that of a comparable composition, such as, for example, an allergenicity of up to 0%, 1%, 5%, 10%, 15%, 20%, 25%, 30%, 35%, 40%, 45%, 50%, 55%, 60%, 65%, 70%, 75%, 80%, 85%, 90%, or 95% relative to that of a comparable composition.

The composition according to any of the above upon heating can comprise a milk protein polymer network.

The composition according to any of the above can be a variety of products, including, for example, a food product (i.e., product that is ingested for dietary purposes), beverage product, pharmaceutical product, or personal care product.

Non-limiting examples of suitable food products include: emulsions, such as, for example, mayonnaise, dressings, soups, sauces, and dessert sauces (e.g., sabayon); fining agents, such as, for example, agents used for clarifying beverages, wines, and consommé; batters, such as, for example, cookie batters (e.g., batters used for production of any of the cookies disclosed herein), cake batters (e.g., batters used for production of any of the cakes disclosed herein), pastry batters (e.g., batters used for production of any of the pastries disclosed herein), crepe batters, waffle batters, donut batters, popover batters, and batters used for breading foods for frying (e.g., tempura, fritter); doughs, such as, for example, cake doughs (e.g., doughs used for production of any of the cakes disclosed herein), cookie doughs (e.g., doughs used for production of any of the cookies disclosed herein), bread doughs (e.g., doughs used for production of any of the breads disclosed herein), and pasta doughs; baked/cooked food products, such as, for example, cookies (e.g., biscotti, chocolate chip cookies, sugar cookies, waffle cookies, tuile, macaron, coconut macaroons), cakes (e.g., pound cake, yellow cake, devil's food cake, angel food cake, sponge cake, chiffon cake, tube cake, rolled cake, oil cake, castellas, pancakes, muffins, cupcakes, brownies, madeleines, cornbread, flourless cakes, molten chocolate cakes, financier, dacquoise, cheesecake, shortcake), pastries (e.g., danish, choux, pie crusts, puff pastry, croissants, crepes, tarts, scones, meringue, waffles, canelé, pate sucree, sable, souffles, marshmellows), and breads, (e.g., bagels, sweet buns, loaf breads, baguettes, pizza crusts, breadsticks, brioches, challahs, biscuits, hot dog rolls, hamburger buns, crispbreads, flatbreads, wraps, biscuits, gluten-free breads, pretzels, crackers, donuts, beignets, liege waffles); and egg-based food products, such as, for example, scrambled eggs, omelets, frittatas, pastas, custards (e.g., crème anglaise, pastry creams, pots de crème, crème caramel, crème brulee, bread puddings, french toasts, ice creams, curds (e.g., lemon curd)), sabayon, mousses, souffles, puddings, snacks, bars, quiches, meringues (e.g., French meringue, Swiss meringue, Italian meringue), meringue-based confections (e.g., marshmallows, nougat, macaron), meringue-based frostings (e.g., boiled icing, Swiss meringue buttercream, Italian meringue buttercream, French buttercream), and royal icing.

The food product according to any of the above can comprise no additional milk protein other than the milk protein comprised in the milk protein component comprised in the egg replacer.

The food product according to any of the above can be essentially free of a saturated lipid, or comprise less than 3%, less than 2%, less than 1%, less than 0.5%, or less than 0.1% by mass of a saturated lipid.

The food product according to any of the above can be essentially free of cholesterol, or comprise less than 1.5%, less than 1%, less than 0.5%, less than 0.1%, or less than 0.05% by mass of cholesterol.

The food product according to any of the above can be essentially free of an emulsifying agent (e.g., any of the emulsifying agents disclosed herein).

The food product according to any of the above can have an allergenicity that is lower than that of a comparable food product, such as, for example, an allergenicity of up to 0%, 1%, 5%, 10%, 15%, 20%, 25%, 30%, 35%, 40%, 45%, 50%, 55%, 60%, 65%, 70%, 75%, 80%, 85%, 90%, or 95% relative to that of a comparable food product.

The food product according to any of the above can be essentially free of one or more egg proteins (e.g., one or more egg proteins disclosed herein), gluten, and/or one or more native milk proteins.

### Egg Attribute

The egg replacer according to any of the above or the composition comprising the egg replacer according to any of the above has an egg attribute that is imparted or materially contributed to by the milk protein component comprised in the egg replacer or by the egg replacer comprised in the composition, respectively.

The egg attribute can be a physical attribute, mechanical attribute, chemical/biological attribute, sensory attribute, or functional attribute, or any combination thereof.

Variables that can be titrated to modulate such egg attribute include amount of the milk protein component comprised in the egg replacer, types and/or amounts of milk proteins comprised in the milk protein component, types and/or amounts of other ingredients (e.g., carbohydrates) comprised in the egg replacer, and methods of manufacture of the egg replacer.

### Physical Attribute

The egg replacer according to any of the above, or the composition comprising the egg replacer according to any of the above, can have a physical attribute that is a physical attribute of egg, or that is a physical attribute imparted on or materially contributed to a comparable composition by egg, respectively, such as, for example, an appearance (e.g., browning, color, translucence, opaqueness), a shape (e.g., rise/height, dome shape, spread; see Example 3), a structure (e.g., air cell average size, air cell size distribution, air cell wall thickness), a viscosity, a density, a gelling point/profile (i.e., temperature and time line at which gel formation sets in), and/or a melting point/profile.

Appearance can be qualitatively assessed (e.g., browning or color by a panel of human sensory experts), or quantitatively measured (e.g., color by spectroscopic analysis in L*a*b* color space). Rise/height and dome shape can be qualitatively assessed, or quantitatively measured, as described in Example 3. Spread can be qualitatively assessed, or quantitatively measured by weighing out a specific mass of a composition (e.g., a food product, a dough, a batter) and forming a mass or volume of specific dimensions (e.g., a ball of dough having a specific diameter and/or circumference, a cup of batter), placing the mass in the middle of a surface (e.g., baking sheet, griddle), and heating for a specific amount of time at a specific temperature), and comparing the (roughly circular) spread (e.g., radius, area) of the heated composition. Structure can be qualitatively assessed (e.g., by visual inspection) or quantitatively scored using microscopy. Viscosity and gelling point/profile can be qualitatively assessed (e.g., by rate or ease of flow, ease of movement during handling), or quantitatively measured using a viscometer or rheometer (e.g., using rotational viscometric methods, capillary viscometric methods, vibratory viscometric methods, ultrasonic pulse echo methods, pycnometric methods, or areometric methods). Density can be quantitatively measured by densymetric methods. Melting point can be quantitatively measured using calorimetry.

The composition comprising the egg replacer according to any of the above can have an appearance that is an appearance that is imparted on or materially contributed to a comparable composition by egg, such as, for example, a color (e.g., yellowish color) or browning that is similar to that imparted on or materially contributed to a comparable food product by egg.

Alternatively, the composition comprising the egg replacer according to any of the above can have an appearance that is different from an appearance that is imparted on or materially contributed to a comparable composition by egg, such as, for example, a color (e.g., whitish color) or browning that is different to that imparted on or materially contributed to a comparable food product by egg.

The egg replacer according to any of the above can have a physical attribute (e.g., any of the physical attributes disclosed above, including browning, color, spread, viscosity, density, and gelling point/profile), or a combination of two or more physical attributes (e.g., any combination of two or more of the physical attributes disclosed above), that is between 50% and 150%, 140%, 130%, 120%, 110%, 100%, 90%, 80%, 70%, or 60%; between 60% and 150%, 140%, 130%, 120%, 110%, 100%, 90%, 80%, or 70%; between 70% and 150%, 140%, 130%, 120%, 110%, 100%, 90%, or 80%; between 80% and 150%, 140%, 130%, 120%, 110%, 100%, or 90%; between 90% and 150%, 140%, 130%, 120%, 110%, or 100%; between 100% and 150%, 140%, 130%, 120%, or 110%; between 110% and 150%, 140%, 130%, or 120%; between 120% and 150%, 140%, or 130%; between 130% and 150%, or 140%; or between 140% and 150% of such physical attribute of egg, or that is at least 1.5-fold, at least 2-fold, at least 3-fold, at least 4-fold, at least 5-fold, at least 6-fold, at least 7-fold, at least 8-fold, at least 9-fold, at least 10-fold, at least 15-fold, or at least 20-fold of such physical attribute of egg.

The batter or dough comprising the egg replacer according to any of the above can have a physical attribute (e.g., any of the physical attributes disclosed above, including color, spread, and viscosity), or a combination of two or more physical attributes (e.g., any combination of two or more of the physical attributes disclosed above), that is between 50% and 150%, 140%, 130%, 120%, 110%, 100%, 90%, 80%, 70%, or 60%; between 60% and 150%, 140%, 130%, 120%, 110%, 100%, 90%, 80%, or 70%; between 70% and 150%, 140%, 130%, 120%, 110%, 100%, 90%, or 80%; between 80% and 150%, 140%, 130%, 120%, 110%, 100%, or 90%; between 90% and 150%, 140%, 130%, 120%, 110%, or 100%; between 100% and 150%, 140%, 130%, 120%, or 110%; between 110% and 150%, 140%, 130%, or 120%; between 120% and 150%, 140%, or 130%; between 130% and 150%, or 140%; or between 140% and 150% of such physical attribute of a comparable batter or dough, or that is at least 1.5-fold, at least 2-fold, at least 3-fold, at least 4-fold, at least 5-fold, at least 6-fold, at least 7-fold, at least 8-fold, at least 9-fold, at least 10-fold, at least 15-fold, or at least 20-fold of such physical attribute of a comparable batter or dough.

The baked/cooked food product comprising the egg replacer according to any of the above can have a physical attribute (e.g., any of the physical attributes disclosed above, including browning, color, rise/height, dome shape, and density), or a combination of two or more physical attributes (e.g., any combination of two or more of the physical attributes disclosed above), that is between 50% and 150%, 140%, 130%, 120%, 110%, 100%, 90%, 80%, 70%, or 60%; between 60% and 150%, 140%, 130%, 120%, 110%, 100%, 90%, 80%, or 70%; between 70% and 150%, 140%, 130%, 120%, 110%, 100%, 90%, or 80%; between 80% and 150%, 140%, 130%, 120%, 110%, 100%, or 90%; between 90% and 150%, 140%, 130%, 120%, 110%, or 100%; between 100% and 150%, 140%, 130%, 120%, or 110%; between 110% and 150%, 140%, 130%, or 120%; between 120% and 150%, 140%, or 130%; between 130% and 150%, or 140%; or between 140% and 150% of such physical attribute of a comparable baked/cooked food product, or that is at least 1.5-fold, at least 2-fold, at least 3-fold, at least 4-fold, at least 5-fold, at least 6-fold, at least 7-fold, at least 8-fold, at least 9-fold, at least 10-fold, at least 15-fold, or at least 20-fold of such physical attribute of a comparable baked/cooked food product.

### Mechanical Attribute

The egg replacer according to any of the above, or the composition comprising the egg replacer according to any of the above, can have a mechanical attribute that is a mechanical attribute of egg, or that is imparted on or materially contributed to a comparable composition by egg, respectively, such as, for example, a hardness/firmness, an adhesiveness, a resilience/recoverable energy, a structural integrity/cohesiveness, an elasticity/springiness/rebound, or a chewiness/breakdown.

Mechanical attributes can be quantitatively measuring using, for example, a texture analyzer (see Example 3).

The egg replacer according to any of the above can have a mechanical attribute or a combination of two or more mechanical attributes that is between 50% and 150%, 140%, 130%, 120%, 110%, 100%, 90%, 80%, 70%, or 60%; between 60% and 150%, 140%, 130%, 120%, 110%, 100%, 90%, 80%, or 70%; between 70% and 150%, 140%, 130%, 120%, 110%, 100%, 90%, or 80%; between 80% and 150%, 140%, 130%, 120%, 110%, 100%, or 90%; between 90% and 150%, 140%, 130%, 120%, 110%, or 100%; between 100% and 150%, 140%, 130%, 120%, or 110%; between 110% and 150%, 140%, 130%, or 120%; between 120% and 150%, 140%, or 130%; between 130% and 150%, or 140%; or between 140% and 150% of such mechanical attribute of egg, or that is at least 1.5-fold, at least 2-fold, at least 3-fold, at least 4-fold, at least 5-fold, at least 6-fold, at least 7-fold, at least 8-fold, at least 9-fold, at least 10-fold, at least 15-fold, or at least 20-fold of such mechanical attribute of egg.

The batter or dough comprising the egg replacer according to any of the above can have a mechanical attribute or a combination of two or more mechanical attributes that is between 50% and 150%, 140%, 130%, 120%, 110%, 100%, 90%, 80%, 70%, or 60%; between 60% and 150%, 140%, 130%, 120%, 110%, 100%, 90%, 80%, or 70%; between 70% and 150%, 140%, 130%, 120%, 110%, 100%, 90%, or 80%; between 80% and 150%, 140%, 130%, 120%, 110%, 100%, or 90%; between 90% and 150%, 140%, 130%, 120%, 110%, or 100%; between 100% and 150%, 140%, 130%, 120%, or 110%; between 110% and 150%, 140%, 130%, or 120%; between 120% and 150%, 140%, or 130%; between 130% and 150%, or 140%; or between 140% and 150% of such mechanical attribute of a comparable batter or dough, or that is at least 1.5-fold, at least 2-fold, at least 3-fold, at least 4-fold, at least 5-fold, at least 6-fold, at least 7-fold, at least 8-fold, at least 9-fold, at least 10-fold, at least 15-fold, or at least 20-fold of such mechanical attribute of a comparable batter or dough.

The baked/cooked food product comprising the egg replacer according to any of the above can have a mechanical attribute (e.g., any of the mechanical attributes disclosed above, including hardness/firmness, adhesiveness, resilience/recoverable energy, structural integrity/cohesiveness, elasticity/springiness/rebound, and chewiness/breakdown), or a combination of two or more mechanical attributes (e.g., any combination of two or more of the mechanical attributes disclosed above), that is between 50% and 150%, 140%, 130%, 120%, 110%, 100%, 90%, 80%, 70%, or 60%; between 60% and 150%, 140%, 130%, 120%, 110%, 100%, 90%, 80%, or 70%; between 70% and 150%, 140%, 130%, 120%, 110%, 100%, 90%, or 80%; between 80% and 150%, 140%, 130%, 120%, 110%, 100%, or 90%; between 90% and 150%, 140%, 130%, 120%, 110%, or 100%; between 100% and 150%, 140%, 130%, 120%, or 110%; between 110% and 150%, 140%, 130%, or 120%; between 120% and 150%, 140%, or 130%; between 130% and 150%, or 140%; or between 140% and 150% of such mechanical attribute of a comparable baked/cooked food product, or that is at least 1.5-fold, at least 2-fold, at least 3-fold, at least 4-fold, at least 5-fold, at least 6-fold, at least 7-fold, at least 8-fold, at least 9-fold, at least 10-fold, at least 15-fold, or at least 20-fold of such mechanical attribute of a comparable baked/cooked food product.

Chemical/Biological Attribute

The egg replacer according to any of the above, or the composition comprising the egg replacer according to any of the above, can have a chemical/biological attribute that is a chemical/biological attribute of egg, or that is imparted on or materially contributed to a comparable composition by egg, respectively, such as, for example, a nutrient content (e.g., types and/or amounts of proteins, types and/or amounts of amino acids, PDCAAS, BV), a pH, a digestibility, an absorption (e.g., proportion of absorbed protein from a food product), an oxidation stability, or a satiety regulation.

Nutrient content can be assessed using analytical methods known in the art, including but not limited to AOAC International reference methods AOAC 990.03 and AOAC 992.15, electrophoresis (e.g., SDS-PAGE), liquid column chromatography, immunochemical tests, or on-chip electrophoresis (e.g., using the Agilent Protein 80 kit and the Agilent 2100 Bioanalyzer) for determination of type and/or content of proteins and amino acids. Alternatively, chemical/biological attributes can be calculated from the nutrient contents of ingredients.

The egg replacer according to any of the above can comprise between 5% and 100%, 90%, 80%, 70%, 60%, 50%, 40%, 30%, 20%, 10%; between 10% and 100%, 90%, 80%, 70%, 60%, 50%, 40%, 30%, or 20%; between 20% and 100%, 90%, 80%, 70%, 60%, 50%, 40%, or 30%; between 30% and 100%, 90%, 80%, 70%, 60%, 50%, or 40%; between 40% and 100%, 90%, 80%, 70%, 60%, or 50%; between 50% and 100%, 90%, 80%, 70%, or 60%; between 60% and 100%, 90%, 80%, or 70%; between 70% and 100%, 90%, or 80%; between 80% and 100%, or 90%; or between 90% and 100% by mass of protein.

The egg replacer according to any of the above can have a PDCAAS of at least 0.8, at least 0.82, at least 0.84, at least 0.86, at least 0.88, at least 0.9, at least 0.92, at least 0.94, at least 0.96, or at least 0.98.

The egg replacer according to any of the above can comprise between 5% and 30%, 25%, 20%, 15%, or 10%; between 10% and 30%, 25%, 20%, or 15%; between 15% and 30%, 25%, or 20%; between 20% and 30%, or 25%; or between 25% and 30% by dry mass of branched-chain amino acids.

The egg replacer according to any of the above can have a BV of at least 80, at least 85, at least 90, at least 95, at least 100, at least 105, at least 110, at least 115, or at least 120.

### Functional Attribute

The egg replacer according to any of the above, or the composition comprising the egg replacer according to any of the above, can have a functional attribute that is a functional attribute of egg, or that is imparted on or materially contributed to a comparable composition by egg, respectively, such as, for example, a foaming/leavening behavior (e.g., foaming capacity (i.e., capacity to form a foam, wherein the term “foam” as used herein refers to air bubbles dispersed in a solid or aqueous continuous phase), foam yield stress (i.e., stress required to initiate flow in the foam), foam stability (i.e., half-life of foam in response to a physical and/or chemical condition), foam drainage (i.e., rate at which a foam destabilizes and an aqueous phase begins to drain from the foam)), a gelling/thickening/coagulating behavior (e.g., gelling capacity (i.e., capacity to form a gel, wherein the term “gel” as used herein refers to a protein network with spaces filled with solvent linked by hydrogen bonds to the protein molecules), gelling profile (e.g., curve of gelling capacity over time, viscoelastic parameters as a function of temperature), gel strength (i.e., mechanical force required to break a gel surface of a defined area)), an emulsifying behavior (e.g., emulsifying capacity (i.e., capacity to stabilize an emulsion or the amount of oil that a given mass of sample can emulsify without destabilization), emulsion stability (i.e., half-life of an emulsion produced under given conditions, such as, for example, a given protein concentration, lipid concentration, pH, ionic strength, or preparation method)), a water binding behavior (e.g., water binding capacity (i.e., capacity to bind water), water binding strength), syneresis upon gelling (i.e., water weeping over time), and a use versatility (i.e., potential for varied use and production of a diversity of compositions).

Foaming/leavening behavior can be assessed using methods know in the art, including, for example, measurement of the percentage of air incorporated in a foam formed by a composition after whipping at a specified speed and for a specified amount of time under defined conditions (e.g., temperature, pH, ionic strength, protein concentration, carbohydrate concentration) (see Example 2), measurement of how long it takes for a given mass of foam to destabilize in the form of liquid draining or seeping, measurement of yield stress under shear or the amount of stress required to initiate flow in a sample, and measurement of the height and degree of airy, light texture of a baked/cooked food product produced using the egg replacer provided herein or egg.

Gelling/thickening/coagulating behavior can be assessed using methods known in the art, such as, for example, measurement of the time required for a composition to form a gel under defined conditions (e.g., temperature, pH, ionic strength, protein concentration, carbohydrate concentration), measurement of storage and elastic moduli and phase angle obtained in frequency sweeps on a rheometer, and measurement of resistance of a gel to a physical force and/or chemical condition (e.g., agitation, temperature, pH, ionic strength, protein concentration, sugar concentration, ionic strength) (see, for example, Ikeda et al. 2000. Biopolymers, 56: 109-119).

Emulsifying behavior can be assessed using methods know in the art, including, for example, preparation of a lipid in water emulsion under defined conditions (e.g., mixing apparatus, mixing speed, mixing time) and subsequent measurement of stability over time of phase separation in a mixture of lipid and water, measuring the rate of creaming or sedimentation, measuring the change in opacity over time, or measuring the change of dispersed phase particle size over time (see, for example, Liang et al. 2013. Food Hydrocolloids, 33, 297-308).

Water binding behavior can be assessed using methods know in the art, including, for example, measurement of the amount of water exuded by a sample after centrifugation, which is used to determine the amount of water bound per mass of protein, or development of moisture sorption isotherms based on mass of water bound per mass of protein as a function of vapor pressure.

The egg replacer according to any of the above can have a functional attribute (e.g., any of the functional attributes disclosed above, including foaming capacity, foam yield stress, foam stability, foam drainage, gelling capacity, gel strength, emulsifying capacity, emulsion stability, water binding capacity, and syneresis upon gelling), or a combination of two or more functional attributes (e.g., any combination of two or more of the functional attributes disclosed above), that is between 50% and 150%, 140%, 130%, 120%, 110%, 100%, 90%, 80%, 70%, or 60%; between 60% and 150%, 140%, 130%, 120%, 110%, 100%, 90%, 80%, or 70%; between 70% and 150%, 140%, 130%, 120%, 110%, 100%, 90%, or 80%; between 80% and 150%, 140%, 130%, 120%, 110%, 100%, or 90%; between 90% and 150%, 140%, 130%, 120%, 110%, or 100%; between 100% and 150%, 140%, 130%, 120%, or 110%; between 110% and 150%, 140%, 130%, or 120%; between 120% and 150%, 140%, or 130%; between 130% and 150%, or 140%; or between 140% and 150% of such functional attribute of egg, or that is at least 1.5-fold, at least 2-fold, at least 3-fold, at least 4-fold, at least 5-fold, at least 6-fold, at least 7-fold, at least 8-fold, at least 9-fold, at least 10-fold, at least 15-fold, or at least 20-fold of such mechanical attribute of egg.

The egg replacer according to any of the above can have a use versatility (i.e., ability to use the egg replacer in a variety of manners and/or to derive a diversity of other compositions from the egg replacer) that is similar to that of egg. A non-limiting example of such use versatility is production of any of the compositions provided herein that comprise the egg replacer (i.e., batters, doughs, baked/cooked food products, emulsions, egg-based food products).

### Sensory Attribute

The egg replacer according to any of the above, or the composition comprising the egg replacer according to any of the above, can have a sensory attribute that is a sensory attribute of egg, or that is imparted on or materially contributed to a comparable composition by egg, respectively, such as, for example, a taste/flavor, an aroma, or an eating quality (e.g., fattiness, creaminess, richness, greasiness, thickness, hardness/firmness, crispiness, crumbliness, crunchiness, chewiness, chewdown, tenderness, compactness, cohesiveness, adhesiveness, graininess, smoothness, juiciness, wetness, mouthcoating, slipperiness on tongue, roughness, abrasiveness, uniformity of bite and/or chew, springiness, texture, airiness, effort required to draw sample from spoon over tongue).

Sensory attributes can be qualitatively assessed by a panel of sensory experts, or derived from correlated physical, mechanical, chemical, and/or functional attributes that impact textural and sensory perceptions (e.g., hardness/firmness, adhesiveness, resilience/recoverable energy, structural integrity/cohesiveness, elasticity/springiness/rebound, chewiness/breakdown, density, viscosity, content/size/shape of solid particles, water binding).

The food product comprising the egg replacer according to any of the above can have a sensory attribute (e.g., taste/flavor, eating quality) that is similar to that imparted on or materially contributed to a comparable food product by egg.

Alternatively, the food product comprising the egg replacer according to any of the above can have a sensory attribute (e.g., taste/flavor, eating quality) that is different to that imparted on or materially contributed to a comparable food product by egg, such as, for example, a non-eggy taste/flavor or a neutral taste/flavor (i.e., not affecting the taste/flavor of the food product).

### Method for Producing Egg Replacer

In another aspect, provided herein is a method for producing the egg replacer provided herein. The method comprises the step of obtaining one or more native and/or recombinant milk proteins.

A recombinant milk protein can be obtained by culturing a suitable recombinant host cell comprising a recombinant polynucleotide (e.g., recombinant vector) encoding the recombinant milk protein in a culture medium under conditions suitable for production and/or secretion of the recombinant milk protein. The method can further comprise the steps of: a) obtaining a recombinant polynucleotide encoding the milk protein, and introducing the recombinant polynucleotide into host cell to obtain the recombinant host cell; and/or b) isolating (i.e., purifying) the recombinant milk protein; and/or c) post-processing the recombinant milk protein.

Alternatively, a recombinant milk protein can be obtained using in vitro methods (e.g., using cell-free transcription and/or translation systems).

A native milk protein can be isolated from milk. Methods for isolating protein from natural sources are known and used to produce protein isolates, protein concentrates, protein flours, and partially purified or purified proteins.

### Recombinant Polynucleotide

The recombinant polynucleotide comprised in the recombinant host cell can be prepared by any suitable method known in the art, including, without limitation, direct chemical synthesis and cloning.

The recombinant polynucleotide typically comprises one or more expression cassettes, wherein each expression cassette comprises: a promoter (e.g., a fungal promoter), an optional signal sequence (i.e., a sequence that encodes a peptide that mediates the delivery of a nascent protein attached to the peptide to the exterior of the cell in which the nascent protein is synthesized (i.e., a sequence that encodes a peptide that mediates secretion of the recombinant protein)), a sequence encoding the milk protein, and a termination sequence (or multiple termination sequences), wherein the promoter is operably linked in sense orientation to the optional signal sequence (i.e., the promoter and optional signal sequence and subsequent sequence encoding the milk protein are positioned such that the promoter is effective for regulating transcription of the optional signal sequence and sequence encoding the milk protein), the optional signal sequence is operably linked in sense orientation to the sequence encoding the milk protein (i.e., the signal sequence and sequence encoding the milk protein are positioned such that transcription and translation produces the milk protein comprising a functional signal sequence), and the termination sequence is operably linked to the sequence encoding the milk protein (i.e., the sequence encoding the milk protein and the termination sequence are positioned such that the terminator is effective for terminating transcription of the optional signal sequence and sequence encoding the milk protein).

The promoter may be any suitable promoter that is functional in the recombinant host cell. In some embodiments, the promoter is a constitutive promoter. In other embodiments, the promoter is an inducible promoter or a repressible promoter (e.g., a promoter that is induced or repressed in the presence of glucose, galactose, lactose, sucrose, cellulose, sophorose, gentiobiose, sorbose, disaccharides that induce the cellulase promoters, starch, tryptophan, or phosphate).

Non-limiting examples of suitable promoters for use in recombinant fungal host cells include promoters, and functional parts thereof, of genes encoding any of the following proteins: glucoamylase (e.g., glaA of Aspergillus niger, Aspergillus awamori, Aspergillus japonicus, Aspergillus tubingensis, Aspergillus foetidus, or Aspegillus carbonarius), amylase (e.g., Aspergillus oryzae TAKA amylase, Aspergillus niger neutral alpha-amylase, Aspergillus niger acid stable alpha-amylase, fungal α-amylase [amy], bacterial alpha-amylase), protease (e.g., Rhizomucor miehei aspartic protease, Aspergillus oryzae alkaline protease, Fusarium oxysporum trypsin-like protease, Trichoderma reesei protease), lipase (e.g., Rhizomucor miehei lipase), isomerase (e.g., Aspergillus oryzae triose phosphate isomerase, fungal triose phosphate isomerase [tpi], yeast triosephosphate isomerase), acetamidase (e.g., Aspergillus nidulans or Aspergillus oryzae or other fungal acetamidase [amdS]), dehydrogenase (e.g., fungal alcohol dehydrogenase [adhA], fungal glyceraldehyde-3-phosphate dehydrogenase [gpd], yeast alcohol dehydrogenase), xylanase (e.g., fungal xylanase [xlnA], Trichoderma xylanases [xyn1, xyn2, bxl1]), kinase (e.g., yeast 3-phosphoglycerate kinase), hydrolase (e.g., fungal cellobiohydrolase I [cbh1], Trichoderma hydrolases [cbh2, egl1, egl2]), phosphatase (e.g., Fusarium acid phosphatase), and other fungal proteins (e.g., fungal endo α-L-arabinase [abnA], fungal α-L-arabinofuranosidase A [abfA], fungal α-L-arabinofuranosidase B [abfB], fungal phytase, fungal ATP-synthetase, fungal subunit 9 [oliC], fungal sporulation-specific protein [Spo2], fungal SSO, yeast alcohol oxidase, yeast lactase, Neurospora crassa CPC1, Aspergillus nidulans trpC, fungal chitinolytic enzymes [e.g., endo- & exo-chitinase, beta-glucanase], fungal VAMP-associated proteins [VAPs], fungal translation elongation factor [TEF1], fungal DNA damage-responsive protein [DDRP], fungal [e.g., Fusarium or Neurospora crassa] hexagonal peroxisome [Hex1], fungal [e.g., Neurospora crassa] catalase), and any other protein produced at high level in the recombinant fungal host cell.

Non-limiting examples of suitable promoters for use in recombinant bacterial or yeast host cells include promoters, and functional parts thereof, of genes encoding any of the following proteins: LAC4, T7 polymerase, TAC, GAL1, λPL, λPR, beta-lactamase, spa, CYC1, TDH3, GPD, TEF1, ENO2, PGL1, GAP, SUC2, ADH1, ADH2, HXT7, PHO5, CLB1, AOX1, cellulase, amylase, protease, xylanase, and any other protein produced at high level in the recombinant bacterial or yeast host cell, such as, for example, promoters of a stress (e.g., heat shock) response genes (e.g., hacl, BIP).

The signal sequence may be any suitable signal sequence that is functional in the recombinant host cell. Non-limiting examples of suitable signal sequences for use in recombinant fungal host cells include signal sequences from Trichoderma reesei (e.g., signal sequence of Cbh1, cbh2, egl1, egl2, xyn1, xyn2, bxl1, hfb1, or hfb2), signal sequences from Aspergillus niger (e.g., signal sequence of glaA, amyA, amyC, or aamA), signal sequences from yeast (e.g., signal sequence of alpha mating factor [MFa]), and functional parts thereof. Non-limiting examples of suitable signal sequences for use in recombinant yeast host cells include signal sequences from yeast (e.g., signal sequence of alpha mating factor [MFa]), and functional parts thereof.

The termination sequence may be any suitable termination sequence that is functional in the recombinant host cell. Non-limiting examples of suitable termination sequences for use in recombinant fungal host cells include but are not limited to termination sequences of Aspergillus oryzae (e.g., termination sequence of TAKA amylase gene), Aspergillus niger (e.g., termination sequence of glaA, gpdA, aamA, trpC, pdc1, adh1, amdS, or tef1 gene), Fusarium oxysporum (e.g., termination sequence of serine protease [trypsin] gene), Trichoderma reesei (e.g., termination sequence of cbh1, pdc1, TEF1, gpd1, xyn1, or adh1 gene), Pichia pastoris (e.g., termination sequence of aox1, gap1, adh1, tef1, tps1, or pgk1 gene), Saccharomyces cerevisiae (e.g., termination sequence of adh1, cyc1, gal1, tef1, pdc1, pgk1, or tps1 gene), synthetic termination sequences, and any combination of the above listed sequences. Non-limiting examples of suitable termination sequences for use in recombinant yeast host cells include but are not limited to the PGK1 and TPS1 termination sequences.

The recombinant polynucleotide can further include additional elements. Non-limiting examples of such additional elements include enhancer sequences, response elements, protein recognition sites, inducible elements, protein-binding sequences, 5′ and 3′ untranslated regions, polyadenylation sequences, introns, origins of replication, operators (i.e., polynucleotide adjacent to a promoter that comprise a protein-binding domain where a repressor protein can bind and reduce or eliminate activity of the promoter), and selection markers (i.e., genes that encode proteins that can complement the host cell's auxotrophy, provide antibiotic resistance, or result in a color change). Such elements are known in the art. Non-limiting examples of origins of replication include AMA1 and ANSI. Non-limiting examples of suitable selection markers include amdS (acetamidase), argB (ornithine carbamoyltransferase), bar (phosphinothricin acetyltransferase), hph (hygromycin phosphotransferase), niaD (nitrate reductase), pyrG (orotidine 5′-phosphate decarboxylase), sC (sulfate adenyltransferase), and trpC (anthranilate synthase), and derivatives thereof. In some embodiments, the selection marker comprises an alteration that decreases production of the selective marker, thus increasing the number of copies needed to permit a host cell comprising the polynucleotide to survive under selection.

In embodiments in which the recombinant polynucleotide comprises two or more expression cassettes, the operably linked promoters, optional signal sequences, sequences encoding polypeptide termination sequences, and optional additional elements can be identical or different between the two or more expression cassettes.

### Recombinant Host Cell

Methods for introducing a recombinant polynucleotide into a host cell to obtain a recombinant host cell are well-known in the art. Non-limiting examples of such methods include calcium phosphate transfection, dendrimer transfection, liposome transfection (e.g., cationic liposome transfection), cationic polymer transfection, cell squeezing, sonoporation, optical transfection, protoplast fusion, impalefection, hyrodynamic delivery, gene gun, magnetofection, viral transduction, electroporation and chemical transformation (e.g., using PEG).

The recombinant polynucleotide can be maintained extra-chromosomal in the recombinant host cell on an expression vector (i.e., a polynucleotide that transduces, transforms, or infects a host cell, and causes it to express a polynucleotide and/or a polypeptide other than those native to the host cell, or in a manner not native to the host cell), or can be stably integrated within the genome (e.g., a chromosome) of the recombinant host cell. For integration into the genome, the recombinant polynucleotide can comprise sequences for integration into the genome by homologous (i.e., targeted integration) or nonhomologous recombination. The recombinant polynucleotide may comprise at least 100, at least 250, at least 500, at least 750, at least 1,000, or at least 10,000 base pairs that are highly homologous with a target sequence in the genome of the recombinant host cell to enhance the probability of homologous recombination. Such highly homologous sequence may be non-coding or coding. More than one copy of the recombinant polynucleotide may be inserted into the recombinant host cell to increase production of the recombinant milk protein.

The recombinant host cell can be obtained from host cells obtained from any organism, including but not limited to any animal, plant, algae fungus (e.g., yeast, filamentous fungus), or bacterium, as well as any generally recognized as safe (GRAS) industrial strain.

Non-limiting examples of suitable plants include cycad, Ginkgo biloba, conifer, cypress, juniper, thuja, cedarwood, pine, angelica, caraway, coriander, cumin, fennel, parsley, dill, dandelion, helichrysum, marigold, mugwort, safflower, camomile, lettuce, wormwood, calendula, citronella, sage, thyme, chia seed, mustard, olive, coffee, capsicum, eggplant, paprika, cranberry, kiwi, vegetables (e.g., carrot, celery), tagete, tansy, tarragon, sunflower, wintergreen, basil, hyssop, lavender, lemon verbena, marjoram, melissa, patchouli, pennyroyal, peppermint, rosemary, sesame, spearmint, primrose, samara, pepper, pimento, potato, sweet potato, tomato, blueberry, nightshade, petunia, morning glory, lilac, jasmin, honeysuckle, snapdragon, psyllium, wormseed, buckwheat, amaranth, chard, quinoa, spinach, rhubarb, jojoba, cypselea, chlorella, manila, hazelnut, canola, kale, bok Choy, rutabaga, frankincense, myrrh, elemi, hemp, pumpkin, squash, curcurbit, manioc, dalbergia, legume plants (e.g., alfalfa, lentil, bean, clover, pea, fava coceira, frijole bola roja, frijole negro, lespedeza, licorice, lupin, mesquite, carob, soybean, peanut, tamarind, wisteria, cassia, chickpea/garbanzo, fenugreek, green pea, yellow pea, snow pea, lima bean, fava bean), geranium, flax, pomegranate, cotton, okra, neem, fig, mulberry, clove, eucalyptus, tea tree, niaouli, fruiting plant (e.g., apple, apricot, peach, plum, pear, nectarine), strawberry, blackberry, raspberry, cherry, prune, rose, tangerine, citrus (e.g., grapefruit, lemon, lime, orange, bitter orange, mandarin, tangerine), mango, citrus bergamot, buchu, grape, broccoli, brussels sprout, camelina, cauliflower, rape, rapeseed (canola), turnip, cabbage, cucumber, watermelon, honeydew melon, zucchini, birch, walnut, cassava, baobab, allspice, almond, breadfruit, sandalwood, macadamia, taro, tuberose, aloe vera, garlic, onion, shallot, vanilla, yucca, vetiver, galangal, barley, corn, curcuma aromatica, ginger, lemon grass, oat, palm, pineapple, rice, rye, sorghum, triticale, turmeric, yam, bamboo, barley, cajuput, canna, cardamom, maize, oat, wheat, cinnamon, sassafras, lindera benzoin, bay laurel, avocado, ylang-ylang, mace, nutmeg, moringa, horsetail, oregano, cilantro, chervil, chive, aggregate fruit, grain plant, herbal plant, leafy vegetable, non-grain legume plant, nut plant, succulent plant, land plant, water plant, delbergia, millet, drupe, schizocarp, flowering plant, non-flowering plant, cultured plant, wild plant, tree, shrub, flower, grass, herbaceous plant, brush, lianas, cacti, tropical plant, subtropical plant, temperate plant, and moss (e.g., Physcomitrella patens).

Non-limiting examples of suitable algea include members of any of the following genera, and derivatives or crosses thereof: red algae, brown algae, gree algae, microalgae, Acinetobacter, Achnanthes (e.g., Achnanthes orientalis), Agmenellum, Alaria (e.g., Alaria marginata), Amphiprora (e.g., Amphiprora hyaline), Amphora (e.g., Amphora coffeiformis, Amphora coffeiformis linea, Amphora coffeiformis punctata, Amphora coffeiformis taylori, Amphora coffeiformis tenuis, Amphora delicatissima, Amphora delicatissima capitata, Amphora sp.), Anabaena, Analipus (e.g., Analipus japonicus), Ankistrodesmus (e.g., Ankistrodesmus falcatus), Ascophyllum (e.g., Ascophyllum nodosum), Boekelovia (e.g., Boekelovia hooglandii), Borodinella (e.g., Borodinella sp.), Botryococcus (e.g., Botryococcus braunii, Botryococcus sudeticus), Carteria, Chaetoceros (e.g., Chaetoceros gracilis, Chaetoceros muelleri, Chaetoceros muelleri subsalsum, Chaetoceros sp.), Chlorella (e.g., Chlorella anitrata, Chlorella Antarctica, Chlorella aureoviridis, Chlorella candida, Chlorella capsulate, Chlorella desiccate, Chlorella ellipsoidea, Chlorella emersonii, Chlorella fusca, Chlorella fusca var. vacuolata, Chlorella glucotropha, Chlorella infusionum, Chlorella infusionum var. actophila, Chlorella infusionum var. auxenophila, Chlorella kessleri, Chlorella lobophora (strain SAG 37.88), Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella parva, Chlorella photophila, Chlorella pringsheimii, Chlorella protothecoides, Chlorella protothecoides var. acidicola, Chlorealla, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris, Chlorella vulgaris f tertia, Chlorella vulgaris var. autotrophica, Chlorella vulgaris var. viridis, Chlorella vulgaris var. vulgaris, Chlorella vulgaris var. vulgaris f tertia, Chlorella vulgaris var. vulgaris f viridis, Chlorella xanthella, Chlorella zofingiensis, Chlorella trebouxioides, Chlorella vulgaris), Chlorococcum (e.g., Chlorococcum infusionum, Chlorococcum sp.), Chlorogonium, Chondrus (e.g., Chondrus crispus, Chondrus ocellatus), Chroomonas (e.g., Chroomonas sp.), Chrysosphaera (e.g., Chrysosphaera sp.), Cricosphaera (e.g., Cricosphaera sp.), Cryptomonas (e.g., Cryptomonas sp.), Cyclotella (e.g., Cyclotella cryptica, Cyclotella meneghiniana, Cyclotella sp.), Dunaliella (e.g., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta), Ecklonia (e.g., Ecklonia sp), Eisenia (e.g., Eisenia bicyclis), Ellipsoidon (e.g., Ellipsoidon sp.), Eremosphaera (e.g., Eremosphaera viridis, Eremosphaera sp.), Eucheuma (e.g., Eucheuma cottonii, Eucheuma spinosum), Euglena, Fragilaria (e.g., Fragilaria crotonensis, Fragilaria sp.), Franceia (e.g., Franceia sp.), Furcellaria (e.g., Furcellaria fastigiate), Gigartina (e.g., Gigartina acicularis, Gigartina bursa-pastoris, Gigartina pistillata, Gigartina radula, Gigartina skottsbergii, Gigartina stellate), Gleocapsa (e.g., Gleocapsa sp.), Gloeothamnion (e.g., Gloeothamnion sp.), Gloiopeltis (e.g., Gloiopeltis furcate), Gracilaria (e.g., Gracilaria bursa-pastoris, Gracilaria lichenoides), Hizikia (e.g., Hizikia fusiforme), Hymenomonas (e.g., Hymenomonas sp.), Isochrysis (e.g., Isochrysis aff. galbana, Isochrysis galbana), Kjellmaniella (e.g., Kjellmaniella gyrate), Laminaria (e.g., Laminaria angustata, Laminaria longirruris, Laminaria Longissima, Laminaria ochotensis, Laminaria claustonia, Laminaria saccharina, Laminaria digitata, Laminaria japonica), Lepocinclis, Macrocystis (e.g., Macrocystis pyrifera), Micractinium, Monoraphidium (e.g., Monoraphidium minutum, Monoraphidium sp.), Nannochloris (e.g., Nannochloris sp.), Nannochloropsis (e.g., Nannochloropsis salina, Nannochloropsis sp.), Navicula (e.g., Navicula acceptata, Navicula biskanterae, Navicula pseudotenelloides, Navicula pelliculosa, Navicula saprophila, Navicula sp.), Nephrochloris (e.g., Nephrochloris sp.), Nephroselmis (e.g., Nephroselmis sp.), Nitzschia (e.g., Nitzschia communis, Nitzschia alexandrina, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp.), Ochromonas (e.g., Ochromonas sp.), Oocystis (e.g., Oocystis parva, Oocystis pusilla, Oocystis sp.), Oscillatoria (e.g., Oscillatoria limnetica, Oscillatoria sp., Oscillatoria subbrevis), Palmaria (e.g., Palmaria palmata), Pascheria (e.g., Pascheria acidophila), Pavlova (e.g., Pavlova sp.), Petalonia (e.g., Petalonia fascia), Phagus, Phormidium, Platymonas (e.g., Platymonas sp.), Pleurochrysis (e.g., Pleurochrysis carterae, Pleurochrysis dentate, Pleurochrysis sp.), Porphyra (e.g., Porphyra columbina, Porphyra crispata, Porhyra deutata, Porhyra perforata, Porhyra suborbiculata, Porphyra tenera), Porphyridium (e.g., Porphyridium cruentum, Porphyridium purpureum, Porphyridium aerugineum), Prototheca (e.g., Prototheca wickerhamii, Prototheca stagnora, Prototheca portoricensis, Prototheca moriformis, Prototheca zopfii), Pyramimonas (e.g., Pyramimonas sp.), Pyrobotrys, Rhodella (e.g., Rhodella maculate, Rhodella reticulata, Rhodella violacea), Rhodymenia (e.g., Rhodymenia palmata), Sarcinoid (e.g., Sarcinoid chrysophyte), Scenedesmus (e.g., Scenedesmus armatus), Scytosiphon (e.g., Scytosiphon lome), Spirogyra, Spirulina (e.g., Spirulina platensis), Stichococcus (e.g., Stichococcus sp.), Synechococcus (e.g., Synechococcus sp.), Tetraedron, Tetraselmis (e.g., Tetraselmis sp., Tetraselmis suecica), Thalassiosira (e.g., Thalassiosira weissflogii), and Viridiella (e.g., Viridiella fridericiana).

Non-limiting examples of suitable yeast include members of any of the following genera, and derivatives or crosses thereof: Candida (e.g., Candida albicans, Candida etchellsii, Candida guilliermondii, Candida humilis, Candida lipolytica, Candida orthopsilosis, Candida palmioleophila, Candida pseudotropicalis, Candida sp., Candida utilis, Candida versatilis), Cladosporium, Cryptococcus (e.g., Cryptococcus terricolus, Cryptococcus curvatus), Debaryomyces (e.g., Debaryomyces hansenii), Endomyces (e.g., Endomyces vernalis), Endomycopsis (e.g., Endomycopsis vernalis), Eremothecium (e.g., Eremothecium ashbyii), Hansenula (e.g., Hansenula sp., Hansenula polymorpha), Kluyveromyces (e.g., Kluyveromyces sp., Kluyveromyces lactis, Kluyveromyces marxianus var. lactis, Kluyveromyces marxianus, Kluyveromyces thermotolerans), Lipomyces (e.g., Lipomyces starkeyi, Lipomyecs lipofer), Ogataea (e.g., Ogataea minuta), Pichia (e.g., Pichia sp., Pichia pastoris, Pichia finlandica, Pichia trehalophila, Pichia koclamae, Pichia membranaefaciens, Pichia minuta, Pichia lindneri), Pichia opuntiae, Pichia thermotolerans, Pichia salictaria, Pichia guercuum, Pichia pijperi, Pichia stiptis, Pichia methanolica), Rhodosporidium (e.g., Rhodosporidium toruloides), Rhodotorula (e.g., Rhodotorula sp., Rhodotorula gracilis, Rhodotorula glutinis, Rhodotorula graminis), Saccharomyces (e.g., Saccharomyces sp., Saccharomyces bayanus, Saccharomyces beticus, Saccharomyces cerevisiae, Saccharomyces chevalieri, Saccharomyces diastaticus, Saccharomyces ellipsoideus, Saccharomyces exiguus, Saccharomyces florentinus, Saccharomyces fragilis, Saccharomyces pastorianus, Saccharomyces pombe, Saccharomyces sake, Saccharomyces uvarum), Sporobolomyces (e.g., Sporobolomyces roseus), Sporidiobolus (e.g., Sporidiobolus johnsonii, Sporidiobolus salmonicolor), Trichosporon (e.g., Trichosporon cacaoliposimilis, Trichosporon oleaginosus sp. nov., Trichosporon cacaoliposimilis sp. nov., Trichosporon gracile, Trichosporon dulcitum, Trichosporon jirovecii, Trichosporon insectorum), Xanthophyllomyces (e.g., Xanthophyllomyces dendrorhous), Yarrowia (e.g., Yarrowia lipolytica), and Zygosaccharomyces (e.g., Zygosaccharomyces rouxii).

Non-limiting examples of suitable filamentous fungi include holomorphic, teleomorphic, and anamorphic forms of fungi, including members of any of the following genera, and derivatives or crosss thereof: Acremonium (e.g., Acremonium alabamense), Aspergillus (e.g., Aspergillus aculeatus, Aspergillus awamori, Aspergillus clavatus, Aspergillus flavus, Aspergillus foetidus, Aspergillus fumigatus, Aspergillus japonicus, Aspergillus nidulans, Aspergillus niger, Aspergillus niger var. awamori, Aspergillus ochraceus, Aspergillus oryzae, Aspergillus sojae, Aspergillus terreus, as well as Emericella, Neosartorya, and Petromyces species), Aureobasidium, Canariomyces, Chaetomium, Chaetomidium, Corynascus, Chrysosporium (e.g., Chrysosporium botryoides, Chrysosporium carmichaeli, Chrysosporium crassitunicatum, Chrysosporium europae, Chrysosporium evolceannui, Chrysosporium farinicola, Chrysosporium fastidium, Chrysosporium filiforme, Chrysosporium georgiae, Chrysosporium globiferum, Chrysosporium globiferum var. articulatum, Chrysosporium globiferum var. niveum, Chrysosporium hirundo, Chrysosporium hi spanicum, Chrysosporium holmii, Chrysosporium indicum, Chrysosporium iops, Chrysosporium keratinophilum, Chrysosporium kreiselii, Chrysosporium kuzurovianum, Chrysosporium lignorum, Chrysosporium obatum, Chrysosporium lucknowense, Chrysosporium lucknowense Garg 27K, Chrysosporium medium, Chrysosporium medium var. spissescens, Chrysosporium mephiticum, Chrysosporium merdarium, Chrysosporium merdarium var. roseum, Chrysosporium minor, Chrysosporium pannicola, Chrysosporium parvum, Chrysosporium parvum var. crescens, Chrysosporium pilosum, Chrysosporium pseudomerdarium, Chrysosporium pyriformis, Chrysosporium queenslandicum, Chrysosporium sigleri, Chrysosporium sulfureum, Chrysosporium synchronum, Chrysosporium tropicum, Chrysosporium undulatum, Chrysosporium vallenarense, Chrysosporium vespertilium, Chrysosporium zonatum), Coonemeria, Cunninghamella (e.g., Cunninghamella ehinulata), Dactylomyces, Emericella, Filibasidium, Fusarium (e.g., Fusarium moniliforme, Fusarium venenatum, Fusarium oxysporum, Fusarium graminearum, Fusarium proliferatum, Fusarium verticiollioides, Fusarium culmorum, Fusarium crookwellense, Fusarium poae, Fusarium sporotrichioides, Fusarium sambuccinum, Fusarium torulosum, as well as associated Gibberella teleomorphic forms thereof), Gibberella, Humicola, Hypocrea, Lentinula, Malbranchea (e.g., Malbranchea filamentosa), Magnaporthe, Malbranchium, Melanocarpus, Mortierella (e.g., Mortierella alpina 1S-4, Mortieralla isabelline, Mortierrla vinacea, Mortieralla vinaceae var. raffinoseutilizer), Mucor (e.g., Mucor miehei Cooney et Emerson (Rhizomucor miehei (Cooney & R. Emerson)) Schipper, Mucor pusillus Lindt, Mucor circinelloides Mucor mucedo), Myceliophthora (e.g., Myceliophthora thermophila), Myrothecium, Neocallimastix, Neurospora (e.g., Neurospora crassa), Paecilomyces, Penicillium (e.g., Penicillium chrysogenum, Pennicillium iilacinum, Penicillium roquefortii), Phenerochaete, Phlebia, Piromyces, Pythium, Rhizopus (e.g., Rhizopus niveus), Schizophyllum, Scytalidium, Sporotrichum (e.g., Sporotrichum cellulophilum), Stereum, Talaromyces, Thermoascus, Thermomyces, Thielavia (e.g., Thielavia terrestris), Tolypocladium, and Trichoderma (e.g., Trichoderma harzianum, Trichoderma koningii, Trichoderma longibrachiatum, Trichoderma reesei, Trichoderma atroviride, Trichoderma virens, Trichoderma citrinoviride, Trichoderma viride).

Non-limiting examples of suitable bacteria include firmicute, cyanobacteria (blue-green algae), oscillatoriophcideae, bacillales, lactobacillales, oscillatoriales, bacillaceae, lactobacillaceae, and members of any of the following genera, and derivatives and crosses thereof: Acinetobacter, Acetobacter (e.g., Acetobacter suboxydans, Acetobacter xylinum), Actinoplane (e.g., Actinoplane missouriensis), Arthrospira (e.g., Arthrospira platensis, Arthrospira maxima), Bacillus (e.g., Bacillus cereus, Bacillus coagulans, Bacillus licheniformis, Bacillus stearothermophilus, Bacillus subtilis), Escherichia (e.g., Escherichia coli), Lactobacillus (e.g., Lactobacillus acidophilus, Lactobacillus bulgaricus), Lactococcus (e.g., Lactococcus lactis, Lactococcus lactis Lancefield Group N, Lactobacillus reuteri), Leuconostoc (e.g., Leuconostoc citrovorum, Leuconostoc dextranicum, Leuconostoc mesenteroides), Micrococcus (e.g., Micrococcus lysodeikticus), Rhodococcus (e.g., Rhodococcus opacus, Rhodococcus opacus strain PD630), Spirulina, Streptococcus (e.g., Streptococcus cremoris, Streptococcus lactis, Streptococcus lactis subspecies diacetylactis, Streptococcus thermophilus), Streptomyces (e.g., Streptomyces chattanoogensis, Streptomyces griseus, Streptomyces natalensis, Streptomyces olivaceus, Streptomyces olivochromogenes, Streptomyces rubiginosus), Tetrahymena (e.g., Tetrahymena thermophile, Tetrahymena hegewischi, Tetrahymena hyperangularis, Tetrahymena malaccensis, Tetrahymena pigmentosa, Tetrahymena pyriformis, Tetrahymena vorax), and Xanthomonas (e.g., Xanthomonas campestris).

The recombinant host cell can further comprise a genetic modification that improves production of the recombinant milk protein. Non-limiting examples of suitable genetic modifications include altered promoters, altered kinase activities, altered phosphatase activities, altered protein folding activities, altered protein secretion activities, and altered gene expression induction pathways.

The recombinant host cell can further have reduced or essentially eliminated activity of a protease so as to minimize degradation of the recombinant milk protein (see, for example, PCT application WO 96/29391). Recombinant host cells with reduced or essentially eliminated activity of a protease can be obtained by screening of mutants or by specific genetic modification as per methods known in the art.

The recombinant host cell can further comprise a native or recombinant glycosyltransferase. Non-limiting examples of suitable endogenous or recombinant glycosyltransferases include fucosyltransferases, galactosyltransferases, glucosyltransferases, xylosyltransferases, acetylases, glucoronyltransferases, glucoronylepimerases, sialyltransferases, mannosyltransferases, sulfotransferases, β-acetylgalactosaminyltransferases, and N-acetylglucosaminyltransferases.

The recombinant host cell can further comprise a native or recombinant kinase or phosphatase. Non-limiting examples of suitable native or recombinant kinases or phosphatases include protein kinase A, protein kinase B, protein kinase C, creatine kinase B, protein kinase C beta, protein kinase G, TmkA, Fam20 kinases (e.g., Fam20C), ATM, CaM-II, cdc2, cdk5, CK1, CKII, DNAPK, EGFR, GSK3, INSR, p38MAPK, RSK, SRC, phosphotransferases, alkaline phosphatase (e.g., UniProtKB—077578), acid phosphatase, and others (see, for example, Kabir & Kazi. 2011. Genet Mol Biol. 34(4):587).

### Culturing Recombinant Host Cell

Suitable conditions for production of the recombinant milk protein are those under which the recombinant host cell can grow and/or remain viable, and produce the recombinant milk protein provided herein. Non-limiting examples of suitable conditions include a suitable culture medium (e.g., a culture medium having a suitable nutrient content [e.g., a suitable carbon content, a suitable nitrogen content, a suitable phosphorus content], a suitable supplement content, a suitable trace metal content, a suitable pH), a suitable temperature, a suitable feed rate, a suitable pressure, a suitable level of oxygenation, a suitable fermentation duration (i.e., volume of culture media comprising the recombinant host cells), a suitable fermentation volume (i.e., volume of culture media comprising the recombinant host cells), and a suitable fermentation vessel.

Suitable culture media include all culture media in which the recombinant host cell can grow and/or remain viable, and produce the recombinant milk protein provided herein. Typically, the culture medium is an aqueous medium that comprises a carbon source, an assimilable nitrogen source (i.e., a nitrogen-containing compound capable of releasing nitrogen in a form suitable for metabolic utilization by the recombinant host cell), and a phosphate source.

Non-limiting examples of carbon sources include monosaccharides, disaccharides, polysaccharides, acetate, ethanol, methanol, glycerol, methane, and combinations thereof. Non-limiting examples of monosaccharides include dextrose (glucose), fructose, galactose, xylose, arabinose, and combinations thereof. Non-limiting examples of disaccharides include sucrose, lactose, maltose, trehalose, cellobiose, and combinations thereof. Non-limiting examples of polysaccharides include starch, glycogen, cellulose, amylose, hemicellulose, maltodextrin, and combinations thereof.

Non-limiting examples of assimilable nitrogen sources include anhydrous ammonia, ammonium sulfate, ammonium hydroxide, ammonium nitrate, diammonium phosphate, monoammonium phosphate, ammonium pyrophosphate, ammonium chloride, sodium nitrate, urea, peptone, protein hydrolysates, corn steep liquor, corn steep solids, spent grain, spent grain extract, and yeast extract. Use of ammonia gas is convenient for large scale operations, and can be employed by bubbling through the aqueous ferment (fermentation medium) in suitable amounts. At the same time, such ammonia can also be employed to assist in pH control.

The culture medium can further comprise an inorganic salt, a mineral (e.g., magnesium, calcium, potassium, sodium; e.g., in suitable soluble assimilable ionic and combined forms), a metal or transition metal (e.g., copper, manganese, molybdenum, zinc, iron, boron, iodine; e.g., in suitable soluble assimilable form), a vitamin, and any other nutrient or functional ingredient (e.g., a protease [e.g., a plant-based protease] that can prevent degradation of a recombinant milk protein, a protease inhibitor that can reduce the activity of a protease that can degrade the recombinant milk protein, and/or a sacrificial protein that can siphon away protease activity, an anti-foaming agent, an anti-microbial agent, a surfactant, an emulsifying oil).

Suitable culture media are available from commercial suppliers or may be prepared according to published compositions (e.g., in catalogues of the American Type Culture Collection).

A suitable pH can be a pH of between 2 and 8, 7.5, 7, 6.5, 6, 5.5, 5.4, 5.3, 5.2, 5.1, 5, 4.9, 4.8, 4.7, 4.6, 4.5, 4, 3.5, 3, or 2.5; between 2.5 and 8, 7.5, 7, 6.5, 6, 5.5, 5.4, 5.3, 5.2, 5.1, 5, 4.9, 4.8, 4.7, 4.6, 4.5, 4, 3.5, or 3; between 3 and 8, 7.5, 7, 6.5, 6, 5.5, 5.4, 5.3, 5.2, 5.1, 5, 4.9, 4.8, 4.7, 4.6, 4.5, 4, or 3.5; between 3.5 and 8, 7.5, 7, 6.5, 6, 5.5, 5.4, 5.3, 5.2, 5.1, 5, 4.9, 4.8, 4.7, 4.6, 4.5, or 4; between 4 and 8, 7.5, 7, 6.5, 6, 5.5, 5.4, 5.3, 5.2, 5.1, 5, 4.9, 4.8, 4.7, 4.6, or 4.5; between 4.5 and 8, 7.5, 7, 6.5, 6, 5.5, 5.4, 5.3, 5.2, 5.1, 5, 4.9, 4.8, 4.7, or 4.6; between 4.6 and 8, 7.5, 7, 6.5, 6, 5.5, 5.4, 5.3, 5.2, 5.1, 5, 4.9, 4.8, or 4.7; between 4.7 and 8, 7.5, 7, 6.5, 6, 5.5, 5.4, 5.3, 5.2, 5.1, 5, 4.9, or 4.8; between 4.8 and 8, 7.5, 7, 6.5, 6, 5.5, 5.4, 5.3, 5.2, 5.1, 5, or 4.9; between 4.9 and 8, 7.5, 7, 6.5, 6, 5.5, 5.4, 5.3, 5.2, 5.1, or 5; between 5 and 8, 7.5, 7, 6.5, 6, 5.5, 5.4, 5.3, 5.2, or 5.1; between 5.1 and 8, 7.5, 7, 6.5, 6, 5.5, 5.4, 5.3, or 5.2; between 5.2 and 8, 7.5, 7, 6.5, 6, 5.5, 5.4, or 5.3; between 5.3 and 8, 7.5, 7, 6.5, 6, 5.5, or 5.4; between 5.4 and 8, 7.5, 7, 6.5, 6, or 5.5; between 5.5 and 8, 7.5, 7, 6.5, or 6; between 6 and 8, 7.5, 7, or 6.5; between 6.5 and 8, 7.5, or 7; between 7 and 8, or 7.5; or between 7.5 and 8.

A suitable temperature can be a temperature of between 20° C. and 46° C., 44° C., 42° C., 40° C., 38° C., 36° C., 34° C., 32° C., 30° C., 28° C., 26° C., 24° C., or 22° C.; between 22° C. and 46° C., 44° C., 42° C., 40° C., 38° C., 36° C., 34° C., 32° C., 30° C., 28° C., 26° C., or 24° C.; between 24° C. and 46° C., 44° C., 42° C., 40° C., 38° C., 36° C., 34° C., 32° C., 30° C., 28° C., or 26° C.; between 26° C. and 46° C., 44° C., 42° C., 40° C., 38° C., 36° C., 34° C., 32° C., 30° C., or 28° C.; between 28° C. and 46° C., 44° C., 42° C., 40° C., 38° C., 36° C., 34° C., 32° C., or 30° C.; between 30° C. and 46° C., 44° C., 42° C., 40° C., 38° C., 36° C., 34° C., or 32° C.; between 32° C. and 46° C., 44° C., 42° C., 40° C., 38° C., 36° C., or 34° C.; between 36° C. and 46° C., 44° C., 42° C., 40° C., or 38° C.; between 38° C. and 46° C., 44° C., 42° C., or 40° C.; between 40° C. and 46° C., 44° C., or 42° C., between 42° C. and 46° C. or 44° C.; or between 44° C. and 46° C.

A suitable feed rate can be a feed rate of between 0.01 g and 0.2 g glucose equivalent per g dry cell weight (DCW) per hour.

A suitable pressure can be a pressure of between 0 psig and 50 psig, 40 psig, 30 psig, 20 psig, or 10 psig; between 10 psig and 50 psig, 40 psig, 30 psig, or 20 psig; between 20 psig and 50 psig, 40 psig, or 30 psig; between 30 psig and 50 psig, or 40 psig; or between 40 psig and 50 psig.

A suitable oxygenation can be an aeration rate of between 0.1 volumes of oxygen per liquid volume in the fermentor per minute (vvm) and 2.1 vvm, 1.9 vvm, 1.7 vvm, 1.5 vvm, 1.3 vvm, 1.1 vvm, 0.9 vvm, 0.7 vvm, 0.5 vvm, or 0.3 vvm; between 0.3 vvm and 2.1 vvm, 1.9 vvm, 1.7 vvm, 1.5 vvm, 1.3 vvm, 1.1 vvm, 0.9 vvm, 0.7 vvm, or 0.5 vvm; between 0.5 vvm and 2.1 vvm, 1.9 vvm, 1.7 vvm, 1.5 vvm, 1.3 vvm, 1.1 vvm, 0.9 vvm, or 0.7 vvm; between 0.7 vvm and 2.1 vvm, 1.9 vvm, 1.7 vvm, 1.5 vvm, 1.3 vvm, 1.1 vvm, or 0.9 vvm; between 0.9 vvm and 2.1 vvm, 1.9 vvm, 1.7 vvm, 1.5 vvm, 1.3 vvm, or 1.1 vvm; between 1.1 vvm and 2.1 vvm, 1.9 vvm, 1.7 vvm, 1.5 vvm, or 1.3 vvm; between 1.3 vvm and 2.1 vvm, 1.9 vvm, 1.7 vvm, or 1.5 vvm; between 1.5 vvm and 2.1 vvm, 1.9 vvm, or 1.7 vvm; between 1.7 vvm and 2.1 vvm or 1.9 vvm; or between 1.9 vvm and 2.1 vvm.

A suitable fermentation duration can be a fermentation duration of between 10 hours and 500 hours, 400 hours, 300 hours, 200 hours, 100 hours, 50 hours, 40 hours, 30 hours, or 20 hours; between 20 hours and 500 hours, 400 hours, 300 hours, 200 hours, 100 hours, 50 hours, 40 hours, or 30 hours; between 30 hours and 500 hours, 400 hours, 300 hours, 200 hours, 100 hours, 50 hours, or 40 hours; between 40 hours and 500 hours, 400 hours, 300 hours, 200 hours, 100 hours, or 50 hours; between 50 hours and 500 hours, 400 hours, 300 hours, 200 hours, or 100 hours; between 100 hours and 500 hours, 400 hours, 300 hours, or 200 hours; between 200 hours and 500 hours, 400 hours, or 300 hours; between 300 hours and 500 hours, or 400 hours; or between 400 hours and 500 hours.

A suitable fermentation volume can be between 1 L and 10,000,000 L, 5,000,000 L, 1,000,000 L, 500,000 L, 100,000 L, 50,000 L, 10,000 L, 5,000 L, 1,000 L, 500 L, 100 L, 50 L, or 10 L; between 10 L and 10,000,000 L, 5,000,000 L, 1,000,000 L, 500,000 L, 100,000 L, 50,000 L, 10,000 L, 5,000 L, 1,000 L, 500 L, 100 L, or 50 L; between 50 L and 10,000,000 L, 5,000,000 L, 1,000,000 L, 500,000 L, 100,000 L, 50,000 L, 10,000 L, 5,000 L, 1,000 L, 500 L, or 100 L; between 100 L and 10,000,000 L, 5,000,000 L, 1,000,000 L, 500,000 L, 100,000 L, 50,000 L, 10,000 L, 5,000 L, 1,000 L, or 500 L; between 500 L and 10,000,000 L, 5,000,000 L, 1,000,000 L, 500,000 L, 100,000 L, 50,000 L, 10,000 L, 5,000 L, or 1,000 L; between 1,000 L and 10,000,000 L, 5,000,000 L, 1,000,000 L, 500,000 L, 100,000 L, 50,000 L, 10,000 L, or 5,000 L; between 5,000 L and 10,000,000 L, 5,000,000 L, 1,000,000 L, 500,000 L, 100,000 L, 50,000 L, or 10,000 L; between 10,000 L and 10,000,000 L, 5,000,000 L, 1,000,000 L, 500,000 L, 100,000 L, or 50,000 L; between 50,000 L and 10,000,000 L, 5,000,000 L, 1,000,000 L, 500,000 L, or 100,000 L; between 100,000 L and 10,000,000 L, 5,000,000 L, 1,000,000 L, or 500,000 L; between 500,000 L and 10,000,000 L, 5,000,000 L, or 1,000,000 L; between 1,000,000 L and 10,000,000 L, or 5,000,000 L; or between 5,000,000 L and 10,000,000 L.

A suitable fermentation vessel can be any fermentation vessel known in the art. Non-limiting examples of fermentation vessels include culture plates, shake flasks, fermentors (e.g., stirred tank fermentors, airlift fermentors, bubble column fermentors, fixed bed bioreactors, laboratory fermentors, industrial fermentors, or any combination thereof), used at any suitable scale (e.g., small-scale, large-scale) and in any process (e.g., solid culture, submerged culture, batch, fed-batch, or continuous-flow).

### Purifying Native or Recombinant Milk Protein

Methods for purifying native or recombinant proteins (e.g., from milk or fermentation broths) to obtain preparations comprising native or recombinant proteins are well-known in the art (see, for example, Protein Purification, J C Janson and L Ryden, Eds., VCH Publishers, New York, 1989; Protein Purification Methods: A Practical Approach, ELV Harris and S Angel, Eds., IRL Press, Oxford, England, 1989), and can be adapted to purify native or recombinant milk proteins.

A milk protein can be purified on the basis of its molecular weight, for example, by size exclusion/exchange chromatography, ultrafiltration through membranes, gel permeation chromatography (e.g., preparative disc-gel electrophoresis), or density centrifugation.

A milk protein also can be purified on the basis of its surface charge or hydrophobicity/hydrophilicity, for example, by isoelectric precipitation, anion/cation exchange chromatography, isoelectric focusing (IEF), or reverse phase chromatography.

A milk protein also can be purified on the basis of its solubility, for example, by ammonium sulfate precipitation, isoelectric precipitation, surfactants, detergents, or solvent extraction.

A milk protein also can be purified on the basis of its affinity to another molecule, for example, by affinity chromatography, reactive dyes, or hydroxyapatite. Affinity chromatography can include the use of an antibody having a specific binding affinity for the milk protein, or nickel NTA for a His-tagged recombinant milk protein, or a lectin to bind to a sugar moiety on a recombinant milk protein, or any other molecule that specifically binds the milk protein. In some embodiments, the recombinant milk protein carries a tag that facilitates purification. Non-limiting examples of such tags include epitope tags and protein tags. Non-limiting examples of epitope tags include c-myc, hemagglutinin (HA), polyhistidine (6x-HIS), GLU-GLU, and DYKDDDDK (FLAG) epitope tags. Non-limiting examples of protein tags include glutathione-S-transferase (GST), green fluorescent protein (GFP), and maltose binding protein (MBP). An epitope or protein tag may be removed following isolation of the recombinant milk protein (e.g., via protease cleavage).

A recombinant milk protein that is secreted by a recombinant host cell can be purified directly from the culture medium. A recombinant milk protein that is not secreted by a recombinant host cell can be purified from a cell lysate.

A milk protein can be purified to a purity of greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, greater than 95%, greater than 97%, or greater than 99% relative to other components comprised in the fermentation broth or preparation, or to at least 2-fold, at least 3-fold, at least 4-fold, at least 5-fold, at least 6-fold, at least 7-fold, at least 8-fold, at least 9-fold, or at least 10-fold greater abundancy relative to other components comprised in the fermentation broth, or to a purity of greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, greater than 95%, greater than 97%, or greater than 99% by weight.

The identity of the milk protein can be confirmed and/or quantified by high performance liquid chromatography (HPLC), Western blot analysis, Eastern blot analysis, polyacrylamide gel electrophoresis, capillary electrophoresis, formation of an enzyme product, disappearance of an enzyme substrate, and 2-dimensional mass spectroscopy (2D-MS/MS) sequence identification.

Native or recombinant milk protein can further be post-processed. Post-processing may alter certain chemical and/or physical properties of the native or recombinant milk protein, including but not limited to size, charge, hydrophobicity, hydrophilicity, solvation, protein folding, and chemical reactivity.

Post-processing can comprise fragmenting (e.g., by chemical means or by exposure to protease enzymes [e.g., trypsin, pepsin]), heating (e.g., to remove protein aggregates), removing reactive sites (e.g., removing reactive sites of methionine and/or tryptophan residues by oxidation), modulating (e.g., via chemical, photochemical, and/or enzymatic strategies), cyclizing, biotinylating (i.e., attaching biotin), and conjugation to other elements (e.g., poly-ethylene-glycol, antibodies, liposomes, phospholipids, DNA, RNA, polynucleotides, sugars, disaccharides, polysaccharides, starches, cellulose, detergents, cell walls).

Post-processing can occur in a random manner or in a site-specific manner (e.g., at sulfhydryl groups of cysteine residues [e.g., for aminoethylation, formation of iodoacetamides, formation of maleimides, formation of Dha, covalent attachment via disulfide bonds, and desulfurization], at primary amine groups of lysine residues [e.g., for attachment of activated esters, sulfonyl chlorides, isothiocyanates, unsaturated aldehyde esters, and aldehydes], at phenolic hydroxyl groups of tyrosine residues, at specific allergenic epitopes [e.g., glycan groups]).

Native or recombinant milk protein can be spray dried or concentrated via evaporation (e.g., to obtain a powder).

One or more native and/or recombinant milk proteins can be combined to obtain a milk protein component, and optionally combined with one or more carbohydrates and/or one or more other ingredients to obtain an egg replacer provided herein. Heating can aid in hydrating powder compositions.

### Method for Producing Food Product

In another aspect, provided herein is a method for producing a food product provided herein.

A variety of recipes exist for preparing a food product, and any such recipe can be used in the method provided herein to produce a food product provided herein. The egg replacer according to any of the above can be used in such recipes in place of egg or in addition to egg. The ratio by mass of whole egg, egg white, and/or egg yolk replaced to egg replacer used can range from about 1:2 to about 50:1 (e.g., about 1:2, about 1:1.5, about 1:1, about 1.5:1, about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1, about 10:1, about 15:1, about 20:1, about 25:1, about 30:1, about 35:1, about 40:1, about 45:1, about 50:1). For production of the batters, doughs, baked/cooked food products, and pasta according to any of the above, the egg replacer is typically combined with other ingredients such as, for example, additional carbohydrates (e.g., sugar, starch, gum, flour, edible fiber), proteins (e.g., non-milk proteins, plant proteins, protein concentrates, protein isolates), lipids (e.g., any of the lipids disclosed herein), minerals (e.g., any of the minerals disclosed herein), pH and/or ionic strength adjusting agents, cream of tartar, baking soda, leavening agents, sweetening agents (e.g., any of the sweetening agents disclosed herein), fruit pieces, chocolate pieces, cocoa powder, hydrating agents (e.g., any of the hydrating agents disclosed herein), and mixtures thereof. For each 50 g of large whole egg (without shell) replaced, between 0 g and 50 g (e.g., about 50 g, about 45 g, about 40 g, about 35 g, about 30 g, about 25 g, about 20 g, about 15 g, about 10 g, or about 5 g) of a hydrating agent can, for example, be added to the recipe.

### EXAMPLES

The following examples are included to illustrate specific embodiments of the invention. The techniques disclosed in the examples represent techniques discovered by the inventors to function well in the practice of the invention; however, those of skill in the art should, in light of the present disclosure, appreciate that many changes can be made in the specific embodiments that are disclosed and still obtain a like or similar result without departing from the spirit and scope of the invention. Therefore, all matter set forth or shown in the examples is to be interpreted as illustrative and not in a limiting sense.

### Example 1: Production of Recombinant β-Lactoglobulin in Recombinant Trichoderma reesei Host Cell

A recombinant Trichoderma reesei host cell was generated by transforming Trichoderma reesei (Hypocrea jecorina) strain Qm6a with a polynucleotide comprising a Bos taurus β-lactoglobulin coding sequence. ELISA assays and SDS-PAGE gel analyses were performed with the supernatant samples to identify positive transformants.

The recombinant Trichoderma reesei host cell was grown in a minimal basal media containing inorganic salts as sources of phosphate, ammonium, magnesium, potassium, sodium, sulfate, chloride, calcium, iron, manganese, zinc, molybdenum, copper, cobalt, and borate, with a carbohydrate-based carbon source as a starting feedstock, in a stirred fermentation vessel controlled at temperatures ranging between 25° C. through 34C, at aeration rates between 0.2 vvm and 1 vvm, and a minimum agitation to ensure proper mixing and dispersion of biomass and nutrients including oxygen (as delivered in compressed air). The pH of the fermentation broth was controlled at various setpoints ranging from 3.0 to 5.5 with on-demand addition of ammonium hydroxide. Once the batch carbohydrate was depleted, a solution containing glucose or lactose was added via a programmed feed recipe that delivered the carbon source at specific feed rates ranging from 0.01 through 0.1 g dry substrate per gram DCM per hour. The oxygen demand of the culture was satisfied by controlling agitation rate as demanded to maintain a target dissolved oxygen set point ranging from 5% to 50%. When agitation was no longer able to maintain the target dissolved oxygen set-point, aeration was increased up to 2.0 vvm. Once the culture was running at maximum agitation and aeration, the dissolved oxygen was allowed to drop below setpoint with no further actions taken. Various antifoams, including but not limited to ACP 1500, Antifoam 204 (Sigma), Erol DF6000K, Hodag K-60K, Industrol DF204 (BASF), P-2000E (Dow), SAG 471, SAG 5693, SAG 710, SAG 730, Silicone Antifoam (Sigma), Struktol J647, Struktol J673A, and sunflower oil were added as needed to control foaming in the fermentations. Each fermentation was harvested after at least 120 h, at biomass concentrations of typically between 20 g and 50 g DCW per L. Biomass was removed from the broth by centrifugation at 5,000×g, and the recombinant β-lactoglobulin was purified.

### Example 2: Production and Foaming of Egg Replacer

A recombinant β-lactoglobulin of Example 1 was used to produce an egg replacer comprising 10% by mass of a milk protein component consisting of the recombinant β-lactoglobulin, 12.8% by mass of sucrose, and 76.6% by mass of RO water, and pH was adjusted to 7.0 with NaOH.

The mass of the egg replacer needed to fill 3 separate 4 oz cups was determined (i.e., mass of solution).

The egg replacer was then whipped in a KitchenAid Artisan 5.0 L stand mixer for 20 min at speed setting 8 (planetary rpm of 225, beater rpm of 737) at 20° C. to obtain a foam.

Foaming capacity was determined by distributing the foam to 3 separate 4 oz cups, determining the mass of foam in each, and calculating overrun and air phase volume. Overrun was calculated as follows: overrun %=(mass of unwhipped solution−mass of foam)/(mass of foam)×100. Air phase volume was calculated as follows: air phase volume=overrun %/(overrun %+100). Results obtained with the 3 separate samples were averaged.

Foam strength was determined within 10 minutes of foam generation by placing about 50 mL of the foam into each of 3 aluminum sample cups and running a shear stress sweep in a MCR 102 rheometer (Anton Paar USA, Ashland, Va.). The inflection point where a sample exhibited strain was determined to be the yield stress. Results obtained with the 3 separate samples were averaged.

Foam stability was determined by punching a 5 mm hole in the bottom of a standard weigh boat, filling the weight boat with foam, leveling off the foam to ensure consisting sample volume, weighing the boat to determine the mass of foam added, and finally recording the time needed for half of that weight to be lost (via weeping of the foam through the hole).

As shown in Table 1, egg replacers comprising a milk protein component that consisted of only native β-lactoglobulin or of only recombinant β-lactoglobulin had a foaming capacity, foam strength, and foam stability that was comparable or superior to that of dried egg white.

TABLE 1
Foaming capacities, foam stabilities, and foam strengths
of egg replacers comprising milk protein component
consisting of: native β-lactoglobulin (A), recombinant
β-lactoglobulin (B), and dried egg whites (C)

| | |
|---|---|
| Foaming Capacity | Foam Strength |

| | Overrun | Air Phase | Foam Stability | Yield |
|---|---|---|---|---|
| | (%) | Volume | t_{1/2} (min) | stress (Pa) |
| A | 1105 | 0.92 | 146 | 123 |
| B | 1028 | 0.91 | 140 | 122 |
| C | 623 | 0.86 | 101 | 107 |

Native β-lactoglobulin (A) = β-lactoglobulin extracted from whey protein isolate (BiPro, obtained from Agropur Dairy Cooperative, Longueuil, Canada).

Notably, the egg replacers comprising native β-lactoglobulin (A) or recombinant β-lactoglobulin (B) lacked the strong sulfur off-flavor that is characteristic of dried egg whites (C).

### Example 3: Production and Gelation of Egg Replacer in Cake

A recombinant β-lactoglobulin of Example 1 was used to produce a powdered egg replacer comprising 12.6% by mass of a milk protein component consisting of the recombinant β-lactoglobulin, 15.0% by mass of tapioca starch (Bob's Red Mill), and 2.0% by mass of xanthan gum (Grindsted Xanthan 80) (referred to in Example 3 as “milk protein component egg replacer”).

The same egg replacer mixture but lacking the recombinant β-lactoglobulin was produced as a control, comprising 15.0% by mass tapioca starch and 2.0% by mass xanthan gum (referred to in Example 3 as “milk protein component-free starch/gum mixture egg replacer”)

These egg replacers were tested in yellow cake, where foaming/leavening behavior and gelling/thickening/coagulating behavior of ingredients (e.g., egg in conventional yellow cake, and egg replacer in yellow cake provided herein) are essential for rise/height, and an integral contributor to other attributes (e.g., physical, mechanical, chemical/biological, and sensory attributes).

Three recipe variants were tested. Each batch of cake batter was made using 1 box Betty Crocker Super Moist Yellow Cake Mix (General Mills, Minneapolis, Minn.), 105 g canola oil, 240 g RO water, and: 150 g egg, or 150 g of the milk protein component egg replacer or 150 g of the milk protein component-free starch/gum mixture egg replacer. Powdered forms of the milk protein component egg replacer and the milk protein component-free starch/gum mixture egg replacer were each first mixed with the boxed cake mix powder, and then hydrated with the RO water and canola oil (the mass of RO water being equal to the sum of the cake recipe RO water (i.e., 240 g) and the mass of RO water needed to hydrate the milk protein component egg replacer and the milk protein component-free starch/gum mixture egg replacer to their 150 g hydrated forms). One batch of cake batter makes two cakes.

Cake batter was mixed in a KitchenAid Professional 6000 HD 6.0 qt stand mixer with paddle attachment for 30 s at speed setting 2 (planetary rpm of 51, paddle rpm of 132), then residual dry cake mix clumps were scraped off the mixing bowl and paddle, followed by an additional 30 s at speed setting 5 (planetary rpm of 110, paddle rpm of 285).

Two 8 inch round aluminum cake pans with removable bottoms (#PCC-83, Fat Daddio's, Spokane, Wash.) were sprayed with a non-stick cooking spray (PAM Original, Conagra, Chicago, Ill.), and 450 g of cake batter was poured into each pan. The pans were then loaded into a 350° F. oven and baked until done (27-28 min total, rotating each pan after the first 20 min; doneness verified with a cake tester inserted at cake center). Cakes were set on cooling racks to cool, and de-panned after a minimum of 15 min (cakes remained on removable pan bottoms). Cakes were cooled at room temperature for a minimum of 1 hr before further analysis.

Some cakes were then cut in half for rise/height measurements to be taken, and other cakes were instead analyzed for mechanical attributes using a texture analyzer machine. After height measurements or texture analyzer testing, cakes were subjectively evaluated for color (i.e., browning) and sensory attributes.

To measure rise/height, cake rounds were cut in half along a diameter giving two semicircles of cake with a flat vertical cut surface; and one semicircle from each cake was used for measurements. The height at center of each cake was measured at the horizontal midpoint of the vertical cut surface; the height at ½ radius of each cake was measured at two locations—at 2 inches right and 2 inches left of the midpoint—and then averaged; and the height at side was measured at two locations—the leftmost and rightmost “shoulders” of the cake (i.e., the points at which the sides of the cake meet the top of the cake)—and then averaged. Height data was collected from 4 cakes of each recipe variant and averaged. To measure dome shape, two calculations were then performed using the averaged height data: (Height at ½ radius)−(Height at side) [a proxy for the slope of the dome for the outer portion of the cake (i.e., a ring bounded by the cake ½ radius and the cake circumference)] and (Height at center)−(Height at ½ radius) [a proxy for the slope of the dome for the inner portion of the cake (i.e., from the cake ½ radius to the center point)]. For a cake with a convex dome shape, the outer portion of the cake will have a steeper slope (i.e., a larger height difference value) than the inner portion of the cake, because the dome naturally flattens toward the center point (if it did not flatten, and the slope remained the same for both portions of the cake, the dome shape would be that of a (pointed) cone). A larger height difference value indicates a steeper slope (Table 2, below).

Mechanical properties of the cakes were measured with a TA.XT plus C Texture Analyzer (Stable Micro Systems Ltd, Surrey, England) with a conical probe (right circular cone with 22 mm height, 27 mm base diameter, 60° apex angle (i.e., angle of the cone tip), acrylic) performing a double compression test, to a depth of 11.5 mm. Immediately before testing a cake on the texture analyzer, the top of that cake was sliced off with a cake leveler at 25 mm of height (including 1 mm removable pan bottom, i.e., at 24 mm cake height), exposing a flat cross-section of the cake's interior crumb. Five double compression tests were then performed: one at the center of the cake round, and four distributed around the center at approximately midway between the cake center and the cake perimeter (with test locations adjusted slightly to avoid obvious large bubbles if present). A minimum of two cakes of each recipe variant were evaluated in this way, giving a minimum of 10 double compression tests per recipe variant.

During the double compression test, the texture analyzer probe twice descends and ascends into the cake sample through the 11.5 mm test depth at a slow constant rate (0.83 mm/sec, with a momentary pause (5.0 sec) between the completion of 1st ascent and the beginning of 2nd descent; i.e., START-DOWNSTROKE-UPSTROKE-MOMENTARY-PAUSE-DOWNSTROKE-UPSTROKE-END), and the instrument's load cell detects the instantaneous force exerted on the probe as it moves at that constant rate (with a trigger force of 0.049 N). The resulting data from a single double compression test is best visualized as a two-peaked curve on a plot with time as the x-axis and force as the y-axis. For the cakes tested, the shape of the curve was generally: START (0 force)-DOWNSTROKE into sample (from 0, force increases as probe descends further, up to a peak force experienced at 11.5 mm depth (maximum cake compression))-UPSTROKE from compressed sample (from peak force, force now decreases as probe ascends further, reaching 0 force and continuing into the negative, then rising back up to 0 force when probe has separated from and lifted above the sample (negative force region due to adhesion of sample to probe))-MOMENTARY-PAUSE (sample recovers, springing back (0 force))-DOWNSTROKE into sample (from 0, force increases as probe descends further, up to a peak force)-UPSTROKE from compressed sample (from peak force, force decreases as probe ascends further, reaching 0 force and continuing into the negative, then rising back up to 0 force when probe has separated from and lifted above the sample)-END (0 force).

The peak force recorded from the first compression is always larger than the peak force recorded from the second compression because the cake's structure is damaged during the first compression, so the cake's structure resists the probe's second compression with less force. The area under the curve for the second compression (i.e., work during second compression) is also less than the area under the curve for the first compression (i.e., work during first compression) due to cake structural damage. Because the damaged cake sample does not rebound fully to its original height after the first compression, the 2nd downstroke registers a force of 0 as it descends past 25 mm until it makes contact with the sample surface at a new lower height, at a later time. Area under the curve for a given compression can be subdivided into a downstroke portion (i.e., downstroke energy of the compression) and an upstroke portion (i.e., upstroke energy of the compression). During the downstroke, a portion of the downstroke energy is stored in the sample as elastic potential energy, and during the upstroke, a portion of that elastic potential energy is exerted by the sample upon the ascending probe as the upstroke energy. The upstroke energy of a compression is thus always less than the preceding downstroke energy of compression (though an ideal, perfectly elastic sample would have an equivalent downstroke energy and upstroke energy). During the upstroke, when the probe ascends beyond the compressed-and-rebounding sample, the force registered goes into the negative as the probe pulls away from the sample surface (which has adhered to the probe).

A number of output parameters can be calculated from a single double compression test curve, with different parameters giving insights into different properties of the cake sample. Exponent Connect software (version 7.0.3.0, Stable Micro Systems Ltd, Surrey, England) was used to calculate output parameters for each double compression test individually; and these parameters, averaged by recipe variant, are presented in Table 3 (below). Peak Force (i.e., hardness/firmness) is the maximum force during the first compression, conveying information on the hardness/firmness of the sample; a higher Peak Force means the sample resisted the downstroke with more force, and is likely a firmer sample. The First Upstroke Negative Work (i.e., adhesiveness) conveys information on the sample's adhesiveness, as the upstroke's negative force portion of the curve reflects the sample's adherence to the probe; a more-negative value implies a more-adhesive sample. Recoverable Energy (i.e., resilience) is calculated by dividing the first upstroke energy by the first downstroke energy, and conveys information on the sample's ability to store and expend downstroke energy in an elastic fashion; a value of 100% represents an ideal, perfectly elastic sample. Ratio of Work-Second to First Compression (i.e., structure integrity/cohesiveness) is calculated by dividing the work during the second compression by the work during the first compression, and conveys information on the structural integrity/cohesiveness of the sample after having its structure damaged by the first compression; a higher value (i.e., closer to 1.0) implies a greater structural integrity after the first compression, whereas a lower value (i.e., closer to 0.0) implies a greater deal of structural damage from the first compression. Rebound (i.e., elasticity/springiness) is calculated by dividing the second downstroke sample contact time (i.e., the length of time in which the probe registered a non-zero force during the second downstroke) by the first downstroke sample contact time; a value of 100% indicates that the sample rebounded fully to the initial height of contact from the first compression. The derived parameter of (Peak Force)×(Ratio of Work-Second to First Compression)×(Rebound) (i.e., chewiness/breakdown) conveys information on the general breakdown of a sample after the first compression, and a higher value may indicate what a human would consider a “chewier” sample and a lower value a “more tender” sample.

As shown in Table 2, cakes baked from batter comprising the milk protein component egg replacer (B) had a center height, ½ radius height, and side height that was comparable to that of cakes baked from batter comprising egg (A). In contrast, cakes baked from batter comprising the milk protein component-free starch/gum mixture egg replacer (C) had a center height, ½ radius height, and side height that was not comparable to that of cakes baked from batter comprising egg (A). As conveyed by the (Height at ½ radius)−(Height at side) and (Height at center)−(Height at ½ radius) difference values, cakes baked from batter comprising the milk protein component egg replacer (B) had a (very convex) dome shape that was comparable to that of cakes baked from batter comprising egg (A); while in contrast, cakes baked from batter comprising the milk protein component-free starch/gum mixture egg replacer (C) had a (less convex) dome shape that was not comparable to that of cakes baked from batter comprising egg (A).

TABLE 2
Height comparison of cakes baked from batter using: egg
(A), milk protein component egg replacer (B), and milk
protein component-free starch/gum mixture egg replacer (C)

| | | Height | | | (Height at |
|---|---|---|---|---|---|
| | Height | at 1/2 | Height at | (Height at 1/2 | center) - (Height |
| | at side | radius | center | radius) - (Height | at 1/2 radius) |
| | (mm) | (mm) | (mm) | at side) (mm) | (mm) |
| A | [19, 22] | [44, 46] | [50, 51] | 24 | 6 |
| B | [18, 20] | [42, 43] | [47, 49] | 23 | 6 |
| C | [17, 19] | [33, 35] | [37, 41] | 16 | 5 |

Hydrated milk protein component egg replacer (B) = 12.6% by mass recombinant β-lactoglobulin, 15.0% by mass tapioca starch, 2.0% by mass xanthan gum, and RO water for remainder.
Hydrated milk protein component-free starch/gum mixture egg replacer = 15.0% by mass tapioca starch, 2.0% by mass xanthan gum, and RO water for remainder.
Intervals represent the mean +/− the standard deviation.

As shown in Table 3, cakes baked from batter using a milk protein component egg replacer (B) had compression parameter values for Peak Force, First Upstroke Negative Work, Recoverable Energy, Ratio of Work-Second to First Compression, Rebound, and (Peak Force)×(Ratio of Work-Second to First Compression)×(Rebound) that were comparable to those of cakes baked from batter comprising egg (A). In contrast, cakes baked from batter using the milk protein component-free starch/gum mixture egg replacer (C) had compression parameter values that were not comparable to that of cakes baked from batter comprising egg (A).

TABLE 3
Texture analyzer parameters comparing cakes baked from batter
using: egg (A), milk protein component egg replacer (B), and
milk protein component-free starch/gum mixture egg replacer (C)

| | | | | | | (Peak Force) × |
|---|---|---|---|---|---|---|
| | | | | | | (Ratio of |
| | | | | Ratio of | | Work-Second |
| | | First Upstroke | | Work-Second | | to First |
| | Peak Force | Negative Work | Recoverable | to First | | Compression) × |
| | (N) | (N.sec) | Energy (%) | Compression | Rebound (%) | (Rebound) |
| A | [0.71, 0.85] | [−0.075, −0.040] | [17.5, 20.1] | [0.50, 0.53] | [79.6, 121.9] | [30.2, 52.5] |
| B | [0.84, 1.01] | [−0.041, −0.022] | [19.9, 22.8] | [0.51, 0.54] | [70.5, 127.3] | [33.2, 65.6] |
| C | [0.52, 0.59] | [−0.164, −0.134] | [12.3, 13.3] | [0.38, 0.40] | [47.0, 58.3] | [10.2, 13.3] |

Hydrated milk protein component egg replacer = 12.6% by mass recombinant β-lactoglobulin, 15.0% by mass tapioca starch, 2.0% by mass xanthan gum, and RO water for remainder.
Hydrated milk protein component-free starch/gum mixture egg replacer = 15.0% by mass tapioca starch, 2.0% by mass xanthan gum, and RO water for remainder.
Intervals represent the mean +/− the standard deviation.

Cakes from the three recipe variants were subjectively evaluated for color (i.e., browning) and eating quality. Cakes baked from batter using the milk protein component egg replacer (B) exhibited browning that was comparable to that of cakes baked from batter comprising egg (A). In contrast, cakes baked from batter using the milk protein component-free starch/gum mixture egg replacer (C) had a noticeably pale exterior that was not comparable to that of cakes baked from batter comprising egg (A). When cake samples were tasted/chewed/eaten, cakes baked from batter using the milk protein component egg replacer (B) were judged to have a pleasant mouthfeel and eating quality, with a balance of tenderness and springiness, and with the chew down feeling similar to that of cakes baked from batter comprising egg (A). In contrast, cakes baked from batter using the milk protein component-free starch/gum mixture egg replacer (C) had a very weak structure, were excessively tender and not sufficiently springy, and immediately turned to a sticky paste in the mouth upon chew down. Cakes baked from batter using the milk protein component egg replacer (B) had a sweet-eggy-vanilla baked aroma that was pleasing and had a flavor that was quite close (i.e. comparable) to that of cakes baked from batter comprising egg (A).

1-208. (canceled)

209. An egg replacer comprising a milk protein component consisting essentially of β-lactoglobulin, wherein the egg replacer comprises less than 20% by dry mass of protein obtained from plant, wherein the egg replacer is essentially free of an egg protein, and wherein the egg replacer is essentially free of a gluten.

210. The egg replacer of claim 209, wherein the egg replacer is essentially free of any protein other than the β-lactoglobulin comprised in the milk protein component.

211. The egg replacer of claim 209, wherein the β-lactoglobulin is a recombinant β-lactoglobulin.

212. The egg replacer of claim 211, wherein the recombinant β-lactoglobulin is at least 90% identical to bovine β-lactoglobulin.

213. The egg replacer of claim 211, wherein the recombinant β-lactoglobulin is at least 95% identical to bovine β-lactoglobulin.

214. The egg replacer of claim 211, wherein the recombinant β-lactoglobulin is at least 99% identical to bovine β-lactoglobulin.

215. The egg replacer of claim 211, wherein the recombinant β-lactoglobulin is produced in a recombinant host cell selected from the group consisting of a recombinant fungal host cell, a recombinant bacterial host cell, a recombinant algal host cell, or a recombinant plant host cell.

216. The egg replacer of claim 215, wherein the recombinant fungal host cell is a recombinant yeast host cell.

217. The egg replacer of claim 216, wherein the recombinant yeast host cell is derived from a member of a genus selected from the group consisting of Kluyveromyces, Pichia, Saccharomyces, and Yarrowia.

218. The egg replacer of claim 215, wherein the recombinant fungal host cell is a recombinant filamentous fungal host cell.

219. The egg replacer of claim 218, wherein the recombinant filamentous fungal host cell is derived from a member of a genus selected from the group consisting of: Aspergillus, Fusarium, Myceliophthora, and Trichoderma.

220. The egg replacer of claim 209, wherein the egg replacer comprises between about 5% and about 40% by mass of the milk protein component.

221. The egg replacer of claim 209, wherein the egg replacer comprises between about 10% and about 25% by mass of the milk protein component.

222. The egg replacer of claim 209, wherein the egg replacer further comprises a carbohydrate.

223. The egg replacer of claim 222, wherein the egg replacer comprises between about 10% and about 40% by mass of a starch.

224. The egg replacer of claim 223, wherein the starch is selected from the group consisting of tapioca starch, corn (maize) starch, potato starch, and any mixture of two or more thereof.

225. The egg replacer of claim 222, wherein the egg replacer comprises between about 1% and about 5% by mass of a gum.

226. The egg replacer of claim 223, wherein the gum is selected from the group consisting of xantham gum, tara gum, guar gum, agar gum, locust bean gum, or gum arabic, and any mixture of two or more thereof.

227. The egg replacer of claim 222, wherein the carbohydrate is a mixture of a starch and a gum.

228. The egg replacer of claim 227, wherein the starch is selected from the group consisting of tapioca starch, corn (maize) starch, potato starch, arrowroot starch, tapioca syrup solids, rice syrup solids, and any mixture of two or more thereof.

229. The egg replacer of claim 227, wherein the gum is selected from the group consisting of arrowroot flour, xanthan gum, tara gum, guar gum, agar gum, locust bean gum, gum arabic, and any mixture of two or more thereof.

230. The egg replacer of claim 227, wherein the starch is selected from the group consisting of tapioca starch, corn (maize) starch, potato starch, arrowroot starch, tapioca syrup solids, rice syrup solids, and any mixture of two or more thereof, and wherein the gum is selected from the group consisting of arrowroot flour, xanthan gum, tara gum, guar gum, agar gum, locust bean gum, gum arabic, and any mixture of two or more thereof.

231. The egg replacer of claim 227, wherein the egg replacer comprises between about 10% and about 40% by mass of the starch and between about 1% and about 5% by mass of the gum.

232. The egg replacer of claim 209, wherein the egg replacer is a powder.

233. A composition comprising the egg replacer of claim 209.

234. The composition of claim 233, wherein the composition is essentially free of an egg protein.

235. The composition of claim 233, wherein the composition is a food product.

236. The composition of claim 236, wherein the food product is selected from the group consisting of a batter, a dough, a baked/cooked food product, and an egg-based food product.

237. The composition of claim 236, wherein the composition is a baked food product; wherein the baked food product is essentially free of an egg protein, a gluten, or both; and wherein the baked food product has a similar cake rise/height as a comparable baked food product comprising an egg, protein, a gluten, or both.

238. A method for producing the egg replacer of claim 211, wherein the method comprises: obtaining a recombinant β-lactoglobulin.
